SEQUENCE LISTING (4 sequences)

SEQ ID NO: 1 - DNA, 16 bp, Artificial Sequence
  ASO 1316
acatacggctgtcttg

SEQ ID NO: 2 - DNA, 16 bp, Artificial Sequence
  ASO 1851
gatctaagaaggtgga

SEQ ID NO: 3 - DNA, 29 bp, Artificial Sequence
  Forward Primer
ccgagaggagtttccagacagacaggatc

SEQ ID NO: 4 - DNA, 25 bp, Artificial Sequence
  Reverse Primer
gctctgcacacttaacatctgctcg

(19) United States
(12) Patent Application Publication
(10) Pub. No.: US 2022/0265862 A1
(43) Pub. Date: Aug. 25, 2022

(54) COMPOSITIONS AND METHODS FOR THE TREATMENT OF LEUKODYSTROPHY AND WHOLE ANIMAL AND CELLULAR MODELS FOR IDENTIFYING EFFICACIOUS AGENTS FOR TREATMENT OF THE SAME

(71) Applicant: THE CHILDREN'S HOSPITAL OF PHILADELPHIA, Philadelphia, PA (US)

(72) Inventors: Akshata Almad, Philadelphia, PA (US); Adeline L. Vanderver, Bryn Mawr, PA (US)

(73) Assignee: THE CHILDREN'S HOSPITAL OF PHILADELPHIA, Philadelphia, PA (US)

(21) Appl. No.: 17/610,050

(22) PCT Filed: May 11, 2020
(86) PCT No.: PCT/US2020/032364

§ 371 Date: Nov. 9, 2021

Related U.S. Application Data
(60) Provisional application No. 62/924,910, filed on Oct. 23, 2019.
(60) Provisional application No. 62/845,637, filed on May 9, 2019.

(51) Int. Cl. A61K 48/00; A61K 49/00; A61K 31/7105; A61K 31/711; A61K 38/46; A61K 31/7088; A61K 38/17; A01K 67/027; C12N 5/0793; A61P 25/28; G01N 33/50; C12N 5/074; C12N 15/113

(52) U.S. Cl. CPC A61K 48/0066; C12N 2310/3231; A61K 31/7105; A61K 31/711; A61K 38/465; A61K 31/7088; A61K 38/1709; A01K 67/0278; C12N 5/0619; A61P 25/28; G01N 33/5088; C12N 5/0696; C12N 15/113; A01K 2217/056; A01K 2227/105; A01K 2267/0318; C12N 2506/45; C12N 2506/115; C12N 2310/14; C12N 2310/11; A61K 49/0008

(57) ABSTRACT

Compositions and methods for the treatment of leukodystrophy, particularly, H-ABC are disclosed.

### CROSS-REFERENCE TO RELATED APPLICATIONS

This application claims priority to U.S. Provisional Application No. 62/845,637 filed on May 9, 2019, and U.S. Provisional Application No. 62/924,910 filed Oct. 23, 2019, the entire disclosures of each of the aforementioned applications being incorporated herein by reference as though set forth in full.

### INCORPORATION-BY-REFERENCE OF MATERIAL SUBMITTED IN ELECTRONIC FORM

Incorporated herein by reference in its entirety is the Sequence Listing submitted via EFS-Web as a text file named SequenceListing.txt., created May 7, 2020 and having a size of 1,167 bytes.

### FIELD OF THE INVENTION

This invention relates to the fields of leukodystrophy and improved therapeutics for ameliorating symptoms of this disorder. The invention also provides a whole animal model for identifying agents useful for the treatment or prevention of leukodystrophy, particularly H-ABC.

### BACKGROUND OF THE INVENTION

Several publications and patent documents are cited throughout the specification in order to describe the state of the art to which this invention pertains. Each of these citations is incorporated herein by reference as though set forth in full.

Hypomyelination with Atrophy of Basal Ganglia and Cerebellum (H-ABC) is a leukodystrophy caused by sporadic, typically de novo heterozygous mutations in the TUBB4A gene (Simons et al., 2013a). This gene encodes the beta tubulin 4a protein, which heterodimerizes with alpha tubulin to assemble into microtubules. Monoallelic mutations in TUBB4A result in a spectrum of neurologic disorders ranging from an early onset leukoencephalopathy to adult-onset Dystonia type 4 (DYT4; whispering dysphonia). H-ABC affected individuals are within this spectrum, presenting in the toddler years, typically with dystonia (Hersheson et al., 2013), progressive gait impairment, speech and cognitive deficits. They are further delineated from other individuals with mutations in TUBB4A by characteristic neuroimaging features: hypomyelination and atrophy of the caudate and putamen along with cerebellar atrophy (van der Knaap et al., 2007).

On pathological specimens, dorsal striatal areas and the granular layer of the cerebellum exhibit neuronal loss with axonal swelling and diffuse paucity of myelin (Simons et al., 2013a; Curiel et al., 2017b). Individuals with characteristic features of H-ABC represent about 65% of published cases (Blumkin et al., 2014; Ferreira et al., 2014; Miyatake et al., 2014; Pizzino et al., 2014; Purnell et al., 2014) and are disproportionately likely to be affected by a single common mutation, p. Asp249Asn (24.1% of overall mutations in a cohort of 166 individuals, referred to hereafter as TUBB4A^{D249N}). H-ABC is currently considered an intermediary phenotype, between severely affected early infantile variants and juvenile-adult mild variants (Nahhas N, 2016).

TUBB4A is highly expressed in the central nervous system (CNS), particularly in the cerebellum and white matter tracts of the brain, with more moderate expression in the striatum (Hersheson et al., 2013). At a cellular level, TUBB4A is primarily localized to neurons and cells of the oligodendrocyte (OLs) lineage, with highest expression in mature myelinating OLs (Zhang et al., 2014). Although the expression pattern and associated disease phenotypes implicate functional role of beta tubulin 4a protein in both neuronal and oligodendrocyte populations, little is known about the pathologic mechanisms of TUBB4A mutations. Our group has reported the effect of a broader range of TUBB4A mutations using over-expression studies in an OL cell line as well as mouse cerebellar neurons. Over-expression of the Tubb4a^{D249N} mutation in an OL cell line resulted in decreased myelin gene expression and formation of immature OLs with fewer processes compared to Tubb4a^{WT} expression (Curiel et al., 2017b). Similarly, an abnormal neuronal phenotype with shorter axons, fewer dendrites and decreased dendritic branching was examined with the H-ABC causative Tubb4a^{D249N} mutation (Curiel et al., 2017b) compared to Tubb4a^{WT} expression. Other TUBB4A mutations highlighted phenotypic abnormalities specifically only in neurons and/or OL cell lines, suggesting mutation specific effects, corresponding to variable clinical phenotypes. A spontaneously occurring rat model, the taiep rat, with homozygous p. Ala302Thr Tubb4a mutation, has been reported with a hypomyelinating phenotype in the brain, optic nerves and certain tracts of the spinal cord. This specific mutation has not been previously seen in humans. An interesting feature observed in the taiep was accumulation of microtubules, particularly in the OLs along with subsequent demyelination (Duncan et al., 2017b). Currently there are no animal models for the p. Asp249Asn mutation specifically associated with H-ABC.

Clearly, a need exists for such models and for new therapeutic approaches for treating the debilitating disorders.

### SUMMARY OF THE INVENTION

In accordance with the present invention, a transgenic mouse model of H-ABC is provided. In one aspect, the mouse has a genome comprising: a promoter effective to drive expression in a mouse cell operably linked to a nucleic acid encoding a human, mutant Tubb4-a protein, wherein the human mutated Tubb4-protein is expressed in neurons and/or oligodendrocytes of the mouse and produces a leukodystrophy phenotype. Table 1 provides a list of nucleic acids encoding mutant Tubb4, which can be used in the animal models, cellular models, compositions and methods described herein. In certain embodiments, the mutated Tubb4-a protein is the Tubb4a^{D249N} variant.

The invention also provides a method for evaluating the presence of leukodystrophy in the transgenic mouse described above, comprising: measuring leukodystrophy symptoms selected from one or more of motor dysfunction, abnormal gait, ataxia, and decreased survival in the transgenic mouse, wherein presence of said symptoms relative to control mice which lack a variant TUBB4A transgene indicates that the mouse has leukodystrophy.

In another embodiment, a method of identifying a candidate compound for the treatment of leukodystrophy is provided. An exemplary method comprises contacting the transgenic mouse or a cell, tissue, or organ from neurons of the transgenic mouse with a test compound; and measuring levels of a physical parameter associated with leukodystropy in the animal, cell, tissue, or organ in the presence and absence of the test compound; and identifying a test compound that alters one or more of these parameters as a candidate compound. In certain embodiments, the physical parameter associated with leukodystrophy is selected from one or more of reduction in oligodendrocyte number, hypomyelination, cerebellar granular neuronal loss, striatal neuron loss, dysmyelination, myelination delay, abnormal gain, ataxia, and reduced neuronal survival.

In another aspect, a method of identifying a candidate therapeutic compound for the treatment of hypomyelination and atrophy of basal ganglia (H-ABC) is disclosed. An exemplary method comprises: exposing the transgenic mouse model of H-ABC to a test compound; measuring one or more parameters of leukodystrophy in the mouse in the presence and absence of the test compound; and identifying a test compound that improves the one or more parameters as a candidate therapeutic compound.

In yet another aspect, the invention provides a method of identifying a candidate therapeutic compound for the treatment of hypomyelination and atrophy of basal ganglia (H-ABC), comprising obtaining PBMCs from a subject harboring a TUBB-4A mutation and from control subjects which lack any TUBB4-A mutation; reprogramming monocytes from step a) to generate induced pluripotent stem cells (iPSCs); applying a dual SMAD inhibition protocol to direct differentiation of iPSCs towards a striatal spiny neuron fate, wherein the cells express one or more markers selected from DARPP32, CTIP2, GABA and FoxP1; and contacting said cells with said compound and assessing whether said compound alters a parameter associated with a leukodystrophy phenotype relative to control cells which lack said mutation. In some embodiments, the parameter is selected from one or more of reduced cell survival, altered spiny neuron marker expression, and altered cellular morphology or signaling. Cells can be obtained from patients harboring any of the TUBB-4A mutations is listed in Table 1.

Another approach for treating H-ABC entails administration of an effective amount of a compound which down modulates overall expression of TUBB4-A, thereby ameliorating symptoms of H-ABC. In certain aspects, the compound is selected from short hairpin RNA (shRNA), short interfering RNA (siRNA), antisense RNA, antisense DNA, chimeric Antisense DNA/RNA, microRNA, and ribozymes that are sufficiently complementary to either a gene or an mRNA encoding mutated TUBB4A.

In another approach for treatment, a method for genome editing of a TUBB4A encoding nucleic acid is disclosed. An exemplary method comprises administering to a subject a vector, wherein the subject is a human, and the vector comprises nucleic acid components of a CRISPR-mediated base editor 3 (BE3) system and a guide RNA (gRNA), the gRNA targeting a mutation in a TUBB4A gene. A modified codon is introduced in the therapeutic gene by base editing the therapeutic gene, wherein the base editing is performed by the vector.

Another method for the treatment or prevention of hypomyelination and atrophy of basal ganglia (H-ABC) leukodystrophy in a subject harboring a mutated TUBB4A gene, comprises administration of an effective amount of a compound which increases expression of wild type TUBB4-A protein, thereby ameliorating symptoms of H-ABC. In certain embodiments, increased expression is achieved by overexpression of wild-type TUBB4-A via introduction of an expression or viral vector, or nanoparticle comprising wild-type TUBB4-A encoding nucleic acids.

Finally, the invention provides kits for practicing the method of any of the foregoing claims.

### BRIEF DESCRIPTION OF THE DRAWINGS

FIGS. 1A-1M: Tubb4a^{D249N/D249N} mice show decreased survival, gait abnormalities and progressive motor dysfunction (FIG. 1A) Schematic diagram showing mouse Tubb4a gene and sequencing chart of WT, Tubb4a^{D249N} and Tubb4a^{D249N/D249N} mice. Red arrow demonstrates position 745 nucleotide in exon 4; WT shows one peak of G, Tubb4a^{D249N} mice show one peaks each of ‘G’ and “A’ and Tubb4a^{D249N/D249N} mice shows 2 peaks for T. (FIG. 1B) Representative image of the end-stage (ES) Tubb4a^{D249N/D249N} mouse (˜P35-P40) with severe dystonia and ataxia as compared to WT. (FIG. 1C) Kaplan-Meier survival curve of Tubb4a^{D249N/D249N} and Tubb4a^{D249N} mice as compared to WT littermates (Gehan-Breslow-Wilcoxon test, n=28). (FIG. 1D) Schematic diagram displaying the time course of behavioral tests. (FIG. 1E) Illustration of crawling and walking: For ambulation measurement, crawling and walking was scored (See Table 3); throughout crawling, the whole hind paw touches the ground as designated by (#) and tail is low or touching the ground (shown by red arrow). When transitioning from crawling to walking, head begins to rise. Walking is seen only when the toes of the hind paw touch the ground and the heel is elevated, designated by [##]. (FIG. 1F) Ambulatory deficits of Tubb4a^{D249N/D249N} mice at P7, P10 and P14. Statistical analysis by two-way ANOVA, followed by Tukey post-hoc analysis, n=10. (FIG. 1G) Representative images of ambulatory angles at P7, P21 and P35 of Tubb4a^{D249N} and Tubb4a^{D249N/D249N} as compared to WT littermates. (FIG. 1H) Measurement of ambulatory angles of Tubb4a^{D249N} and Tubb4a^{D249N/D249N} as compared to WT littermates at P7, P14, P21, P28 and P35. Statistical test by two-way ANOVA, followed by post-hoc Tukey test, n=14. (FIG. 11) Pictorial presentation of hanging grip strength set-up. (FIG. 1J) Grip strength as measured by inverted fall angle in Tubb4a^{D249N} and Tubb4a^{D249N/D249N} mice as compared to WTs Statistical test by one-way ANOVA, followed by Tukey post-hoc analysis, n=10. (FIG. 1K) Rota-rod testing demonstrating latency to fall (in seconds) in Tubb4a^{D249N} and Tubb4a^{D249N/D249N} mice at P21, P28 and P35 relative to WTs, n=14. (FIG. 1L) Graph for weight measurements of Tubb4a^{D249N} Tubb4a^{D249N/D249N} and WTs from P7, n=10. (FIG. 1M) Righting reflex changes of Tubb4a^{D249N} Tubb4a^{D249N/D249N} and WTs mice, n=14. Statistical tests performed by repeated measures two-way ANOVA, followed by Tukey post-hoc analysis. Data is presented as mean and SEM. *p<0.05, **p<0.01, ***p<0.001.

FIGS. 2A-2D Tubb4a^{D249N} mice show hypomyelination at 1 year (FIG. 2A) Kaplan-Meier survival curve of Tubb4a^{D249N} mice as compared to WTs (Gehan-Breslow-Wilcoxon test, n=10). (FIG. 2B) Rota-rod test performance at the age of 9 months and 1 year of Tubb4a^{D249N} mice relative to WTs, n=7-8. (FIG. 2C) Statistical test performed by one-way ANOVA, followed by Tukey post-hoc test. (FIG. 2D) PLP (green) in WT and Tubb4a^{D249N} mice at ES.

FIGS. 3A-3Z: Tubb4a^{D249N/D249N} mice exhibit severe developmental delay in myelination (FIG. 3A) Schematic diagram displaying the time course of immunohistochemical assays. (FIG. 3B) Schematic diagram showing analysis for corpus callosum (CC) and cerebellum (Cb). (FIGS. 3C-3D) Representative end-stage images (ES) (FIG. 3C) and quantification (FIG. 3D) of Eriochrome cyanine (Eri-C) stain (myelin [blue]) of WT, Tubb4a^{D249N} and Tubb4a^{D249N/D249N} in corpus callosum. Scale bar=250 μm (‘$$$’ indicates the significant difference between P21 and ES) (FIGS. 3E-3F) Representative ES images (FIG. 3E) and quantification (FIG. 3F) of Eri-C stain of WT, Tubb4a^{D249N} and Tubb4a^{D249N/D249N} in cerebellum. Scale bar 1 mm. (‘$$$’ indicates the significant difference between P21 and ES) (FIGS. 3G-3J) Representative images and quantification of loss of Eri-C staining (FIG. 3G and FIG. 3H) and MBP immunostaining (FIG. 3I and FIG. 3J) in CC seen in 1-year-old Tubb4a^{D249N/D249N} mice. (FIGS. 3K-3L) Representative end-stage images (FIG. 3K) and quantification (FIG. 3L) of PLP (green) at P14, P21 and ES of WT, Tubb4a^{D249N} and Tubb4a^{D249N/D249N} mice in corpus callosum. Scale bar=250 μm (FIGS. 3M-3N) Representative western blot images (FIG. 3M) and quantification (FIG. 3N) of normalized PLP protein levels at P21 and ES of WT, Tubb4a^{D249N} and Tubb4a^{D249N/D249N} mice in forebrain (FIGS. 3O-3P) Representative end-stage images (FIG. 3O) and quantification (FIG. 3P) of PLP (green) at P14, P21 and ES of WT, Tubb4a^{D249N} and Tubb4a^{D249N/D249N} mice in cerebellum. Scale bar=1 mm (FIGS. 3Q-3R) Representative western blot images (FIG. 3Q) and quantification (FIG. 3R) of normalized PLP protein levels at P21 and ES of WT, Tubb4a^{D249N} and Tubb4a^{D249N/D249N} mice in cerebellum. (FIGS. 3S-3T) Representative ES images (FIG. 3S) and quantification (FIG. 3T) of MBP (red) at P14, P21 and ES of WT, Tubb4a^{D249N} and Tubb4a^{D249N/D249N} mice in corpus callosum. Scale bar=250 μm (FIGS. 3U-3V) Representative western blot images (FIG. 3U) and quantification (FIG. 3V) of normalized MBP protein levels at P21 and ES of WT, Tubb4a^{D249N} and Tubb4a^{D249N/D249N} mice in forebrain. (FIGS. 3W-3X) Representative ES images (FIG. 3W) and quantification (FIG. 3X) of MBP (red) at P14, P21 and ES of WT, Tubb4a^{D249N} and Tubb4a^{D249N/D249N} mice in cerebellum. Scale bar=1 mm (FIGS. 3Y-3Z) Representative western blot images (FIG. 3Y) and quantification (FIG. 3Z) of normalized MBP protein levels at P21 and ES of WT, Tubb4a^{D249N} and Tubb4a^{D249N/D249N} mice in forebrain. Statistical test is performed by two-way ANOVA, followed by Tukey post-hoc test. Representative data with n=4 mice/group for P14 (except n=3 for Tubb4a^{D249N} for P14 time point) and P21 and n=3 mice/group for ES. Data is presented as mean and SEM. *p<0.05 and *** and $$$ p<0.001.

FIGS. 4A-4F: Electron Microscopy analysis of spinal cord shows that Tubb4a^{D249N} and Tubb4a^{D249/D249N} mice exhibit hypomyelination and dysmyelination (FIG. 4A-F) Representative electron microscopy (EM) images of ventral spinal cord from WT, Tubb4a^{D249N} and Tubb4a^{D249/D249N} mice at end-stage. Scale bar=1 μm (FIG. 4A-F) Higher magnification of spinal cord showing myelin sheath thickness in WT, Tubb4a^{D249N} and Tubb4a^{D249/D249N} animals with macrophage (M) mediated phagocytosis of axons (blue asterisks) in Tubb4a^{D249/D249N} tissue. Scale bar=800 nm

FIGS. 5A-5J: Electron Microscopy of optic nerve demonstrates that Tubb4a^{D249N} and Tubb4a^{D249/D249N} mice exhibit hypomyelination and dysmyelination (FIGS. 5A-5C and FIGS. 5H-5J) Representative electron microscopy (EM) images of optic nerve from WT, Tubb4a^{D249N} and Tubb4a^{D249/D249N} mice at end-stage. Scale bar=800 nm (FIGS. 5A-5C and FIGS. 5H-5J) Higher magnification of optic nerve showing myelin sheath thickness in WT, Tubb4a^{D249N} and Tubb4a^{D249/D249N} animals. Red asterisk=unmyelinated axons, black asterisks=thinly myelinated axons. Scale bar=400 nm (FIG. 5D) Macrophage (M) mediated phagocytosis of axons (blue asterisks) in Tubb4a^{D249/D249N} tissue. Scale bar=2 μm (FIG. 5E) G ratio measurements in optic nerve for WT, Tubb4a^{D249N} and Tubb4a^{D249/D249N} tissues (FIG. 5F) Scatter plot of G-ratio plotted against axon diameter for WT, Tubb4a^{D249N} and Tubb4a^{D249/D249N} tissues. (FIG. 5G) Axon diameter was plotted for all the groups. 50 axons per animals was counted with n=3 animals per group, data is presented as mean and SEM. One-way ANOVA was performed on the data set followed by Tukey post-hoc test. *p<0.05, ***p<0.001 FIGS. 6A-6G: Tubb4a^{D249/D249N} mice show hypomyelination at P21 (FIG. 6A) Schematic diagram displaying the time course of immunohistochemical assays. (FIG. 6B) Representative P21 images of Eriochrome cyanine (Eri-C) stain (myelin [blue]) of WT, Tubb4a^{D249N} and Tubb4a^{D249N/D249N} in corpus callosum. Scale bar=1 mm (FIG. 6C) Representative P21 images of PLP (green) of WT, Tubb4a^{D249N} and Tubb4a^{D249N/D249N} mice in corpus callosum. Scale bar=250 μm (FIG. 6D) Representative P21 images of MBP (red) of WT, Tubb4a^{D249N} and Tubb4a^{D249N/D249N} mice in corpus callosum. Scale bar=250 μm (FIG. 6E) Representative P21 images of Eriochrome cyanine (Eri-C) stain (myelin [blue]) of WT, Tubb4a^{D249N} and end-stage of Tubb4a^{D249N/D249N} in cerebellum. Scale bar=1 mm (FIG. 6F) Representative P21 images of PLP (green) of WT, Tubb4a^{D249N} and Tubb4a^{D249N/D249N} mice in cerebellum. Scale bar=1 mm (FIG. 6G) Representative P21 images of MBP (red) of WT, Tubb4a^{D249N} and Tubb4a^{D249N/D249N} mice in cerebellum. Scale bar=250 μm

FIGS. 7A-7J: Tubb4a^{D249N/D249N} mice display reduced number of oligodendrocytes (OL) (FIG. 7A) Schematic diagram displaying the time course of immunohistochemical assays. (FIG. 7B) Schematic diagram showing area of corpus callosum used to perform counts. (FIG. 7C) Representative images of ASPA positive OL in WT, Tubb4a^{D249N} and Tubb4a^{D249N/D249N} mice at the end-stage (ES). Scale bar=50 μm and 25 μm. (FIG. 7D) Quantification of ASPA positive OL counts/mm² at P14, P21 and ES in WT, Tubb4a^{D249N} and Tubb4a^{D249N/D249N} mice. (FIG. 7E) Representative images of double positive NG2+ Olig2+ in WT, Tubb4a^{D249N} and Tubb4a^{D249N/D249N} mice at ES. Scale bar=50 μm and 25 μm (FIG. 7F) Quantification of double positive NG2+ Olig2+ counts/mm² at P14, P21 and ES in WT, Tubb4a^{D249N} and Tubb4a^{D249N/D249N} mice (FIG. 7G) Representative data of two independent experiments with n=4 mice/group for P14 (except n=3 for Tubb4a^{D249N} for P14 time point) and P21 and n=3 mice/group for ES. (FIG. 7H) Quantification of double-positive NG2+ caspase cells/mm2 at P14, P21, and ˜P35-P40 of WT, Tubb4aD249N/+, and ES Tubb4aD249N/D249N mice. (FIG. 7I) Representative images of double-positive NG2+Ki-67 cells in WT, Tubb4aD249N/+, and Tubb4a^{D249N/D249N} mice at ES. (FIG. 7J) Quantification of double-positive NG2+Ki-67 cells/mm2 at P14, P21, and ˜P35-P40 of WT, Tubb4aD249N/+, and ES Tubb4a^{D249N/D249N} mice. Statistical test is performed by two-way ANOVA, followed by Tukey post-hoc test. Data is presented as mean and SEM. *p<0.05 and ***p<0.001.

FIGS. 8A-8G: Tubb4a^{D249/D249N} mice show hypomyelination at P14 (FIG. 8A) Schematic diagram displaying the time course of immunohistochemical assays. (FIG. 8B) Representative P14 images of Eriochrome cyanine (Eri-C) stain (myelin [blue]) of WT, Tubb4a^{D249N} and Tubb4a^{D249N/D249N} in corpus callosum. Scale bar=1 mm (FIG. 8C) Representative P14 images of PLP (green) of WT, Tubb4a^{D249N} and Tubb4a^{D249N/D249N} mice in corpus callosum. Scale bar=250 μm (FIG. 8D) Representative P14 images of MBP (red) of WT, Tubb4a^{D249N} and Tubb4a^{D249N/D249N} mice in corpus callosum. Scale bar=250 μm (FIG. 8E) Representative P14 images of Eriochrome cyanine (Eri-C) stain (myelin [blue]) of WT, Tubb4a^{D249N} and end-stage of Tubb4a^{D249N/D249N} in cerebellum. Scale bar=1 mm (FIG. 8F) Representative P14 images of PLP (green) of WT, Tubb4a^{D249N} and Tubb4a^{D249N/D249N} mice in cerebellum. Scale bar=1 mm (FIG. 8G) Representative P14 images of MBP (red) of WT, Tubb4a^{D249N} and Tubb4a^{D249N/D249N} mice in cerebellum. Scale bar=250 μm FIGS. 9A-9J: Tubb4a^{D249N/D249N} mice show severe cerebellar granular neuronal loss and significant striatal neuronal loss (FIG. 9A) Schematic diagram displaying the time course of immunohistochemical assays. (FIG. 9B) Schematic diagram showing the whole brain mounts of WT, Tubb4a^{D249N} and Tubb4a^{D249N/D249N} mice at P40. (FIG. 9C) Nissl stain images of cerebellum at P21 and P40 of WT and Tubb4a^{D249N/D249N} mice. Scale bar=1 mm (FIG. 9D) Representative images of NeuN (green) showing cerebellar granular neurons at P21 and End-stage (ES) of WT and Tubb4a^{D249N/D249N} mice. Scale bar=1 mm (FIG. 9E) Quantification of cerebellar granular neuron counts/mm² at P14, P21 and ES. (FIG. 9F) Representative images of double immuno-positive cerebellar granule neurons stained by NeuN (green) and cleaved caspase 3 (red) (as shown by white arrow) at P21 and ES of WT and Tubb4a^{D249N/D249N} mice. Scale bar=25 μm (FIG. 9H) Schematic diagram of Striatum showing area used for quantifying neuronal counts (by dashed box). Scale bar=1 mm (FIG. 9I) Representative images of striatal neurons stained by NeuN (green) at ES of WT, Tubb4a^{D249N} and Tubb4a^{D249N/D249N} mice. Scale bar=100 μm (FIG. 9J) Quantification of striatal neuronal counts/mm² at P14, P21 and ES in WT, Tubb4a^{D249N} and Tubb4a^{D249N/D249N} mice. Statistical test is performed by two-way ANOVA, followed by Tukey post-hoc test. Representative data of two independent experiments with n=4 mice/group for P14 and P21 (except n=3 for Tubb4a^{D249N} for P14 time point) and n=3 mice/group for ES. Data is presented as mean and SEM. *p<0.05 and ***p<0.001.

FIGS. 10A-10C: Tubb4a^{D249/D249N} mice show hypomyelination Representative Eri-C (myelin) stain images of sagittal sections of WT and Tubb4a^{D249N/D249N} at P14 (FIG. 10A), P21 (FIG. 10B) and ES (FIG. 10C) (Scale bar=1 mm).

FIGS. 11A-11K: Oligodendrocytes and neurons from Tubb4a^{D249N} and Tubb4a^{D249/D249N} mice display reduced branching and processes (FIGS. 11A-C) Representative images of PLP labeled oligodendrocytes (OL) isolated from WT, Tubb4a^{D249N} and Tubb4a^{D249/D249N} mice. Scale bar=50 μm (FIG. 11D) The number of Olig2 labeled cells were counted in coverslips from WT, Tubb4a^{D249N} and Tubb4a^{D249/D249N} mice and plotted as percentage of Olig2+ cells in WT animals. (FIG. 11E) Total number of PLP+ cells quantified from WT, Tubb4a^{D249N} and Tubb4a^{D249/D249N} mice were plotted as a percentage of PLP+ cells in WT animals. (FIG. 11F) Number of mature PLP+ cells from the total Olig2+ cells were plotted as a percent of WT animals. The experiments were repeated at least three independent times. (FIG. 11G) Representative images of cortical neurons from WT mice stained with Tuj1 (axonal marker) and MAP2 (dendritic marker). (FIG. 11H) Representative images of cortical neurons from Tubb4a^{D249/D249N} mice stained with Tuj1 and MAP2. Scale bar=75 μm (FIG. 11I) Number of surviving neurons at 1-week post-plating were quantified and plotted as percent of WT neurons (FIG. 11J) Axon length was measured using Neurite tracer plugin and plotted for all groups (FIG. 11K) Dendritic length was measured using Neurite tracer plugin and plotted for all groups. Data is presented as mean and SEM. One-way ANOVA was performed on the data set followed by Tukey post-hoc test. *p<0.05, ***p<0.001

FIGS. 12A-12E: Tubb4a^{D249/D249N} mice show reduced number of oligodendrocytes at P14 and P21 (FIGS. 12A-B) Representative images of ASPA positive OL in WT, Tubb4a^{D249N} and Tubb4a^{D249N/D249N} mice at the P14 (FIG. 12A) and P21 (FIG. 12B). Scale bar=50 μm and 25 μm (FIGS. 12C-12D) Representative images of double positive NG+ Olig2+ in WT, Tubb4a^{D249N} and Tubb4a^{D249N/D249N} mice at the P14 (FIG. 12C) and P21 (FIG. 12D). Scale bar=50 μm and 25 μm (FIG. 12E) Quantification of counts of Olig2+ cells at P14, P21 and ES. Statistical test is performed by two-way ANOVA, followed by Tukey post-hoc test. Data is presented as mean and SEM.

FIGS. 13A-13F: Microtubule polymerization is affected in Tubb4a^{D249N} and Tubb4a^{D249/D249N} mice (FIG. 13A) Example kymographs generated based on EB3 tracking from WT, Tubb4a^{D249N} and Tubb4a^{D249/D249N} cortical neurons. (FIG. 13B) Graph for number of EB3 comets per 100 μm followed for a period of 10 mins are plotted. (FIG. 13C) Velocity plotted for WT, Tubb4a^{D249N} and Tubb4a^{D249/D249N} microtubule polymerization based on EB3 trafficking. (FIGS. 13D-E) Total run time (FIG. 13D) and histogram of run time (FIG. 13E) plotted for WT, Tubb4a^{D249N} and Tubb4a^{D249/D249N} EB3 comets. (FIG. 13F) Total run length plotted for WT, Tubb4a^{D249N} and Tubb4a^{D249/D249N} EB3 comets. Data is presented as mean and SEM. One-way ANOVA was performed on the data set followed by Tukey post-hoc test. *p<0.05, **p<0.001, ***p<0.001.

FIGS. 14A-14E: Tubb4a^{D249/D249N} mice show comparable Purkinje neuronal counts (FIG. 14A) Representative images of cerebellar granule neurons and caspase staining at P14 of WT, Tubb4a^{D249N} and Tubb4a^{D249N/D249N} mice. Scale bar=1 mm and 50 μm (FIG. 14B) Representative images of Striatal neurons at P14 and P21 of WT, Tubb4a^{D249N} and Tubb4a^{D249N/D249N} mice. Scale bar=100 μm (FIG. 14C) Nissl staining at P14 of WT and Tubb4a^{D249N/D249N} mice. Scale bar=1 mm (FIG. 14D) Representative images of Purkinje neurons stained by calbindin at P21 and end-stage of WT, Tubb4a^{D249N} and Tubb4a^{D249N/D249N} mice. Scale bar=100 μm (FIG. 14E) Quantification of Purkinje neuronal counts/mm² at P21 and end-stage of WT, Tubb4a^{D249N} and Tubb4a^{D249N/D249N} mice. Statistical test is performed by two-way ANOVA, followed by Tukey post-hoc test. Data is presented as mean and SEM.

FIGS. 15A-15B: Tubb4a^{D249/D249N} mice show OL cell death at P14 and P21 (FIG. 15A) Representative images of double positive Olig2+ Caspase in WT, Tubb4a^{D249N} and Tubb4a^{D249N/D249N} mice at P14. (FIG. 15B) Representative images of double positive Olig2+ Caspase in WT, Tubb4a^{D249N} and Tubb4a^{D249N/D249N} mice at P21. Scale bar=50 μm and 25 μm FIGS. 16A-16D: Map2 and Dapi staining of wild type (FIG. 16A) and Tubb4a^{D249/D249N} (FIG. 16B) cells. Total neurons (FIG. 16C) and striatal neurons (FIG. 16D, CTIP2+ neurons) from TUBB4A^{D249N} iPSC derived neurons have decreased survival in comparison to control iPSC derived neurons and (FIG. 16E) TUBB4A^{D249N} have increased apoptosis and neuropathology. (FIGS. 16F-G) CRISPR mediated deletion of TUBB4A in control patient line doesn't affect development/formation of neurons. *p<0.05, **p<0.01, ***p<0.001

FIGS. 17A-17B: Deletion of TUBB4A is neuroprotective in TUBB4^{AD249N} MSNs Quantification of total neurons (FIG. 17A) and medium spiny neurons (FIG. 17B) TUBB4A^{D249N} MSNs.

FIGS. 18A-18C: (FIG. 18A) Diagram of ASO electroporation (FIG. 18B) Diagram of ASO working principle (FIG. 18C) Selection of two promising ASOs after screening of ASO library. ***p<0.0001.

FIG. 19: Schematic of in vivo whole animal model and treatment protocol for H-ABC.

FIGS. 20A-20C: Therapeutic efficacy of downregulating TUBB4A in established Tubb4a^{D249N/D249N} mouse model. Established mouse model was crossed with the Tubb4a knock out (KO) mice, which are viable and appear normal. (FIG. 20A) Diagram of Tubb4a transgenic mouse crosses. (FIG. 20B) The resulting Tubb4a^{D249N}/KO mice display improved motor function and increased survival relative to Tubb4a^{D249N/D249N} mice, with extended survival of Tubb4a^{D249N}/KO (˜P108) compared to Tubb4a^{D249N/D249N} mice (˜P35-P40). (FIG. 20C). Improved motor function based on rotarod performance of Tubb4a^{D249N}/KO versus Tubb4a^{D249N/D249N} (n=23 ***p<0.0001).

FIGS. 21A-21D: Evaluation of ASOs as a viable therapeutic target. A library of ASOs were screened using mouse Oli-neu cells. Two potent ASOs (ASO 1316 and 1851) show maximal Tubb4a knockdown. (FIG. 21A) Selection of two promising ASOs after screening of ASO library in vitro by qRT-PCR in Oli-Neu cells. ***p<0.0001. Administration of a single intracereberoventricular (ICV) bolus injection with variable ASO doses (25, 10, 5, 2, 1 and 0.5 μg/g of dose) established the dose-response. The most potent ASO 1316 with least toxicity was selected (data not shown). (FIG. 21B) A single ICV injection at 2 μg/g dose in P1 Tubb4a^{D249N/D249N} mice, demonstrates increased survival in treated mice as compared to control mice (PBS, scrambled ASO). (FIG. 21C) Tubb4a^{D249N/D249N} mice treated with Tubb4a ASO also exhibit reduced seizures from P34-P37 as compared to control mice (PBS, scrambled ASO). (FIG. 21D) Further, these mice display significant improvement in motor function as measured by rotarod at P28 and P35 (NC5-Scrambled ASOs; *p<0.01, **p<0.001, *** p<0.0001). FIG. 22: Overexpression of GFP-WT-Tubb4a in cultured Oli-Neu cells resulted in increased MBP, PLP and CNP mRNA levels as seen in qRT-PCR (n=4-6, ***p<0.05). (Mock—Vehicle control)

### DETAILED DESCRIPTION OF THE INVENTION

Hypomyelination and atrophy of basal ganglia and cerebellum (H-ABC) is a rare hypomyelinating leukodystrophy associated with causal variants in tubulin alpha 4 (TUBB4A); p.Asp249Asn (D249N) is a recurring variant occurring in the majority of affected individuals. Monoallelic mutations in TUBB4A may also result in a larger spectrum of neurologic disorders ranging from an early onset encephalopathy to an adult-onset Dystonia type 4 (whispering dysphonia). H-ABC is within this spectrum, and typically begins in early childhood characterized by dystonia, ataxia, altered gait and progressive motor dysfunction with loss of ambulation before the end of the first decade of life. To date, there is no therapeutic approach available for this progressive and disabling pediatric disorder. To understand how TUBB4A mutations cause H-ABC and to facilitate the development and pre-clinical testing of therapeutic strategies, our group has developed a knock-in mouse model harboring heterozygous (Tubb4a^{D249N}) or homozygous (Tubb4a^{D249N/D249N}) Tubb4a mutations using a CRISPR-Cas9 approach.

We now provide the first mouse model of classical H-ABC (Tubb4a^{D249N/D249N}), which displays decreased survival, progressive motor dysfunction with tremor, abnormal gait and ataxia, thus recapitulating the phenotypic features of the disease. Neuropathological assessment of Tubb4a^{D249N/D249N} mice using immunolabeling and western blot on post-natal day (P) 14, P21 and end-stage P40 shows initial delay of myelination followed by ultimate demyelination. There is decrease of myelin proteins over time and dramatic loss of ASPA positive oligodendrocytes (myelinating cells in CNS). Ultrathin brain sections for electron microscopy further demonstrated hypomyelination and ongoing loss of myelin in spinal cord and optic nerves of these mice. In addition, in vitro studies on oligodendrocytes in culture from Tubb4a^{D249N/D249N} mice demonstrated decreased maturation and myelin markers. Similarly, neuropathology demonstrates severe neuronal loss in the striatum and cerebellar granule cells. Further, effects in neurons in culture were noted with decreased neuronal survival along with unstable microtubule dynamics in cells from Tubb4a^{D249N/D249N} mice. Tubb4a^{D249N/D249N} mice provide a novel mouse model for H-ABC, and demonstrate the complexity of cellular physiology in this disorder, with potential microtubule instability from TUBB4A mutations, resulting in cell autonomous effects on oligodendrocytes, striatal neurons and cerebellar granule cells, and profound neurodevelopmental phenotypes.

In additional studies, we provide reprogrammed, induced, pluripotent stem cell lines from peripheral blood samples obtained from H-ABC patients. Use of these cell lines has revealed new therapeutic paradigms for the treatment of leukodystrophy by downmodulating the mutant Tubb-4a encoding nucleic acid using nucleic acids which target this sequence. In alternative approaches, vectors are provided with overexpress wild-type Tubb-4a at sites of interest. Overexpression of Tubb-4a is associated with increased MBP, PLP, and CNP mRNA levels, and should alleviate H-ABC symptoms in subjects in need of such treatment.

We have also developed new therapeutic antisense oligonucleotides which effectively down modulate TUBB4A expression, thereby providing a new approach for treatment of leukodystrophy.

### Definitions

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action. The terms “agent” and “test compound” denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the TUBB4A containing nucleic acids described herein or their encoded proteins.

As used herein, “TUBB4A” refers to a gene which encodes a member of the beta tubulin family. Beta tubulins are one of two core protein families (alpha and beta tubulins) that heterodimerize and assemble to form microtubules. Mutations in this gene cause hypomyelinating leukodystrophy-6 and autosomal dominant torsion dystonia-4 and H-ABC, now more commonly referred to as TUBB4-A-Associated Leukoencephalopathy. Reference sequences for TUBB4A include for example NM_001289123.1. NM_001289127.1 NM_001289129.1 which can be found on GenBank. Alternate splicing results in multiple transcript variants encoding different isoforms. The wild type Tubb4a protein sequence is found on UniProt, accession no. P04350-TBB4A_human. Several TUBB4A variants known to be associated with human disease have been identified and are listed below in Table 1. The present invention focuses on the D249N variant, however the findings are generalizable to other existing TUBB4A mutations.

TABLE 1
TUBB4A Variants

c.76A>T
c.1052C>T
c.1054G>A
c.1061G>A
c.1062C>G
c.1088T>C
c.1099T>A
c.1099T>C
c.1162A>G
c.1164G>A
c.1164G>T
c.1171C>T
c.1172G>A
c.1172G>T
c.1181T>G
c.1190G>T
c.1228G>A
c.1254G>T
c.1325G>A
c.286G>A
c.293G>A
c.395G>C
c.467G>T
c.4C>G
c.4C>T
c.518A>T
c.523G>A
c.533C>A
c.533C>G
c.533C>T
c.535G>A
c.535G>C
c.535G>T
c.538G>A
c.539A>C
c.568C>T
c.5G>A
c.691G>A
c.716G>A
c.716G>T
c.730G>A
c.730G>C
c.730G>T
c.731G>A
c.731G>C
c.731G>T
c.743C>A
c.745G>A
c.755A>G
c.763G>A
c.785G>A
c.796T>A
c.811G>A
c.845G>C
c.874C>A
c.916G>A
c.968T>G
c.971A>C

Table 2 provides a listing of certain amino acid changes associated with different forms of TUBB4A-Associated Leukoencephalopathy.

TABLE 2

| | Human | Cell Type |
|---|---|---|
| Mutation | Phenotype | Affected |
| p.Arg2G1y | Whispering | Neurons |
| (R2G) | Dysphonia | |
| pAsp249Arg | Classical | Neurons and |
| (D249N) | H-ABC | oligodendrocytes |
| p.Arg28Pro | Isolated | oligodendrocytes |
| (R282P) | hypomyelination | |
| pArg391His | Isolated | oligodendrocytes |
| (R391H) | hypomyelination | |

As used herein, the terms “treatment” or ‘therapy’ (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Several aspects of the invention relate to vector systems comprising one or more vectors, or vectors as such. Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press. San Diego, Calif. (1990). Alternatively, the recombinant expression vector can be transcribed and translated in vitro, for example using T7 promoter regulatory sequences and T7 polymerase.

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). In order to obtain high levels of expression, the Tubb4-A encoding nucleic acid can be codon-optimized.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell. In alternate embodiments, the CRISPR-mediated base editor is base editor 4 (BE4) rather than BE3. Notably, base editing can be employed to alter any of the mutated nucleic acids listed in Table 1.

In some embodiments, the method further comprises assessing C bases within the window for a change to another base. In embodiments, the gRNA is selected if the BE3 PAM sequence (NGG) is 13-17 nucleotides distal to the target cytosine base(s). In particular embodiments, the change is via a C to T on a sense strand, and the modified codon is changed to a nonsense codon. In additional embodiments, the change is via a G to A on an antisense strand, and the modified codon is changed to a nonsense codon. In further embodiments, the change is via a C to T on a sense strand, and the change is a missense variant. In additional embodiments, the change is via a G to A on an antisense strand, and the change is a missense variant. The base editing may occur prior to disease onset, wherein the disease is a phenotype resulting from a mutation in the therapeutic gene. In certain embodiments, the base editing decreases a risk of developing a disease.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

In some aspects, the invention provides methods comprising delivering one or more polynucleotides (e.g., a CRISPR system, an antisense oligonucleotide, an siRNA, an shRNA, a triplex nucleic acid, etc), such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a CRISPR enzyme in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system.

Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and Y2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line.

In one aspect, the invention provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may be re-introduced into the human or non-human animal.

In one aspect, the invention provides for methods of modifying a target polynucleotide in a eukaryotic cell. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence.

In one aspect, the invention provides kits containing any one or more of the elements disclosed in the above methods and compositions. In some embodiments, the kit comprises a vector system or components for an alternative delivery system such as those described above and instructions for using the kit. In some embodiments, the vector or delivery system comprises (a) a first regulatory element operably linked to a tracr mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and/or (b) a second regulatory element operably linked to an enzyme-coding sequence encoding said CRISPR enzyme comprising a nuclear localization sequence. Elements may be provided individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

Down-modulating or inhibitory nucleic acids include, without limitation, antisense molecules, aptamers, ribozymes, triplex forming molecules, RNA interference (RNAi), CRISPR (Clustered Regularly Interspaced Short Palindromic Repeats) RNA (crRNA), and external guide sequences. These nucleic acid molecules can act as affectors, inhibitors, modulators, and stimulators of a specific activity possessed by a target molecule, or the functional nucleic acid molecules can possess a de novo activity independent of any other molecules. In certain embodiments, inhibitory nucleic acids are employed.

Antisense molecules are designed to interact with a target nucleic acid molecule through either canonical or non-canonical base pairing. The interaction of the antisense molecule and the target molecule is designed to promote the destruction of the target molecule through, for example, RNase H mediated RNA-DNA hybrid degradation. Alternatively, the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹². A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹². Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426. Gene expression can also be effectively silenced in a highly specific manner through RNA interference (RNAi). This silencing was originally observed with the addition of double stranded RNA (dsRNA) (Fire, A., et al., Nature, 391:806-11 (1998); Napoli, C., et al., Plant Cell, 2:279-89 (1990); Hannon, G. J., Nature, 418:244-51 (2002)). Once dsRNA enters a cell, it is cleaved by an RNase III-like enzyme, Dicer, into double stranded small interfering RNAs (siRNA) 21-23 nucleotides in length that contain 2 nucleotide overhangs on the 3′ ends (Elbashir, S. M., et al., Genes Dev., 15:188-200 (2001); Bernstein, E., et al., Nature, 409:363-6 (2001); Hammond, S. M., et al., Nature, 404:293-6 (2000)). In an ATP-dependent step, the siRNAs become integrated into a multi-subunit protein complex, commonly known as the RNAi induced silencing complex (RISC), which guides the siRNAs to the target RNA sequence (Nykanen, A., et al., Cell, 107:309-21 (2001)). At some point the siRNA duplex unwinds, and it appears that the antisense strand remains bound to RISC and directs degradation of the complementary mRNA sequence by a combination of endo and exonucleases (Martinez, J., et al., Cell, 110:563-74 (2002)). However, the effect of RNAi or siRNA or their use is not limited to any type of mechanism.

Small Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al., Nature, 411:494 498(2001); Ui-Tei, K., et al., FEBS Lett, 479:79-82 (2000)). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

Similar to RNAi, CRISPR (Clustered Regularly Interspaced Short Palindromic Repeats) interference is a powerful approach, via selective DNA cleavage, for reducing gene expression of endogenously expressed proteins. CRISPRs are genetic elements containing direct repeats separated by unique spacers, many of which are identical to sequences found in phage and other foreign genetic elements. Recent work has demonstrated the role of CRISPRs in adaptive immunity and shown that small RNAs derived from CRISPRs (crRNAs) are implemented as homing oligonucleotides for the targeted interference of foreign DNA (Jinek et al., Science, 337:816-821 (2012)). crRNAs are used to selectively cleave DNA at the genetic level.

The term shRNA refers to short hairpin RNA, an RNA structure that forms a tight hairpin turn, which can also be used to silence gene expression via RNA interference. The shRNA hairpin structure is cleaved by the cellular machinery into small interfering RNA (siRNA), which is then bound to the RNA-induced silencing complex (RISC). This complex binds to and cleaves mRNA, which matches the siRNA that is bound to it.

As used herein, the term “overexpressing” when referring to the production of a protein in a host cell means that the protein is produced in greater amounts than it is produced in its naturally occurring environment.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

“Target nucleic acid” as used herein refers to a previously defined region of a nucleic acid present in a complex nucleic acid mixture wherein the defined wild-type region contains at least one known nucleotide variation associated with leukodystrophy. The nucleic acid molecule may be isolated from a natural source by cDNA cloning or subtractive hybridization or synthesized manually. The nucleic acid molecule may be synthesized manually by the triester synthetic method or by using an automated DNA synthesizer.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the Leukodystrophy specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the leukodystrophy specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

As mentioned above, the introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The phrase “modified backbone linkage”, includes but is not limited to phosphorothioate linkages, methylphosphonate linkages, ethylphosphonate linkages, boranophosphate linkages, sulfonamide, carbonylamide, phosphorodiamidate, phosphorodiamidate linkages comprising a positively charged side group, phosphorodithioates, aminoethylglycine, phosphotriesters, aminoalkylphosphotriesters; 3′-alkylene phosphonates; 5′-alkylene phosphonates, chiral phosphonates, phosphinates, 3′-amino phosphoramidate, aminoalkylphosphoramidates, thionophosphoramidates; thionoalkyl-phosphonates, thionoalkylphosphotriesters, selenophosphates, 2-5′ linked boranophosphonate analogs, linkages having inverted polarity, abasic linkages, short chain alkyl linkages, cycloalkyl internucleoside linkages, mixed heteroatom and alkyl or cycloalkyl internucleoside linkages, short chain heteroatomic or heterocyclic internucleoside linkages with siloxane backbones, sulfide, sulfoxide, sulfone, formacetyl linkages, thioformacetyl linkages, methylene formacetyl linkages, thioformacetyl linkages, riboacetyl linkages, alkene linkages, sulfamate backbones, methyleneimino linkages, methylenehydrazino linkages, sulfonate linkages, and amide linkages.

The phrase “modified sugar” includes, without limitation, 2′ fluoro, 2′ fluoro substituted ribose, 2′-fluoro-D-arabinonucleic acid (FANA), 2′-O-methoxyethyl ribose, 2′-O-methoxyethyl deoxyribose, 2′-O-methyl substituted ribose, a morpholino, a piperazine, and a locked nucleic acid (LNA).

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample which may be tested for a particular molecule, preferably an leukodystrophy specific marker molecule, such as a marker shown in the tables provided below. Samples may include but are not limited to cells, body fluids, including blood, serum, plasma, urine, saliva, cerebral spinal fluid, tears, pleural fluid and the like.

### Kits and Articles of Manufacture

Any of the aforementioned products can be incorporated into a kit which may contain a TUBB-4A directed down modulating nucleic acids in pharmaceutically acceptable carrier. The nucleic acid may or may not be disposed in a vector which is capable of transducing mammalian cells. In other aspects, the kit contains a vector which expresses human wild type TUBB-4A and/or mutant TUBB4A encoding nucleic acids for overexpressing the same in target cells of interest. The kit may optionally include nanoparticle or liposome formulations which facilitate delivery of the nucleic acids into cells. The kit may also contain instructions for use, a container, a vessel for administration, an assay substrate, or any combination thereof.

### Methods for the Development and Screening of Therapeutic Agents

Since genetic alterations in TUBB4-A identified herein have been associated with the etiology of H-ABC, methods for identifying agents that modulate the activity of the mutated genes and their encoded products should result in the generation of efficacious therapeutic agents for the treatment of leukodystrophy, particularly H-ABC.

Molecular modeling should facilitate the identification of specific organic molecules with capacity to bind to the active site of altered TUBB4-A proteins based on conformation or key amino acid residues required for function. A combinatorial chemistry approach will be used to identify molecules with greatest activity and then iterations of these molecules will be developed for further cycles of screening.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered TUBB4-A associated gene. These host cell lines or cells are defective at the polypeptide level. The host cell lines or cells are grown in the presence of drug compound. The rate of cellular metabolism of the host cells is measured to determine if the compound is capable of regulating the cellular metabolism in the defective cells. Methods for introducing DNA molecules are also well known to those of ordinary skill in the art as discussed above.

Host cells expressing the H-ABC associated nucleic acids of the present invention or functional fragments thereof provide a system in which to screen potential compounds or agents for the ability to modulate the development of leukodystrophy. Thus, in one embodiment, the nucleic acid molecules of the invention may be used to create recombinant cell lines for use in assays to identify agents which modulate aspects of cellular metabolism associated with neuronal signaling and neuronal cell communication and structure. Also provided herein are methods to screen for compounds capable of modulating the function of proteins encoded by TUBB4-A containing nucleic acids.

Another approach entails the use of phage display libraries engineered to express fragment of the polypeptides encoded by the altered TUBB4-A nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays. Such compound libraries are commercially available from a number of companies including but not limited to Maybridge Chemical Co., (Trevillet, Cornwall, UK), Comgenex (Princeton, N.J.), Microsour (New Milford, Conn.) Aldrich (Milwaukee, Wis.) Akos Consulting and Solutions GmbH (Basel, Switzerland), Ambinter (Paris, France), Asinex (Moscow, Russia) Aurora (Graz, Austria), BioFocus DPI (Switzerland), Bionet (Camelford, UK), Chembridge (San Diego, Calif.), Chem Div (San Diego, Calif.). The skilled person is aware of other sources and can readily purchase the same. Once therapeutically efficacious compounds are identified in the screening assays described herein, they can be formulated into pharmaceutical compositions and utilized for the treatment of H-ABC.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

In another embodiment, the availability of altered TUBB-4A nucleic acids enables the production of strains of laboratory mice carrying the leukodystrophy-associated TUBB4-A nucleic acids of the invention. Transgenic mice expressing the leukodystrophy-associated nucleic acids of the invention provide a model system in which to examine the role of the mutated Tubb4-a protein encoded by the g nucleic acid in the development and progression towards leukodystrophy. Methods of introducing transgenes in laboratory mice are known to those of skill in the art and are described hereinbelow. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that a target protein plays in various cellular metabolic and neuronal processes. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

Techniques are available to inactivate or alter any genetic region to a mutation desired by As used herein, a knock-in animal is one in which the endogenous murine gene, for example, has been replaced with human leukodystrophy-associated TUBB4-A gene of the invention. Such knock-in animals provide an ideal model system for studying the development of leukodystrophy. Knock-out animals can also be created.

As used herein, the expression of a leukodystrophy associated nucleic acid, fragment thereof, can be targeted in a “tissue specific manner” or “cell type specific manner” using a vector in which nucleic acid sequences encoding all or a portion of leukodystrophy-associated nucleic acids are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein.

Methods of use for the transgenic mice of the invention are also provided herein. Transgenic mice into which a nucleic acid containing the leukodystrophy-associated TUBB4-A or its encoded protein have been introduced are useful, for example, to develop screening methods to screen therapeutic agents to identify those capable of modulating the development of leukodystrophy.

### Pharmaceuticals and Peptide Therapies

The elucidation of the role played by the leukodystrophy associated CNVs/SNPs described herein in neuronal signaling and brain structure facilitates the development of pharmaceutical compositions useful for treatment and diagnosis of leukodystrophy. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

### Pharmaceutical Compositions

Pharmaceutical compositions containing a therapeutic, prophylactic, or diagnostic agent derivative, such as functional nucleic acid derivative, may be administered parenterally to subjects in need of such a treatment. Parenteral administration can be performed by subcutaneous, intramuscular or intravenous injection by means of a syringe, optionally a pen-like syringe. Alternatively, parenteral administration can be performed by means of an infusion pump. Further options are to administer the therapeutic, prophylactic, or diagnostic agent nasally or pulmonally, preferably in compositions, powders or liquids, specifically designed for the purpose.

Injectable compositions of the therapeutic, prophylactic, or diagnostic agent derivatives can be prepared using the conventional techniques of the pharmaceutical industry which involve dissolving and mixing the ingredients as appropriate to give the desired end product. Thus, according to one procedure, a therapeutic, prophylactic, or diagnostic agent derivative can be dissolved in an amount of water which is somewhat less than the final volume of the composition to be prepared. An isotonic agent, a preservative and a buffer can be added as required and the pH value of the solution is adjusted—if necessary—using an acid, e.g., hydrochloric acid, or a base, e.g., aqueous sodium hydroxide, as needed. Finally, the volume of the solution can be adjusted with water to give the desired concentration of the ingredients.

In some embodiments, the buffer can be selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers and their combinations constitutes an alternative embodiment.

The following materials and methods are provided to facilitate the practice of the present invention.

### Generation of Mouse Model

Heterozygous Tubb4a^{D249N} mice were generated using clustered regularly interspaced short palindromic repeats (CRISPR)—Cas-9 technology by inserting the p.Asp249Asn (c.745G>A) mutation in exon 4 of the Tubb4a gene. The mouse Tubb4a gene is located on Chromosome 17 comprising of 4 exons. Cas9 mRNA, gRNA and oligonucleotides (with targeting sequence, flanked by 120 bp homologous combined on both sides) were co-injected into zygotes. The resulting CRISPR knock-in mouse model has the heterozygous point mutation of c.745G>A in one allele of the Tubb4a gene (Tubb4a^{D249N}). These heterozygous mice were bred to produce homozygous Tubb4a^{D249N/D249N} mice in keeping with the homozygous mutation seen in taeip rat models (Li et al., 2003) in addition to heterozygous Tubb4a^{D249N} animals. Wild-type (WT), Tubb4a^{D249N} and Tubb4a^{D249N/D249N} mice were included in all analyses. The animals were genotyped at all experimental steps. Mice were maintained under a 12 h light:12 h dark cycle in a clean facility and given free access to food and water. The methods and study protocols were approved in full by the Institutional Animal Care and Use Committee of the Children's hospital of Philadelphia and conformed with the revised National Institutes of Health Office of Laboratory Animal Welfare Policy.

### Behavioral Analysis

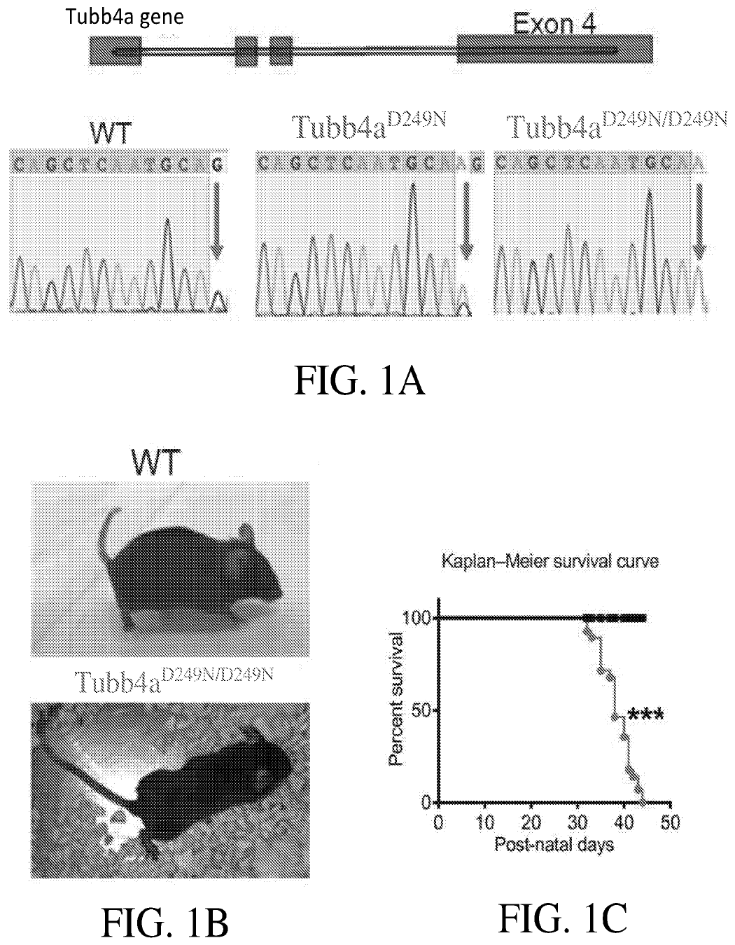

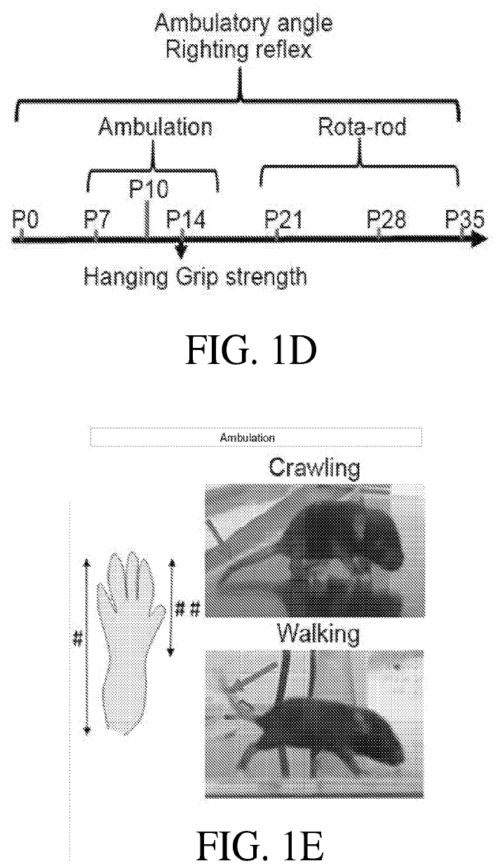

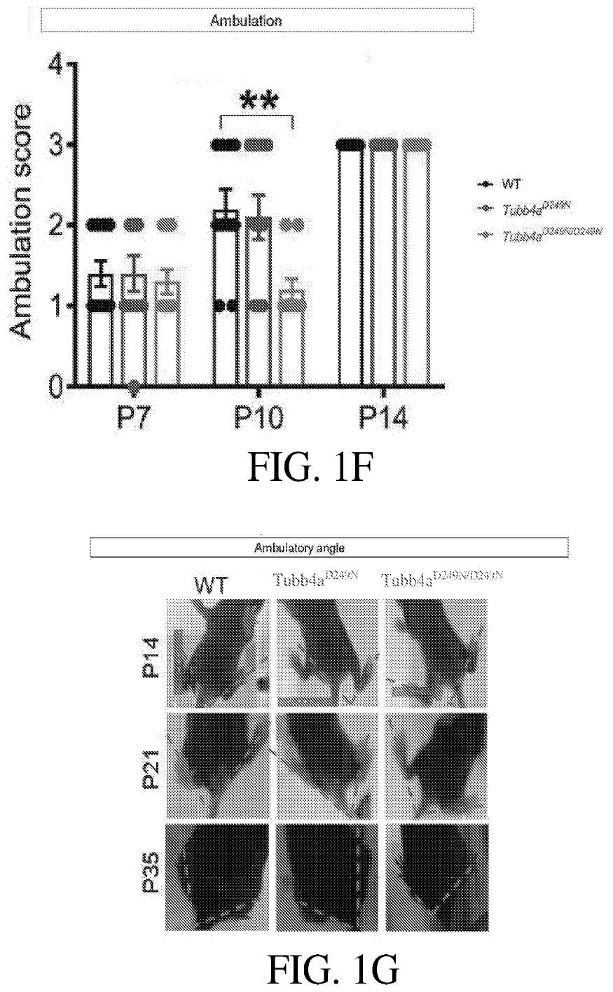

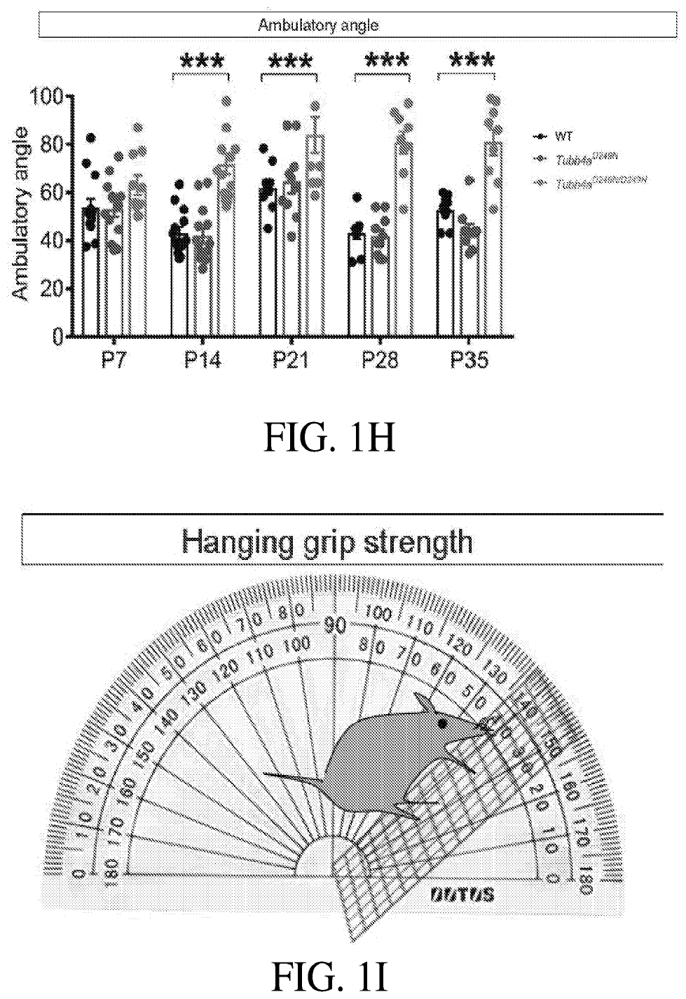

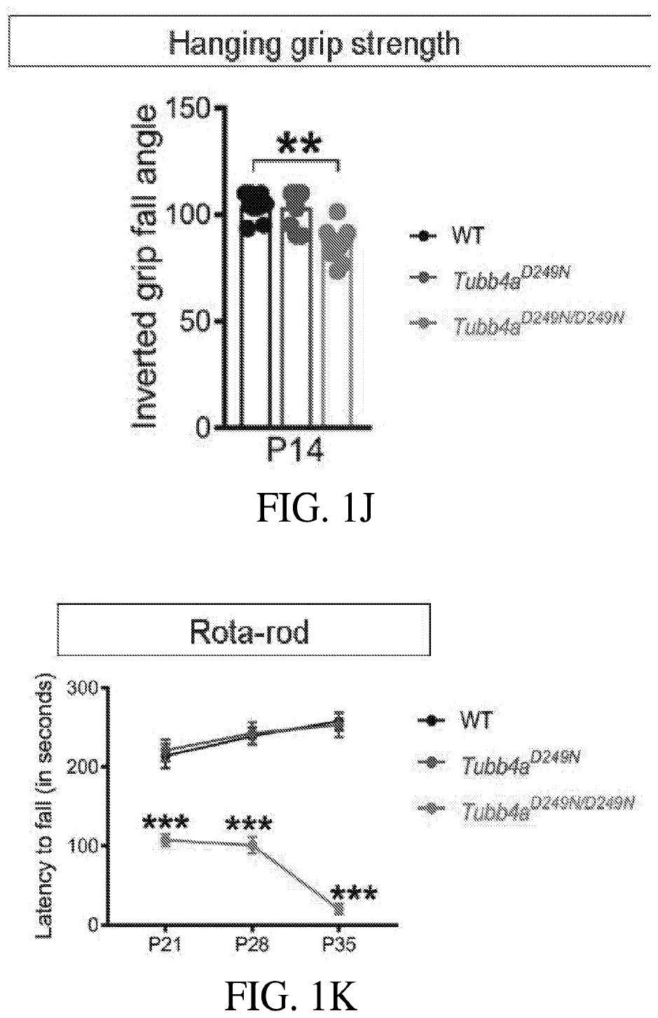

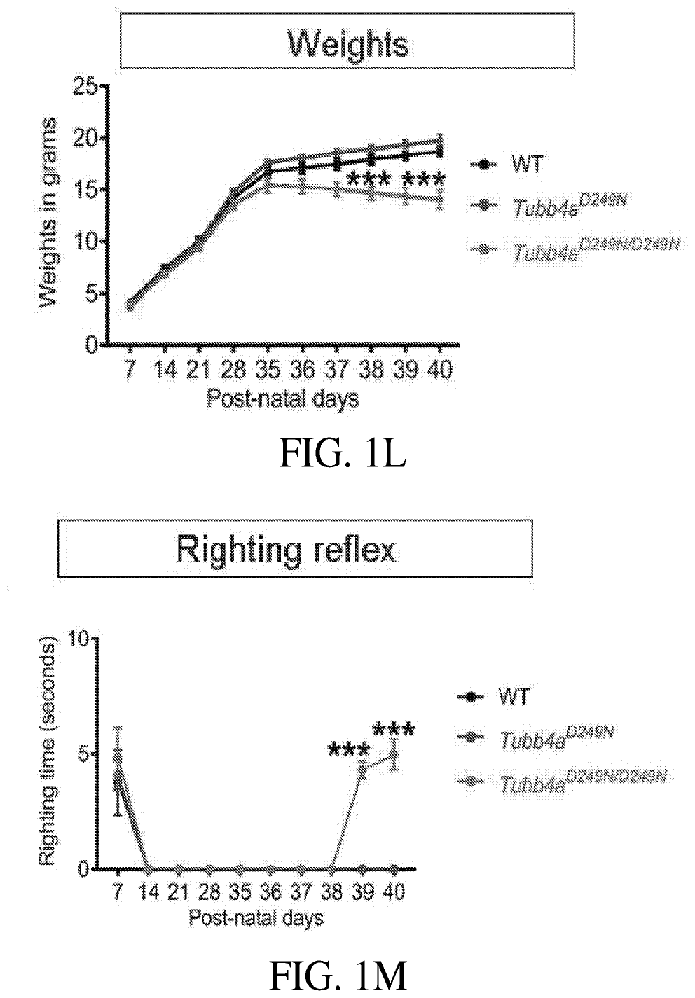

Ambulatory angle, ambulation, hanging grip strength, righting reflex (Feather-Schussler and Ferguson, 2016) and Rota Rod (Shiotsuki et al., 2010) were assessed at defined developmental intervals (FIG. 1). At least a cohort of 10 animals per condition were included for the behavioral tests.

### Tissue Processing

Mice were deeply anesthetized based on weight with a mixture of 90-150 mg/kg of ketamine and 7.5-16 mg/kg of xylazine and transcardially perfused with 4% paraformaldehyde (PFA) in 1× phosphate buffer saline (PBS) (Thermo fisher Scientific, USA) after an initial flush with 1×PBS. Brains were collected and post-fixed with 4% PFA in 1×PBS overnight and then the tissues were dehydrated in 30% sucrose in 1×PBS. Brains were embedded in optimal cutting temperature compound (O.C.T. Compound, SAKURA, 4583, USA) and then sliced either as coronal or sagittal (50 m) sections on a cryostat microtome (CM 3050 S, Leica biosystems, USA).

### Immunohistochemistry and Image Acquisition:

Myelin quantification and neurofilament staining was performed by Eriochrome Cynanine (Eri-C) stain according to previously published protocols (Sahinkaya et al., 2014).

For Nissl staining, the frozen sections were stained with 0.1% cresyl violet for 15 min, rinsed with PBS, dehydrated with graded alcohol (from 70-100%), followed by xylene treatment and mounted with Permount. For immunofluorescence staining, free floating sections were blocked with 2% Bovine serum albumin (BSA) and 0.1% Triton (Tx)-100 for 1 h at room temperature and then sequentially incubated with primary antibodies overnight at 4° C. and the fluorescent secondary antibodies for 1 h at room temperature. Primary antibodies include: rat anti-proteolipid protein (PLP) (IDDRC hybridoma, courtesy Dr. Judith Grinspan), rabbit anti-myelin basic protein (MBP) (1:250, Abcam, Cat: ab40389), rabbit anti-NG2 (1:250, US biological, Cat: C5067-70D), mouse anti-Olig2 (1:100, Millipore, MABN50), mouse anti-Neuronal Nuclei (NeuN) (1:1000, Millipore, Cat: MAB377); rabbit anti-aspartoacylase (ASPA) (1:1000, Millipore, Cat: GTX110699), rabbit anti-cleaved Caspase (1:200, cell signaling; Cat: #9579); rabbit anti-calbindin (1:250, Swant, Cat: CB38). Secondary antibodies used were: AlexaFluor-488- or AlexaFluor-647-conjugated secondary antibodies against rabbit, mouse or rat (1:1000; Invitrogen). Nuclei were counterstained with DAPI.

### Immunoblotting:

To determine protein levels for key myelin proteins, PLP and MBP levels in cerebellum and forebrain at P14, P21 and end-stage, the respective brain tissues were lysed in RIPA buffer (Thermo Fischer Scientific, USA) in presence of protease and phosphatase inhibitors (Sigma-Aldrich, USA). Samples were boiled in Laemmli buffer and electrophoresed under reducing conditions on SDS-PAGE gels (4-15% mini-PROTEAN pre-cast gels, Biorad, USA). Proteins were transferred onto a nitrocellulose membrane (Trans-blot Turbo transfer system, Biorad, USA) by electro blotting. The membranes were blocked by blocking buffer (1% non-fat milk, Biorad) prepared in 0.05% tween 20 in Tris buffered saline (TBST) and subsequently incubated overnight at 4° C. with primary antibodies against rat anti-PLP (1:1000, IDDRC hybridoma, courtesy Dr. Judith Grinspan) and rabbit anti-MBP (1:2000, Abcam, Cat: ab40389) diluted in blocking buffer. The membranes were washed five times with TBST, incubated in secondary HRP-conjugated goat anti-rabbit (1:5000, Santa Cruz, Catalogue #sc2357) or goat anti-rat antibody (1:5000, ThermoFisher Scientific, Catalogue #31470) for 1 h in blocking buffer, washed in TBST and developed using standard ECL protocols according to manufacturer's instructions (Pierce ECL, ThermoFisher Scientific). Images were scanned and analyzed by Image J software. For normalization with loading control, mouse anti-actin (company, 1:4000) and mouse anti-Vinculin (Company, 1:2000) were used after stripping as per manufacturer's instructions (One-minute Western Blot Stripping buffer, GM Biosciences).

### Electron Microscopy:

Myelination was further assessed by ultrathin sections for structural analysis using electron microscopy (EM). A separate cohort of mice were perfused transcardially with saline followed by 2% PFA and 2% glutaraldehyde in 0.1M PB (PB; pH 7.4) at endstage (˜day 35) (n=3/group) (Lancaster et al., 2018). Each mouse was dissected and their optic nerve, cervical spinal cord and cerebellum (vermis) were isolated. The tissues were post-fixed for 24 hours, rinsed in 0.1M PB, transferred to 2% OsO₄ in 0.1M PB for 1 hour, then processed for embedding in Epon (Lancaster et al., 2018). Semi-thin sections were cut and stained with alkaline toluidine blue and visualized using a light microscope (Lecia DMR) interactive software (Leica Application Suite). Ultra-thin sections (70 nm) were cut, stained with lead citrate and uranyl acetate and imaged using Jeol-1010 transmission electron microscope (TEM). The images from EM sections taken at 100× from optic nerve were assessed using Image J software and the inner and outer axonal area was measured for g-ratio analysis and quantified in 50 axons per animal with n=3 per group as previously published (Lancaster et al., 2018).

### Oligodendrocyte Cultures:

Cell autonomous effects of mutations were assessed by isolating oligodendrocytes from WT, Tubb4a^{D249N} and Tubb4a^{D249N/D249N} mice in culture. Primary oligodendrocyte precursor cells (OPCs) were isolated from cortex between postnatal day P4-P7 using the Miltenyl Anti-O4 microbeads as described in the supplementary methods. O4+ cells were plated at the density of 20,000 OPCs per well of a 24 well plate. The cells were allowed to proliferate for 5-7 days following which they were differentiated in the media without PDGF and bFGF and with Thyroxine T4 (20 μg/ml; Sigma T0397) for another 5 days. The cells were then fixed with 4% PFA and stained using standard immunochemistry protocol. Briefly, the coverslips were washed twice with 1×PBS, permeabilized with 0.2% Tx-100 and then blocked in 10% normal goat serum (NGS) solution for 1 hour. The primary antibodies were prepared in 5% NGS and incubated overnight at 4° C. The primary antibodies used were oligodendrocyte marker rabbit Olig2 (1:800; EMD Millipore AB9610), mature myelin marker Rat PLP (1:1) and rat MBP (1:1) (IDDRC hybridoma, courtesy Dr. Judith Grinspan), washed next day three times in PBS and incubated with the appropriate secondary fluorescent antibodies (1:500; Anti-rat IgG Alexa Fluor 488, Anti-rabbit IgG Alexa Fluor 647). The cells were then mounted using Prolong gold antifade reagent (Thermo Fisher Scientific) and imaged using a Nikon microscope where images were taken at 20× or 40× objective for analysis of cellular counts.

Cortical Neuron culture:

Cell autonomous effects of mutations were assessed by isolating neurons from WT, Tubb4a^{D249N} and Tubb4a^{D249N/D249N} mice in culture. Primary cortical neurons were isolated from E15.5 embryos as described previously (Guedes-Dias et al., 2019). In brief, the cortex was dissected from each embryo and washed with HBSS then 2.5% trypsin was added to each sample and incubated at 37° C. for 7 mins. The trypsin was removed and washed four times with fresh warm HBSS and then resuspended in attachment media (MEM media, 1% sodium pyruvate, 1% horse serum, glucose and sodium chloride). The cells were triturated using a pipette until it is a homogenous single cell solution. The cells were counted and plated on PLL coated MaTeK plates (only in the center) at the density of 150,000 cells/plate and 100,000 cells/well of a 24 well plate. The media was changed after 4 hours to pre-equilibrated maintenance media (Neurobasal media, 1% glutamax, 1% penicillin/streptomycin, glucose, sodium chloride and 2% B27 solution). After 3 days, 20-30% of the media was removed and fresh media with the mitotic inhibitor AraC was supplemented to then neurons. The neurons plated in the 24 well plate was assessed for cell survival analysis, axonal and dendritic length measurements. Neurons were stained with MAP2 (1:200) and TuJ1 (1:200) to label dendrites and cell body/axons respectively and imaged at 20× or 40× objective for cellular counts and measuring axonal and dendritic length. Axonal and dendritic length was measured using the Neurite tracer plugin in FiJi software.

### Live-Imaging of EB3 Dynamics

Microtubule dynamics was assessed by live cell imaging of EB3-mCherry, where end-binding protein 3 (EB3) tags the growing plus end of the microtubules. On day in vitro (DIV) 6, cortical neurons were transfected with EB3-mCherry using Lipofectamine 2000 (Invitrogen). 20-24 h after transfection, maintenance media was exchanged to low fluorescence Hibernate E imaging media (BrainBits) supplemented with 2% B27 and 2 mM GlutaMAX. The neurons were imaged in an environmental chamber at 37° C. on a PerkinElmer UltraView Vox Spinning Disk Confocal system with a Nikon Eclipse Ti inverted microscope using a Plan Apochromat 60×1.40 NA oil immersion objective. Images were acquired with a Hamamatsu EMCCD C9100-50 camera driven by Volocity software (PerkinElmer) at a framerate of 2 seconds per frame for 600 seconds. Quantification of EB3 dynamics was performed as previously described (Guedes-Dias et al., 2019). The ImageJ macro toolset KymoClear was used to generate kymographs (Mangeol et al., 2016). The KymoClear toolset passes a Fourier filter on the original kymograph allowing for automated discrimination of anterograde, retrograde or static components and improves the signal-to-noise ratio of EB3 comets without affecting quantitative analysis of the data. The tracks of individual EB3 comets were manually traced using a custom MATLAB GUI (Kymograph Suite) and used to determine run-length, run-time and velocity of each comet. The investigator was blinded for the neuronal genotype during both image acquisition and kymograph analysis.

### Statistical Analysis

All graph data are presented as the mean±standard error mean (SEM). In text ‘n’ represents the number of mice used per experiment unless indicated otherwise. Gait abnormalities, righting reflex, rota-rod, and weight assessments were analyzed by two-way ANOVA with repeated measures followed by the post-hoc Tukey test. For Grip strength and ambulation, one-way ANOVA with post-hoc Tukey test was performed. Survival was analyzed by the Kaplan-Meier method, and the differences between groups were estimated by the Gehan-Breslow-Wilcoxon test. Comparisons in myelin quantification, NeuN, ASPA, NG2, Olig2 and cleaved caspase 3 counts and fluorescent intensity were analyzed by ordinary two-way ANOVA with multiple comparisons post-hoc Tukey tests. Neuronal survival, axon and dendritic length and assessment of OL markers examined in vitro were compared using one-way ANOVA with the post-hoc Tukey test. The EB3 dynamics in neurons was analyzed using one-way ANOVA or two-way ANOVA with repeated measures. All statistical analyses were performed using Prism 7.0 (GraphPad Software) with p<0.05 considered statistically significant.

### Mouse Genotyping:

DNA extraction from tail was carried out by using HotSHOT method as published previously (Truett et al., 2000). The taq-takara system was used to amplify the 541 bp of PCR product, using the forward primer 5′CCGAGAGGAGTTTCCAGACAGACAGGATC3′(SEQ ID NO: 3) and the reverse primer 5′GCTCTGCACACTTAACATCTGCTCG 3′ (SEQ ID NO: 4). The products of amplification were subjected to sequencing to identify the genotype of mouse.

### Behavioral Tests:

Ambulatory angle: Gait abnormalities were determined by measuring the ambulatory/hind limb foot angle. Ambulatory angle was performed with some modifications (Feather-Schussler and Ferguson, 2016). The ambulatory angle was measured weekly at P7, P14, P21, P28 and P35. Three measurements were performed using data only when the pup was performing a complete walk in a straight line with both feet flat on the ground.

Ambulation: Ambulatory deficits were detected as described previously with some modifications (Feather-Schussler and Ferguson, 2016). Ambulatory behavior was assessed at P7, 10 and 14. Based on these strategies of crawling and walking, we examined if transgenic mice attain their crawling/walking skills later than their WT littermates. Mice were scored using a single trial on crawling, gait symmetry and limb-paw movement during a straight walk (FIG. 1 and Table 3).

TABLE 3
Ambulation Scores

| Ambulatory skills | Score |
|---|---|
| No response | 0 |
| Asymmetric crawling | 1 |
| Symmetric crawling | 2 |
| Walking | 3 |

As illustrated in FIG. 1E, throughout crawling, the whole hind paw touches the ground as designated by (#) and tail is low or touching the ground. When transitioning from crawling to walking head begins to rise. Walking is seen only when the toes of the hind paw touch the ground and the heel is elevated, designated by [##] (Feather-Schussler and Ferguson, 2016). Symmetric limb movement was described as hind paws meet front paws during each step, and each step smoothly transitions to the next step. A mouse exhibiting asymmetric limb movement had inconsistent paw placement and transitions from one step to the next are not smooth.

Hanging Grip strength: Grasping abilities of front and hind paws were determined by performing hanging grip strength. Hanging grip strength was performed as described in Feather-Schussler and Ferguson, 2016, supra. The trials were repeated three times and average angle was calculated. A 13″×9.5″ metal screen wire mesh was used to perform the hanging grip strength on P14. The protractor was placed in parallel to wire mesh so as to measure the angle at which pups fall. The mouse was placed on the screen and was allowed to adjust to this novel environment for ˜10 sec. The screen was slowly inverted to 180 degree and approximate angle of the screen when pup falls off was recorded. The trials were repeated three times and average angle was calculated.

Righting reflex: Righting reflex tests the mice trunk control and motor co-ordination. Righting reflex was carried out as described in Feather-Schussler and Ferguson, 2016, supra. The righting reflex test was performed at every week from P7, P14, P21, P28, P35 and then daily when Tubb4a^{D249N/D249N} mice demonstrated motor impairment. Three trials were carried out and a total of 1 min was given for each trial, if needed. The righting reflex test was performed at every week from P7, 14, 21, 28, 35 and then daily when Tubb4a^{D249N/D249N} mice demonstrated motor impairment. The mouse was placed on its back on the bench pad and was held in position for 5 sec. Mouse was released and the time it takes to return to flat position was noted. Three trials were carried out and a total of 1 min was given for each trial, if needed.

Rota-rod: Motor coordination, strength and balance were assessed using a Rota-rod (UGO BASILE S.R.L, Gemonio, Italy). The latency to fall from the rotating rod for three test trials after a training period (P21) was recorded in each trial and mean was used for analysis. To evaluate progressive motor loss, the Rota-rod test was performed on P28 and P35, and the mean of latency of fall between the age of mice were used for statistical analysis. To adapt with the apparatus, on day 1, mice were placed on the cylindrical rod rotating with the constant speed of 5 rpm for 100 sec. The next day, mice were placed at an accelerating speed from 5 to 30 rpm for 300 seconds over three trials/day with an inter-trial interval of approx. 20 minutes. On day 3, three test trials were performed at an accelerating speed of 5-30 rpm for 300 sec.

### Immunohistochemistry, Image Analysis and Quantification

### Myelin Quantification and Neurofilament Staining:

Free-floating sections were treated with 10% hydrogen peroxide in methanol for 20 min and blocked for 1 h with the blocking buffer (4% bovine serum albumin (BSA) in 1×PBS with 0.1% Triton-x (Tx)-100) followed by 1:500 chicken anti-NF (1:500, Aves, cat: NFH) in the same blocking buffer for overnight at 4° C. Following primary antibody incubation, sections were incubated with biotinylated anti-chicken secondary antibody (1:1000, Aves, cat: B-1005) for 1 h and developed by Elite Avidin Biotin Conjugate (Vector) and visualized with DAB substrate. Slides were rinsed in tap water, treated with acetone, rinsed in tap water and immersed in eriochrome cyanine (Eri-C) solution for 30 min. The sections were differentiated in 5% Iron Alum, rinsed with tap water and differentiated in borax ferricyanide. Sections were dehydrated, cleared, mounted with permount (Fischer Scientific, USA) and coverslipped. For myelin quantification in corpus callosum and cerebellum (3-4 sections per mouse, n=at least 3 for PND 14, P21 and end-stage), images were captured in bright field mode in Keyence BZ-X-700 digital microscope. Images captured at 10× magnification were tiled with the Keyence BZ-X software. The stained area was measured with the Image J software and then related to the total white matter.

NeuN And Caspase count: For striatal and cerebellar sections (3-4 sections per mouse, n=3-4 for P14, P21 and end-stage), images were captured at 20× and 63X, respectively, using z-optical sections at 1-2 μm intervals by using Leica DM6000B fluorescence microscope. Image J software was used to count NeuN+ cells with DAPI. Analysis was performed blindly and counts are reported as profiles/mm².

ASPA and Olig2/NG2 counts: The protocol was followed as publish previously with some modifications (Lee et al., 1985). Sections labeled for ASPA and NG2+/Olig2+ and counterstained with DAPI were used to quantify the total number of ASPA and NG2+/Olig2+ cells in corpus callosum. All images were captured at 40× oil immersion lens on an Olympus laser scanning confocal microscope by using z-stack with 0.5-1 μm optical intervals. Image J software was used and a standardized sample box (0.01 mm²) was placed in the regions of interest. Positively labeled cells were identified as ASPA+ or NG2+/Olig2+ or Olig2+ cells which were superimposed with the DAPI nuclei. The final counts are reported as profiles/mm².

Fluorescent density and area quantification: To quantify fluorescent positive area and density, the images were captured at 10× magnification using Leica DM6000B fluorescence microscope. Image J software was used, the areas of interest was selected and Integrated area density and grey values were calculated. Following formula was used to calculate fluorescence.

Corrected total fluorescence=Integrated Density−(Area of selected cell×Mean fluorescence of background readings).

### Oligodendrocyte Isolation:

In brief, cortex was microdissected and isolated from each mouse brain and meninges was removed to avoid contaminating cultures. The cortex was cut into smaller pieces and dissociated using the Neural Dissociation kit (Miltenyl Biotec (P), 130-092-628) and as per the protocol, each sample was incubated with enzyme mix 1 (Enzyme P and Buffer X) for 15 mins at 37° C. Next, enzyme mix 2 was added and the tissue was mechanically dissociated using a fire-polished Pasteur pipette and incubated for 10 mins at 37° C. This process was repeated two more times to obtain a single cell solution which was applied to a 70 μm strainer and centrifuged for 10 mins at 300×g. The cell pellet was resuspended in 90 μl of PBS buffer (pH 7.2) containing 0.5% bovine serum albumin. 10 μl of Anti-O4 Microbeads was added to the cell pellet, mixed and incubated for 15 mins in the refrigerator. The cells were then washed with 1-2 ml of buffer and centrifuged at 300×g for 10 mins. The supernatant was aspirated, and the cells were resuspended in 500 μl of buffer. The MS MACS column was placed in the magnetic field was rinsed with 500 μl of buffer and then the cell suspension was applied to the magnetic column. The flow-through containing unlabeled cells was collected and the column was rinsed three times with 500 μl of buffer. The column is then removed from the separator and placed on a suitable collection tube, where the appropriate amount of media is applied and immediately flushed by pushing the plunger into the column. This fraction is the O4+ cells was suspended in media containing neurobasal media with 2% B27, 1% penicillin streptomycin, 1% Glutamine and the growth factors human bFGF (100 μg/ml; R&D 233-FB/CF), human PDGF-AA (100 μg/ml; Peprotech 100-13A) and human NT3 (100 μg/ml; Peprotech 450-03).

### Antisense Oligonucleotide Synthesis

Eleven ASOs were synthesized by Integrated DNA Technologies. We performed in vitro screening of these ASOs to identify the best ASO design. Mouse Oli-neu cells were electroporated with 1 μM, 5 μM and 10 μM of ASO concentrations at 150 V in 100 μL media with 100,000 cells/well on the NEPA21 electroporation system (NEPA GENE, USA). Following electroporation, cells were transferred to a poly-L-ornithine coated plate and placed in an incubator. Forty-eight hours post-treatment, cells were washed with PBS and then RNA extraction was performed using PureLink™ RNA Mini Kit (ThermoFisher Scientific, Cat: 12183018A) according to manufacturer's instructions. After treatment with DNAase (Invitrogen), 200 ng of RNA was used for cDNA with SuperScript™ IV First-Strand Synthesis System (ThermoFisher Scientific, Cat: 18091200). The mRNA expression levels of Tubb4a, and an endogenous housekeeping gene encoding Splicing factor, arginine/serine-rich 9 (sfrs9) as a reference, were quantified using real-time PCR analysis (Tagman chemistry) on an Applied Biosystems Quanta Flex 7 (ThermoFisher Scientific, USA). The results were analyzed using the ΔΔCT method.

The following examples are provided to illustrate certain embodiments of the invention. They are not intended to limit the invention in any way.

### Example I

### Mouse Model of H-ABC Disease

In the present example, we describe generation of a knock-in mouse with Tubb4a^{D249N/D249N} mutations as a model of H-ABC, which recapitulates features of the human disease including dystonia, loss of motor function and gait abnormalities. The histopathological features of the mouse model includes both loss of neurons in striatum and cerebellum along with hypomyelination in the brain and spinal cord as observed in patient tissues (Curiel et al., 2017b). We have also explored the functional consequence of mutant tubulin on microtubule polymerization and the cell-autonomous role of Tubb4a mutation in neurons and oligodendrocytes using the Tubb4a^{D249N/D249N} mice. This study provides a promising first model for H-ABC, using the most frequently occurring mutation, key in understanding mechanisms underlying this devastating disease and developing therapies.

### Generation of Tubb4a^{D249N} CRISPR Knock-In Mice

To understand the molecular mechanisms and disease course of classical H-ABC with p.Asp249Asn (p.745G>A) mutation, a knock-in mouse model Tubb4a^{D249N} was generated using CRISPR. Further, these Tubb4a^{D249N} mice were bred to obtain homozygous Tubb4a^{D249N/D249N} mouse colonies (FIG. 1A). Homozygous mice were studied in parallel with Tubb4a^{D249}N mice, similar to previously required homozygous expression for early expression of phenotypes in rodent models of Tubb4a mutations (13) despite heterozygous mutations in affected individuals with H-ABC.

### Tubb4a^{D249N/D249N} Mice Demonstrate Early Disease Onset and Decreased Survival

To determine phenotypes of Tubb4a^{D249N}ad Tubb4a^{D249N/D249N} mice, mice were examined on a daily basis after birth. From birth to post-natal day (P) 8, WT, Tubb4a^{D249N} and Tubb4a^{D249N/D249N} mice appear similar with respect to their development. However, by ˜P9, Tubb4a^{D249N/D249N} mice begin to display tremulous behavior. This phenotype progressively worsens with age and mice become severely ataxic and dystonic over time. By ˜P35-P40 the mice are incapable of feeding themselves, show slow righting ability and this time-point is described as their “end-stage” (compassionate end-point) (p<0.001, FIGS. 1B and 1C). Additionally, weight measurements indicate that Tubb4a^{D249N/D249N} mice start to show gradual weight reduction from P35 with significant reduction at ˜P37 (15.02±0.67) compared to Tubb4a^{D249N}(18.57±0.38) and WT (17.44±0.43) mice (p<0.001, FIG. 1L). Tubb4a^{D249N} mice appear normal and do not show any evident behavioral phenotype.

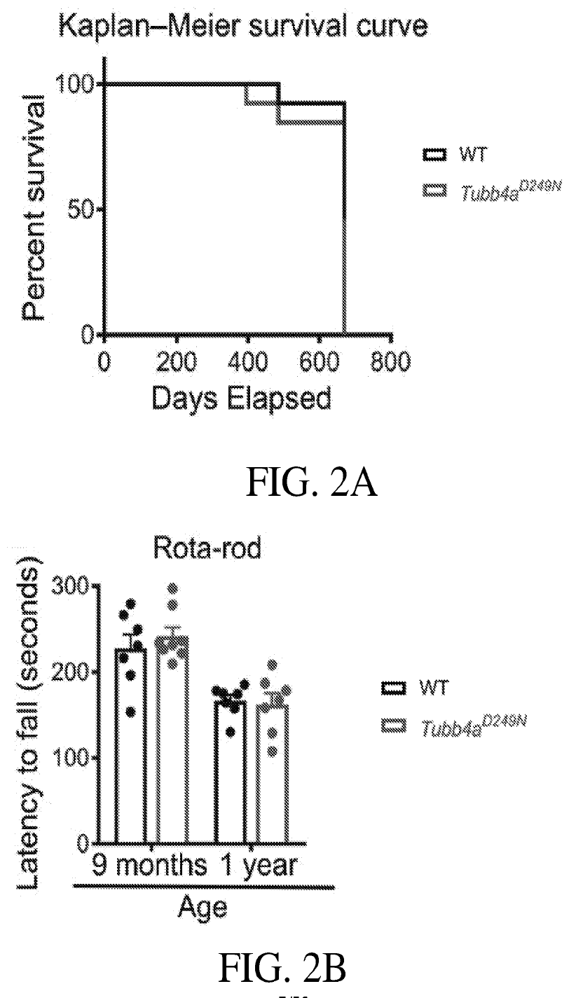

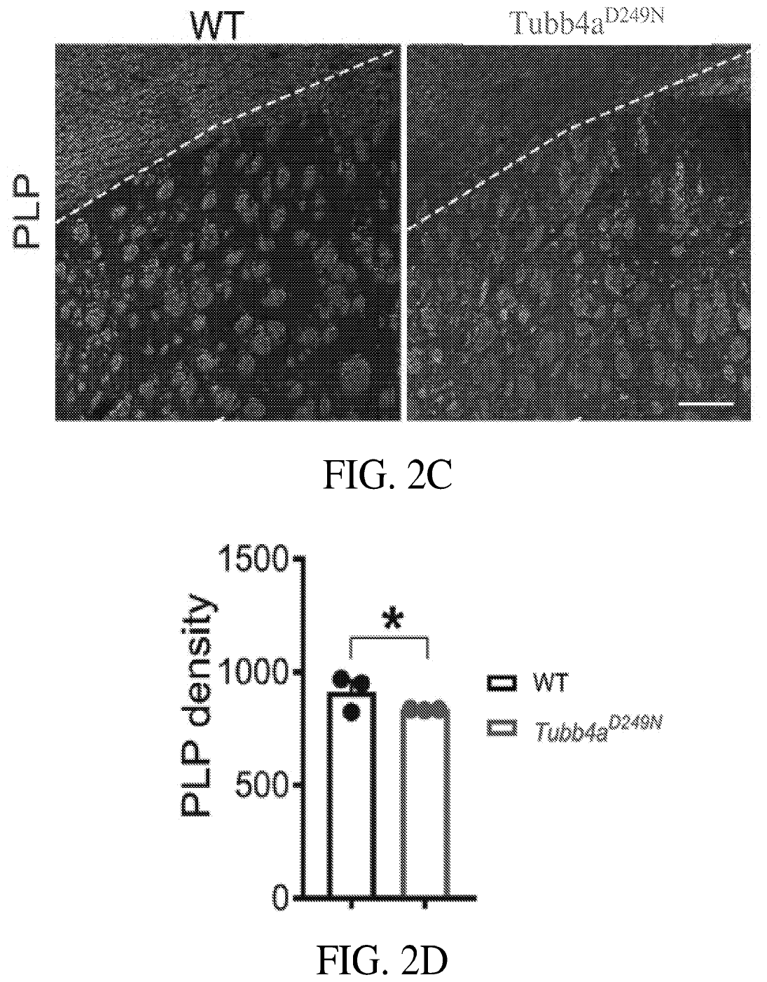

Tubb4a^{D249N} mice show similar survival compared to their WT littermates and die mainly because of their advanced age (Kaplan-Meier survival curve, FIG. 2A).

### Tubb4a^{D249N/D249N} Mice Exhibit Gait Abnormalities

Given that the individuals affected by H-ABC display delayed ambulation, ataxia, gait abnormalities and progressive motor dysfunction, we decided to perform comprehensive behavioral analysis on Tubb4a^{D249N} and Tubb4a^{D249N/D249N} mice to see if mice similarly recapitulate H-ABC behavioral phenotypes (FIG. 1D) (1, 10, 20).

To investigate if Tubb4a^{D249N} and Tubb4a^{D249N/D249N} mice display abnormal gait, ambulatory angle or hind limb foot angle was determined. Starting at P14, Tubb4a^{D249N} mice walk without deficits in ambulatory angles while Tubb4a^{D249N/D249N} mice display significantly wider ambulatory angle compared to their WT littermates (p<0.001, FIGS. 1G and 1H; P14—71.82±4.26 vs 43.16±2.46, P21—84.00±7.56 vs 61.96±2.93 and P35—81.03±5.37 vs 52.66±1.87). The wide ambulatory angle of Tubb4a^{D249N/D249N} mice is consistent with the gait instability seen at these ages, as the pups and adults need to increase the angle of their rear paws to stabilize their gait and to support their balance and coordination (14).

Ambulation was assessed at earlier developmental time points, when mice are transitioning from crawling to walking, in WT, Tubb4a^{D249N} and Tubb4a^{D249N/D249N} mice (See Table 3 for ambulation score). At P7, Tubb4a^{D249N} and Tubb4a^{D249N/D249N} mice show similar asymmetric crawling as that of WT controls (FIG. 1E). By P10, Tubb4a^{D249N/D249N} mice still exhibit asymmetric limb movement with crawling gait, as seen in younger pups, and additionally display tremors relative to that of WT littermate controls (P10—1.20±0.13 vs 2.20±0.25) (p<0.05, FIGS. 1E and 1F) while Tubb4a^{D249N} have more symmetric limb movement with a crawling/walking gait. All mice including Tubb4a^{D249N/D249N} mice achieve walking skills by P14, however, homozygous Tubb4a^{D249N/D249N} mice still continue to display tremors.

### Tubb4a^{D249N/D249N} Mice Exhibit Progressive Motor Dysfunction

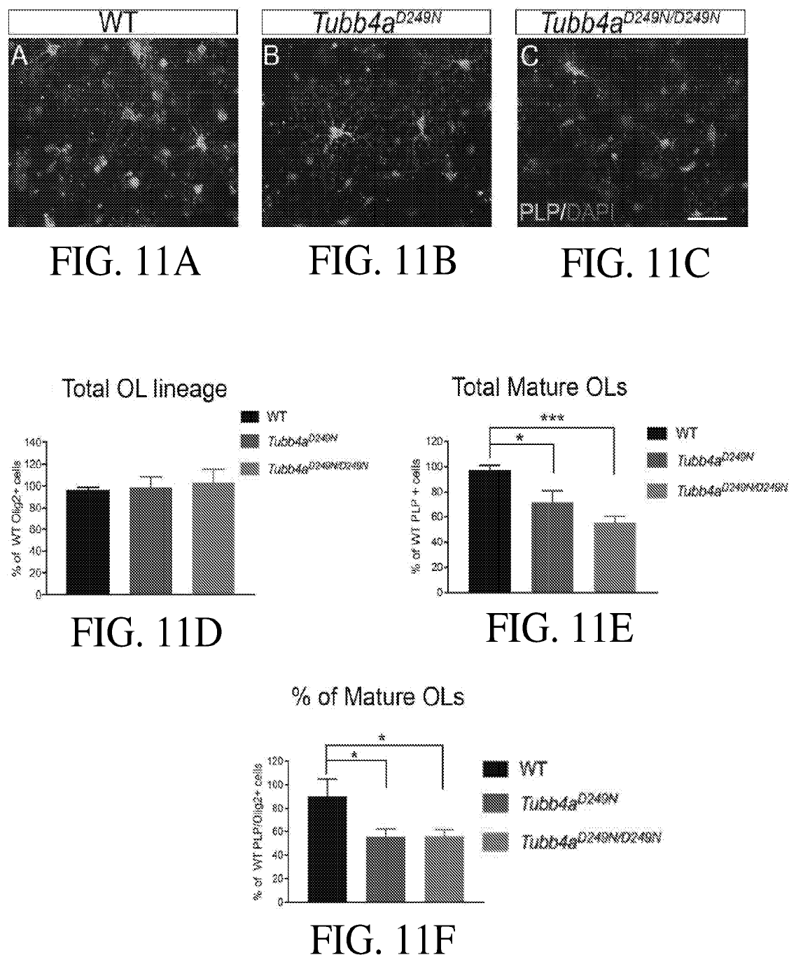

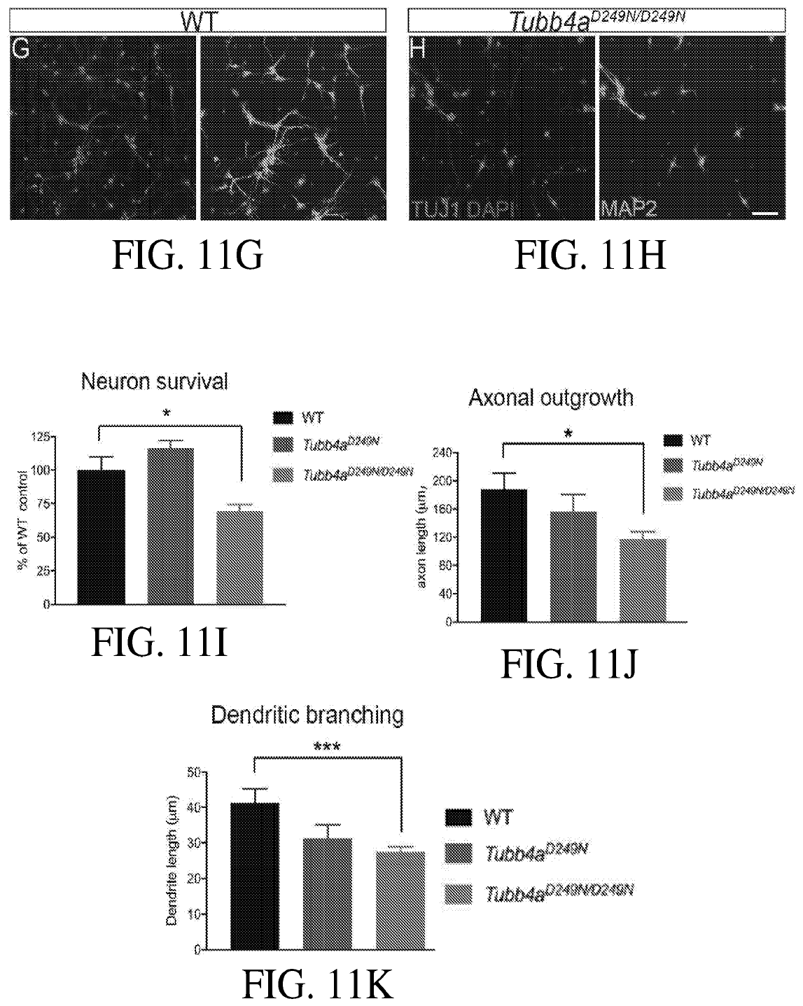

To assess if Tubb4a^{D249N} and Tubb4a^{D249N/D249N} mice show additional deficits in their motor development, their grasping ability was determined by performing hanging grip strength at P14. Grasping with all four paws is essential for mice to climb and run through uneven surfaces (14) and poor performance in the grip strength indicates that mice have impaired motor ability. Tubb4a^{D249N} mice show similar grip strength compared to their WT littermates, while Tubb4a^{D249N/D249N} mice fall at significantly lesser angles than WT (86.40±2.51 vs 103.9±1.84; p<0.001, FIGS. 11 and 1J).

To evaluate if disease is progressive and whether mutations in Tubb4a in juvenile mice affects co-ordination and balance, we assessed performance of these mice on the rota-rod at P21, P28 and P35. There are no significant differences between the performance (measured by latency to fall in seconds) of heterozygous Tubb4a^{D249N} mice compared to WT littermates, while homozygous Tubb4a^{D249N/D249N} mice show shorter duration of latency to fall on an accelerating rota-rod at P21 (107.1±7.58 vs 213.8±15.16 secs), P28 (101.0±10.30 vs 239.0±10.76 secs) and P35 (20.69±6.71 vs 257.9±11.40 secs) with progressive worsening over time (p<0.001, FIG. 1K).

To test if Tubb4a^{D249N} mice develop behavioral deficits at later stages, rota-rod testing was performed at ages of 9 months and 1 year and did not show any changes relative to WT mice (FIG. 2B).

Finally, righting reflex was assessed every week from P7 to P35, followed by daily assessment from P35 to the end-stage of Tubb4a^{D249N/D249N} mice. The surface righting ability tests mouse trunk control and co-ordination ability (14). Additionally, the ability to right themselves is necessary for self-care and feeding and is used as an ethical end-point in this study (21, 22). By P14, all mice are able to right themselves immediately, however, after day 38, Tubb4a^{D249N/D249N} mice show significant impaired righting abilities compared to their WTs littermates (p<0.001, FIG. 1L).

Tubb4a^{D249N/D249N} Mice Exhibit Severe Developmental Delay in Myelination and Both Tubb4a^{D249N} and Tubb4a^{D249N/D249N} Mice Ultimately Show Loss of Myelination

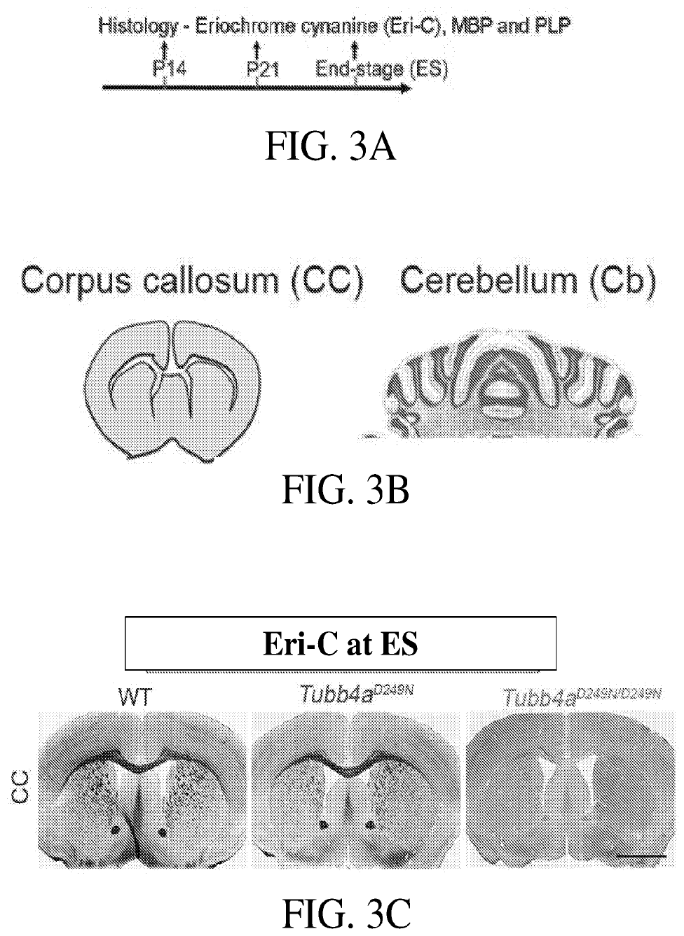

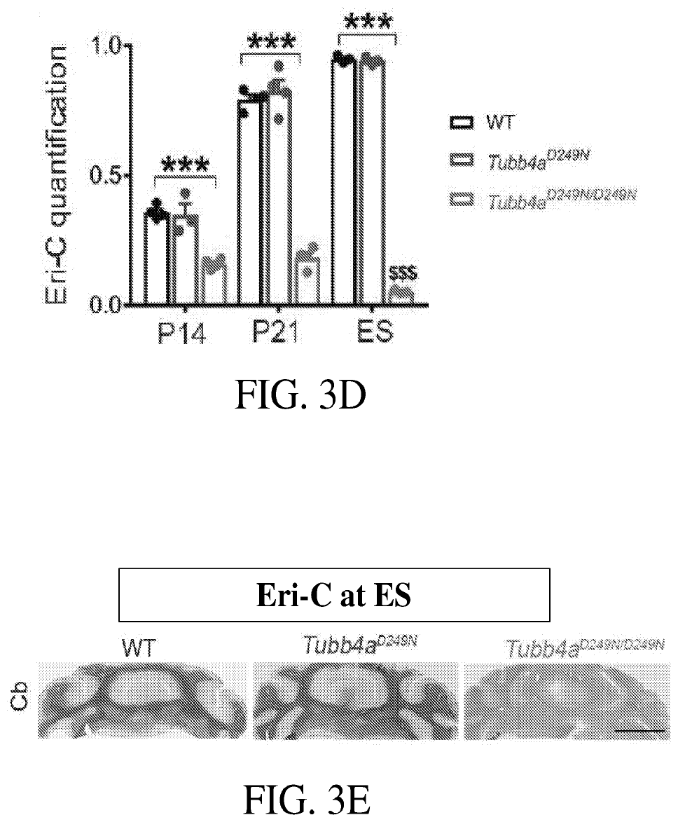

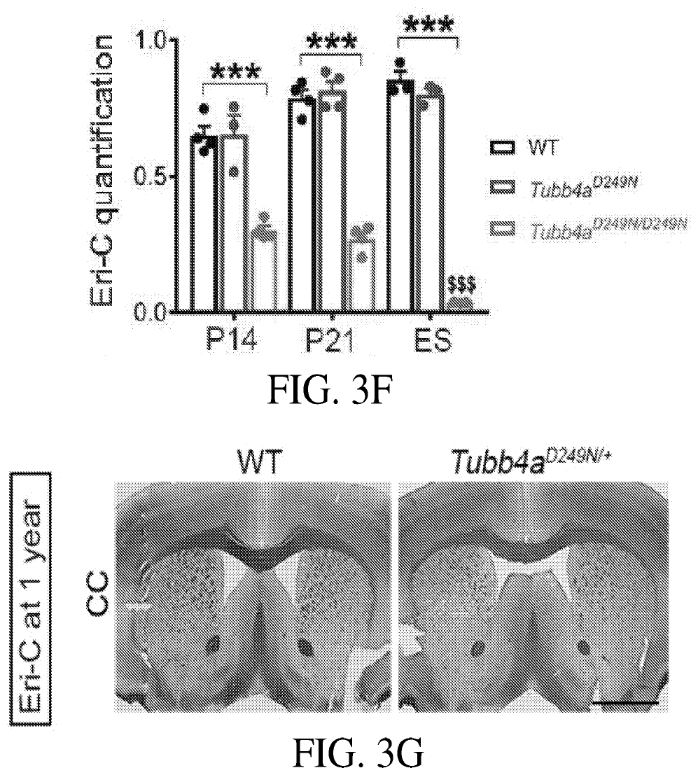

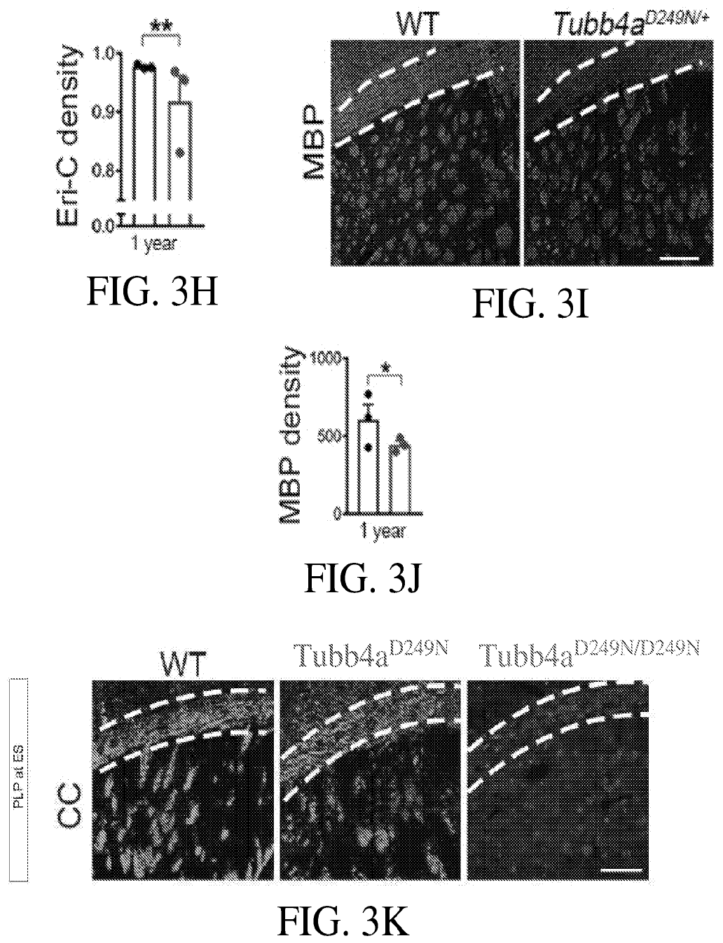

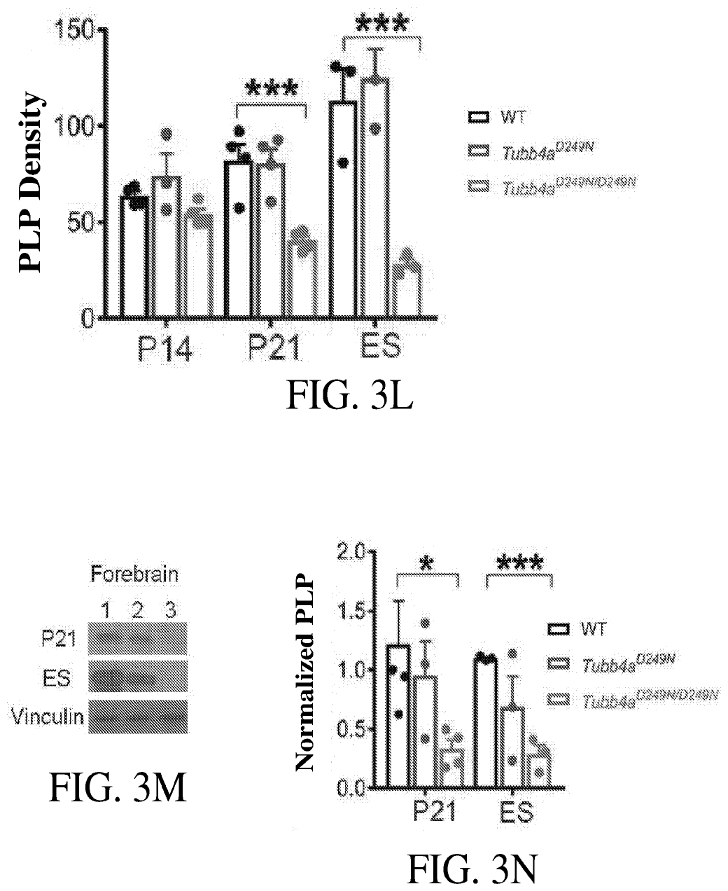

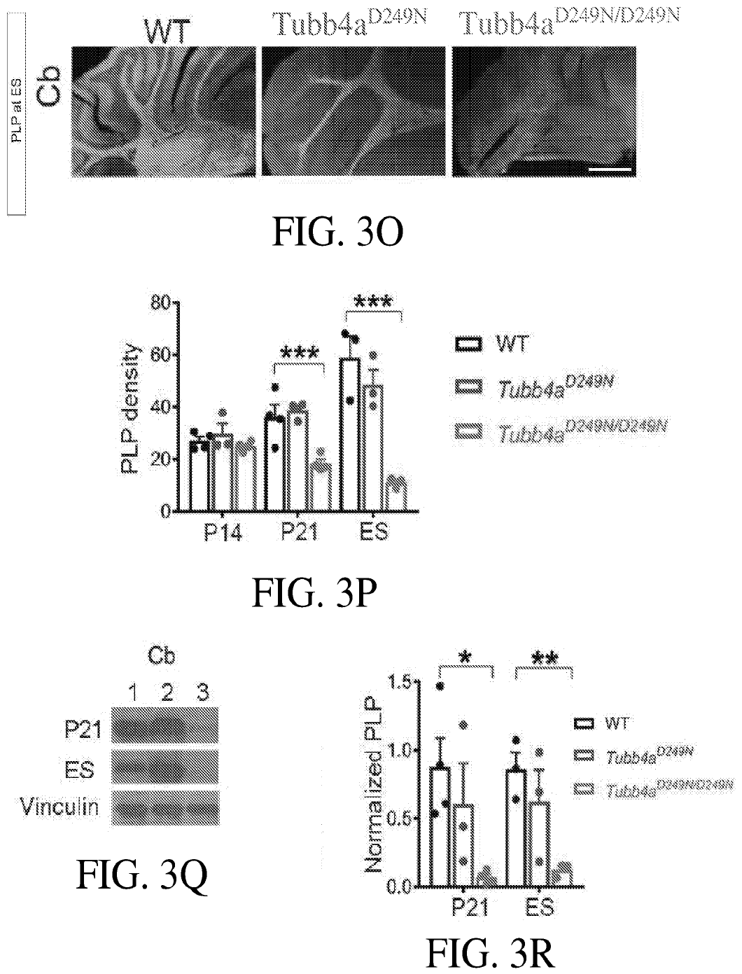

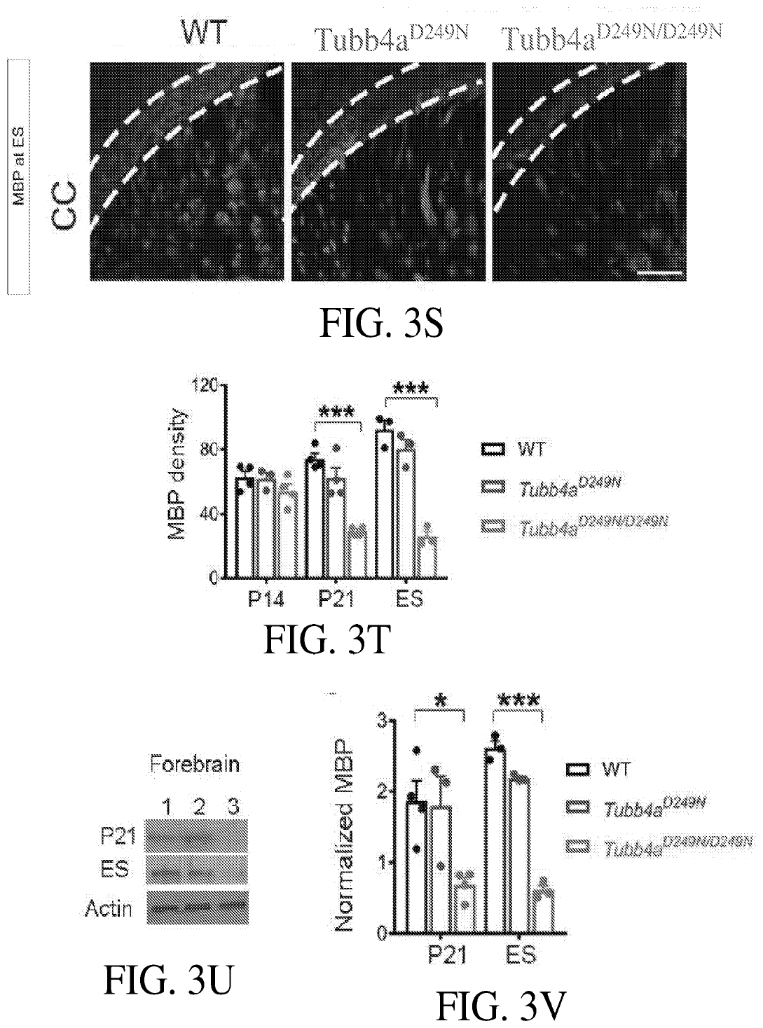

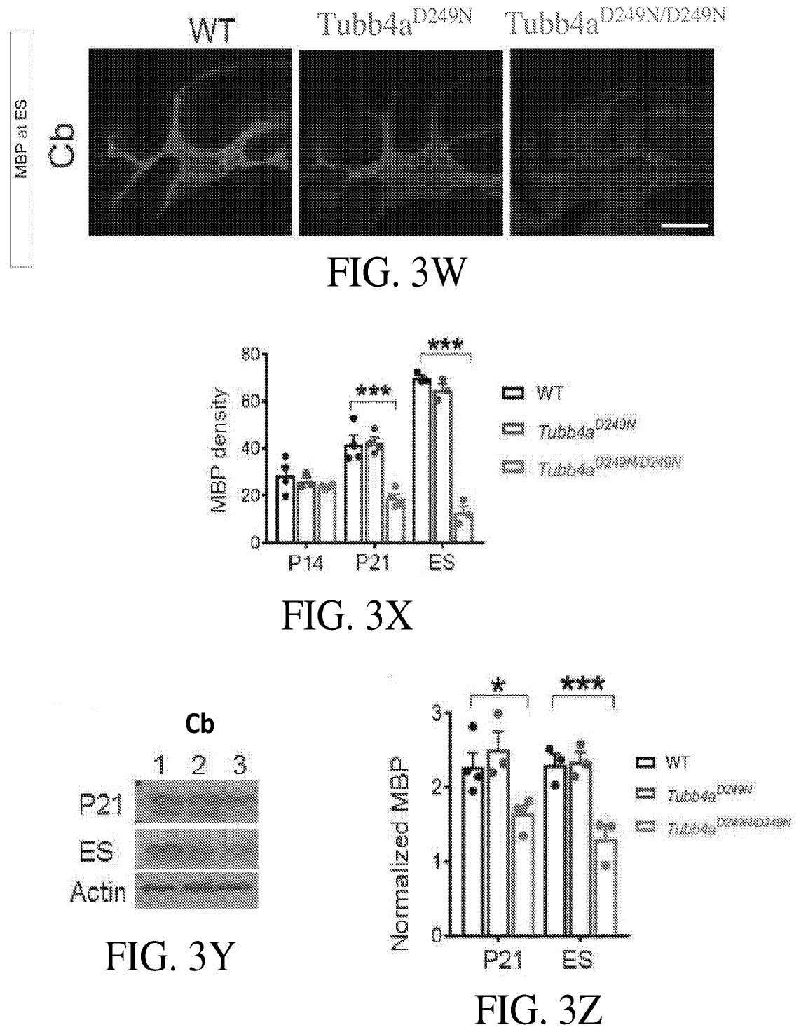

Tubb4a^{D249N} and Tubb4a^{D249N/D249N} mice recapitulate the myelin abnormalities typically seen in individuals affected by H-ABC (3, 10) and Tubb4a^{D249N/D249N} mice show developmental or degenerative loss of myelin. Myelination in mice starts at birth in the spinal cord and has a nearly adult pattern by P21 (23). Tubb4a^{D249N/D249N} mice display severe lack of typical myelin development as measured by immunohistochemistry (Eri-C) in corpus callosum and cerebellum at P14 and P21 relative to WT littermates. Tubb4a^{D249N/D249N} mice show an initial delay in myelination (P14—p<0.001, P21—p<0.001, FIG. 6B) with loss of previously achieved myelination by the end-stage (corpus callosum—0.053±0.004 vs 0.948±0.009 and for cerebellum—0.037±0.001 vs 0.854±0.033) (p<0.001, FIG. 3C-3F). To assess later onset of myelin phenotypes in Tubb4a^{D249N} mice, Eri-C staining was conducted at 1 year of age, and shows decreased myelin staining (p<0.001, FIGS. 3G and 3H).

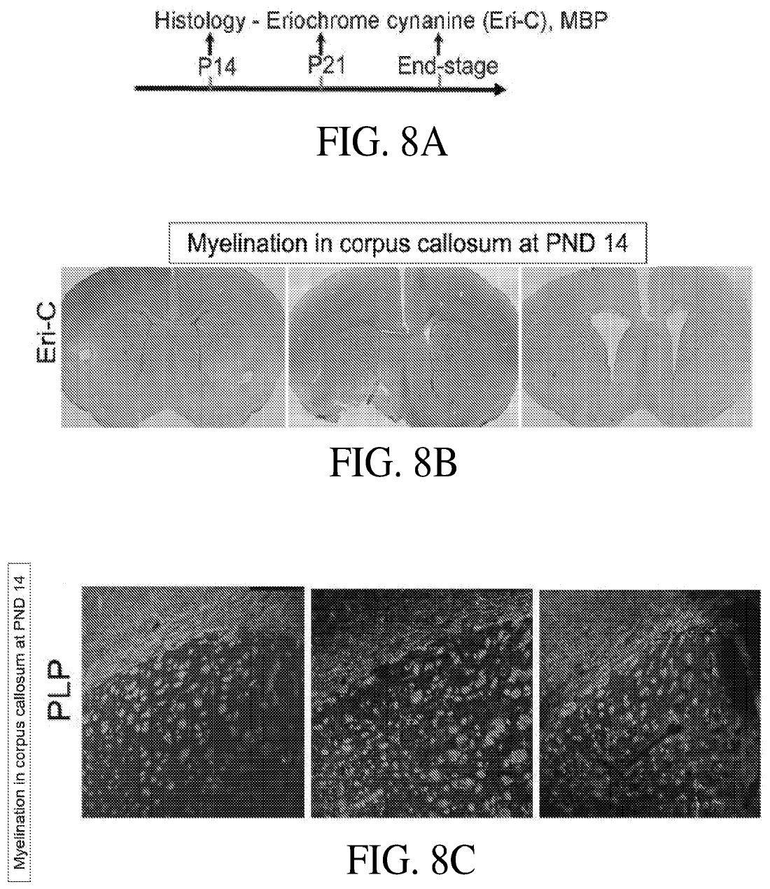

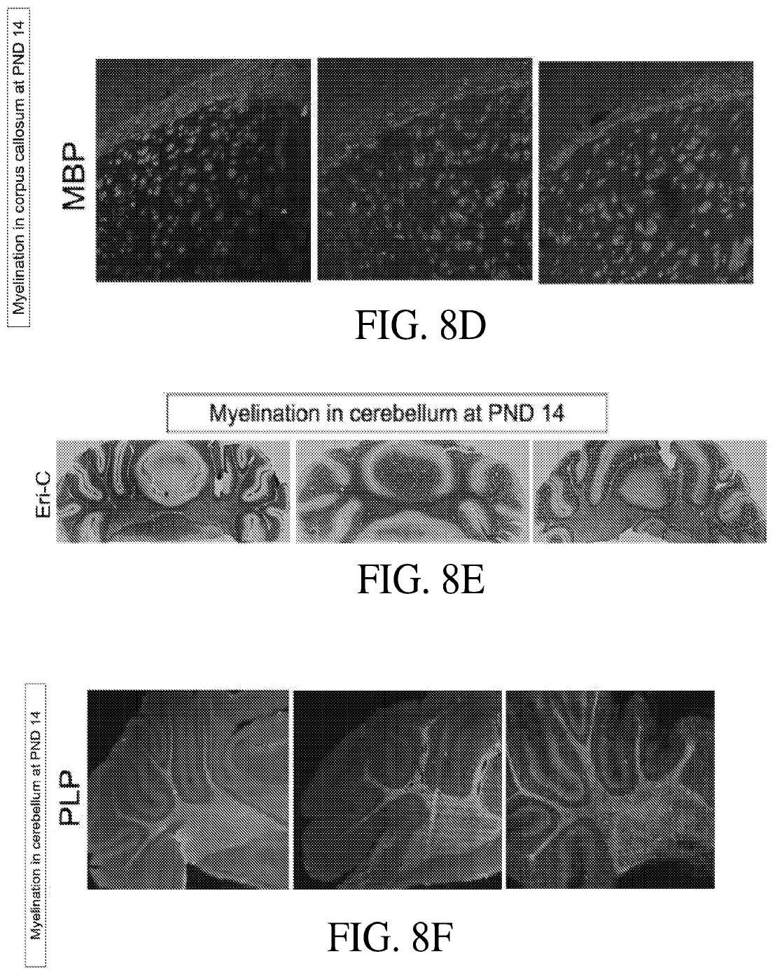

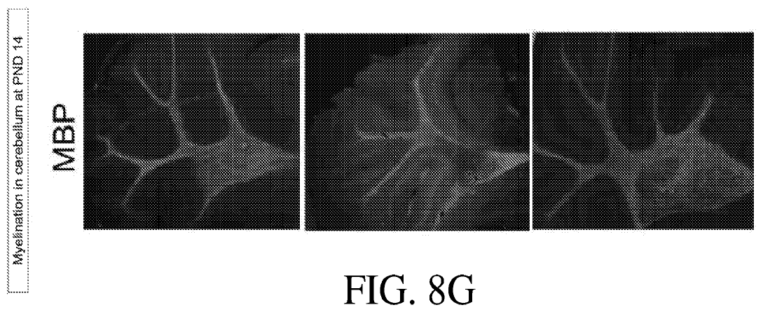

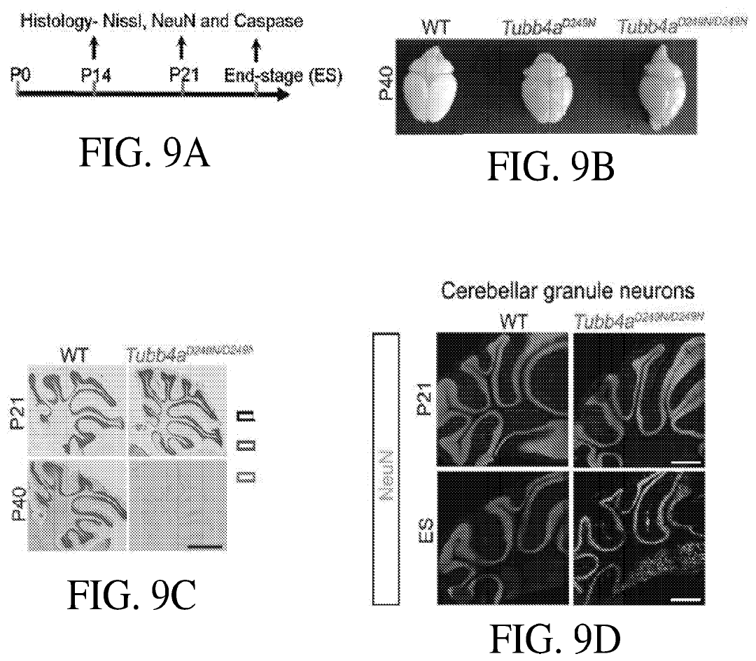

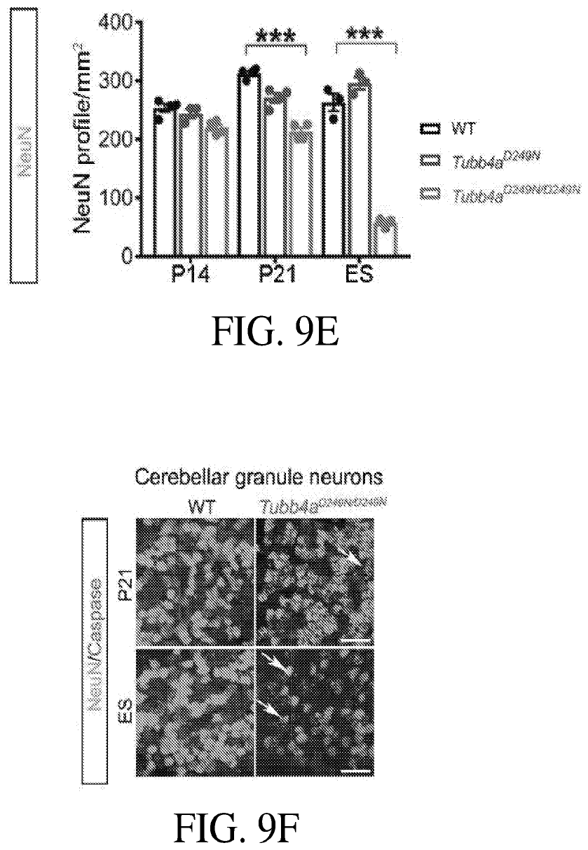

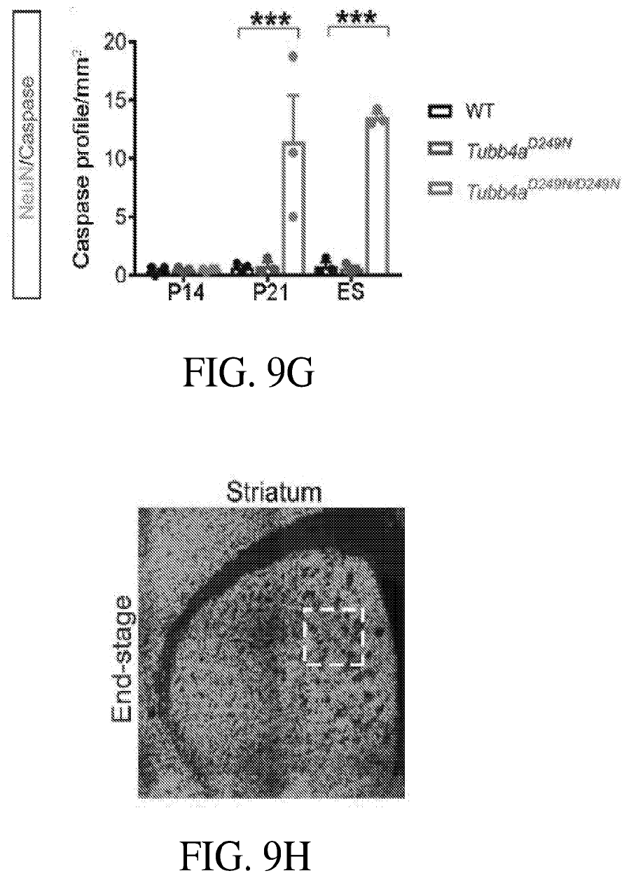

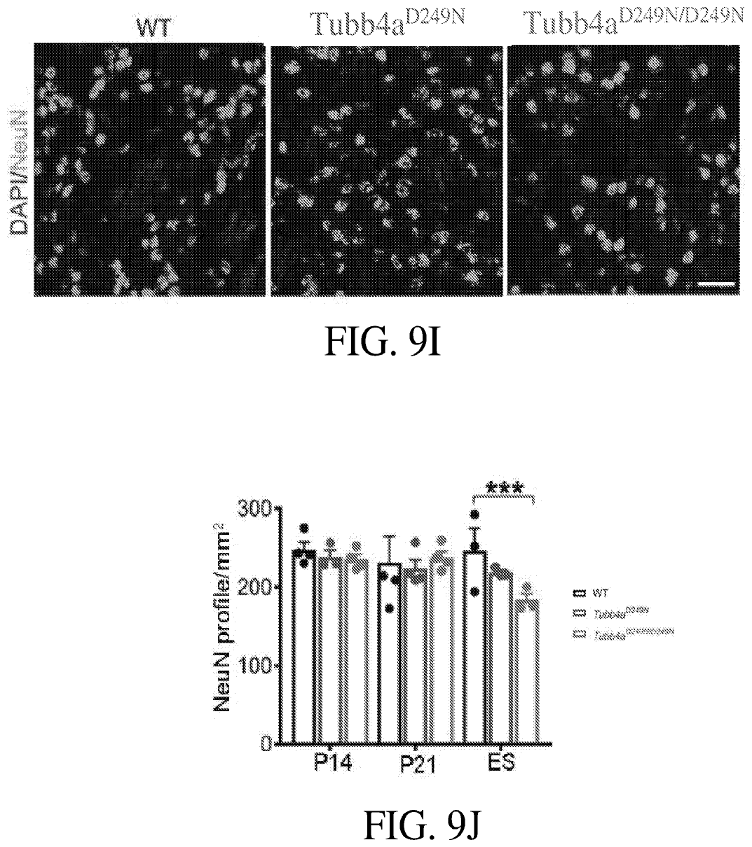

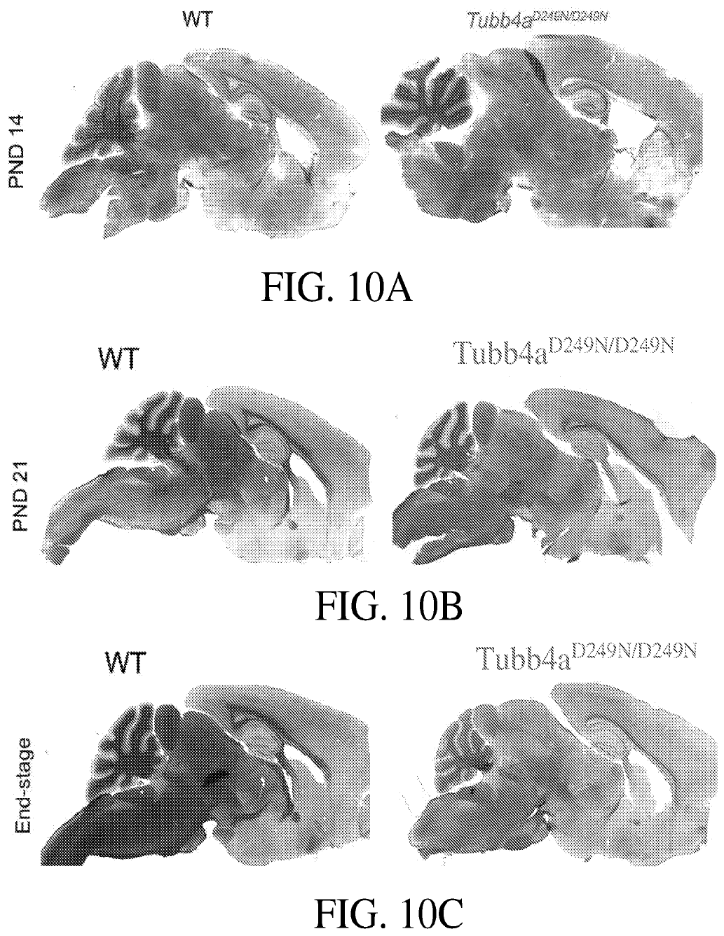

Additionally, Tubb4a^{D249N/D249N} mice show absence of major myelin proteins by immunostaining. In Tubb4a^{D249N/D249N} mice, MBP and PLP expression is unchanged at P14 in corpus callosum and cerebellum (FIG. 6C-6G), but significantly diminished by P21 (p<0.001, FIG. 8C-8G) and end-stage in corpus callosum (p<0.001, FIG. 3K-3L and FIG. 3S-3T; PLP—27.99±3.02 vs 113.46±16.18 and MBP—25.73±3.42 vs 92.40±5.76) and cerebellum (p<0.001, FIG. 3O-3P and FIG. 3W-3X; PLP—11.11±1.17 vs 58.90±8.19 and MBP—12.65±2.98 vs 69.79±1.20). Tubb4a^{D249N} mice show comparable MBP and PLP expression in corpus callosum and cerebellum as that of WT's at P21 and end-stage of Tubb4a^{D249N/D249N} mice. However, by 1 year, Tubb4a^{D249N} mice display diminished levels of PLP (p<0.05, FIG. 2C-2D) and MBP (p<0.05, FIG. 3I-3J) as compared to WTs.

Similar decreases of PLP and MBP levels in Tubb4a^{D249N/D249N} mice are detected using Western blot in forebrain (PLP—0.286±0.08 vs 1.101±0.01, FIG. 3M-3N and MBP—0.605±0.06 vs 2.615±0.09, FIG. 3U-3V) and cerebellum (PLP—0.123±0.03 vs 0.860±0.12, FIG. 3Q-3R and MBP—1.307±0.178 vs 2.306±0.14, FIG. 3Y-3Z) at P21 (p<0.05) and end stage (p<0.001).

### Tubb4a^{D249N/D249N} Mice Show Severe Loss of Oligodendrocytes

In view of both the developmental and degenerative abnormalities of myelin formation in Tubb4a^{D249N} and Tubb4a^{D249N/D249N}, we assessed both oligodendrocytes (OLs) and oligodendrocyte precursor cells (OPC's) counts (23). OLs were examined in corpus callosum at P14, P21 and end-stage by immunostaining counts using ASPA, a marker of OLs.

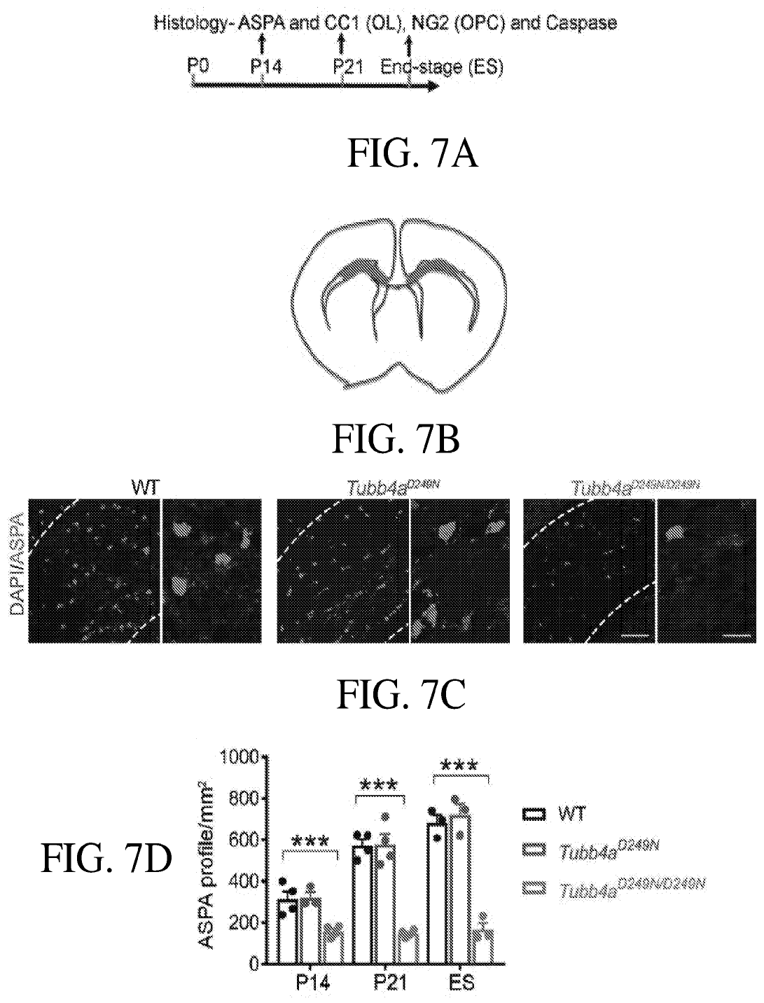

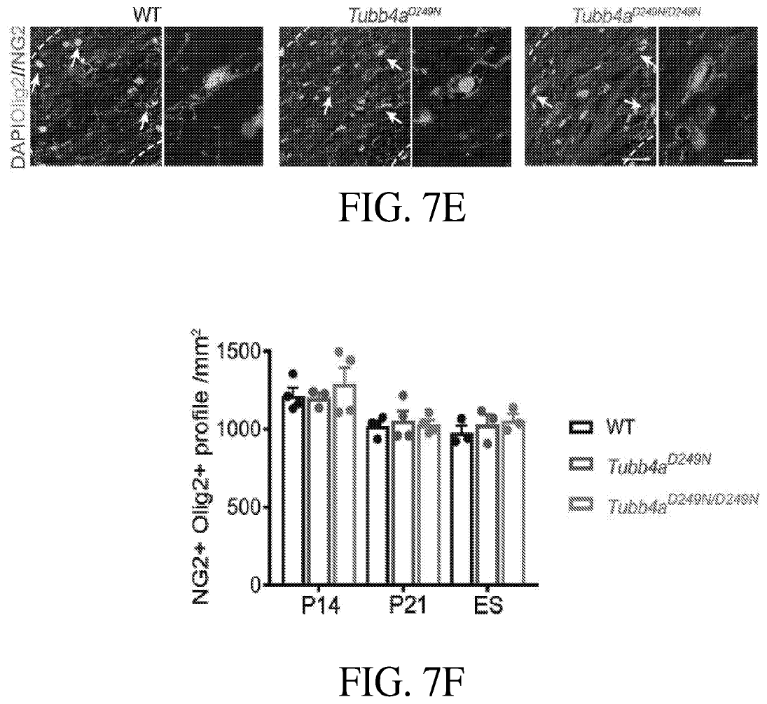

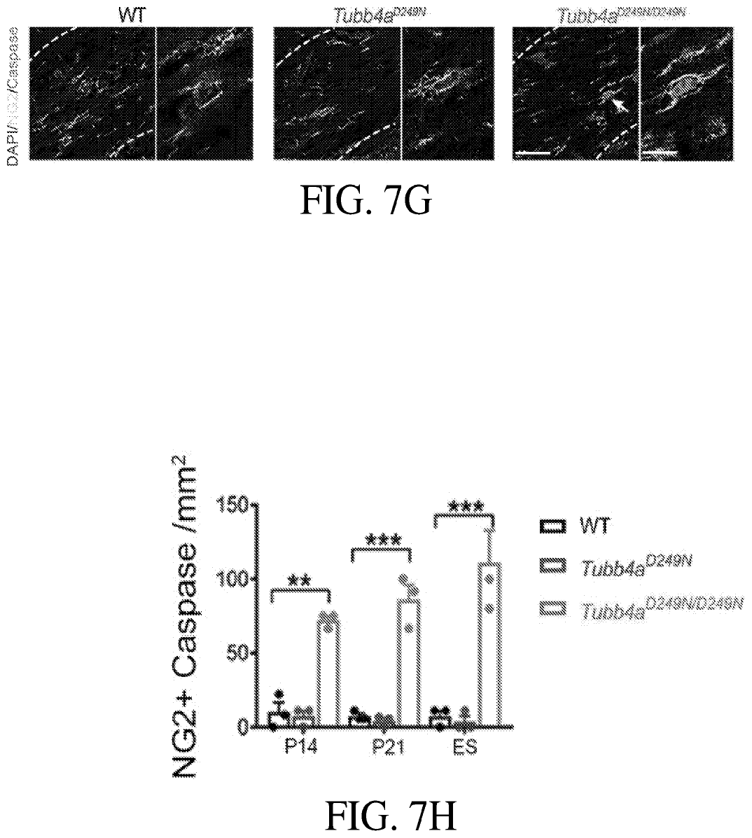

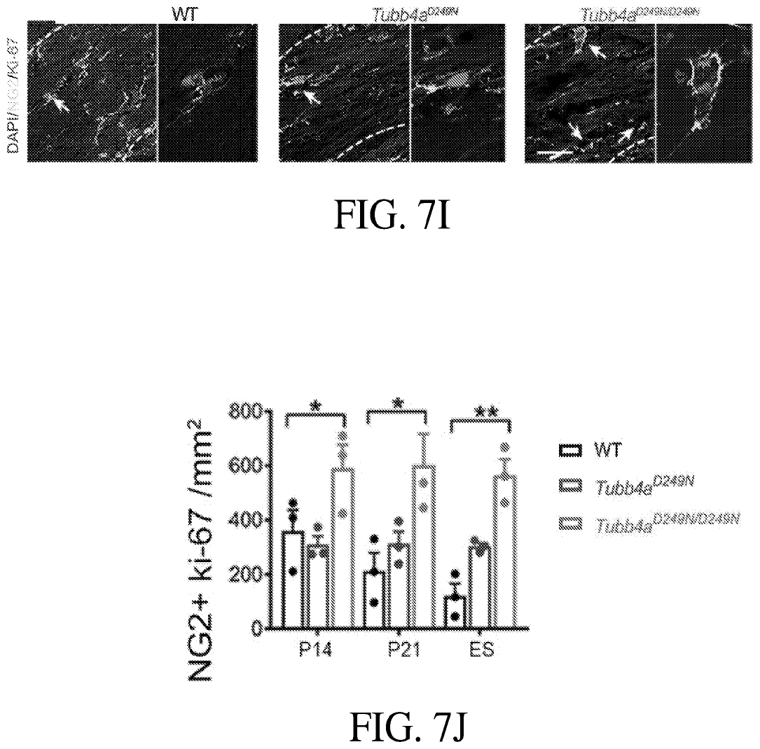

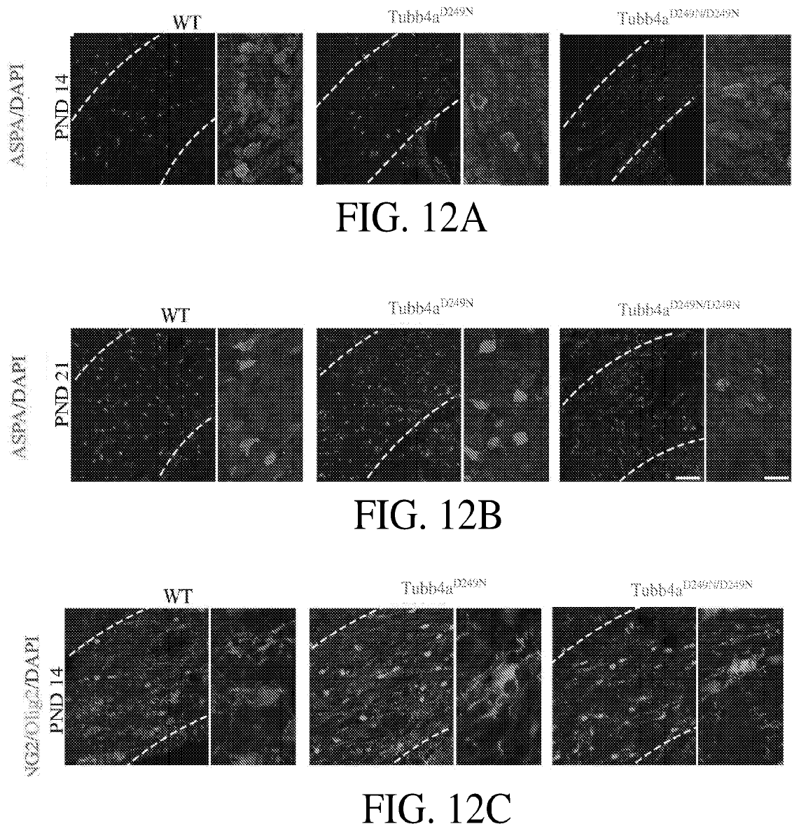

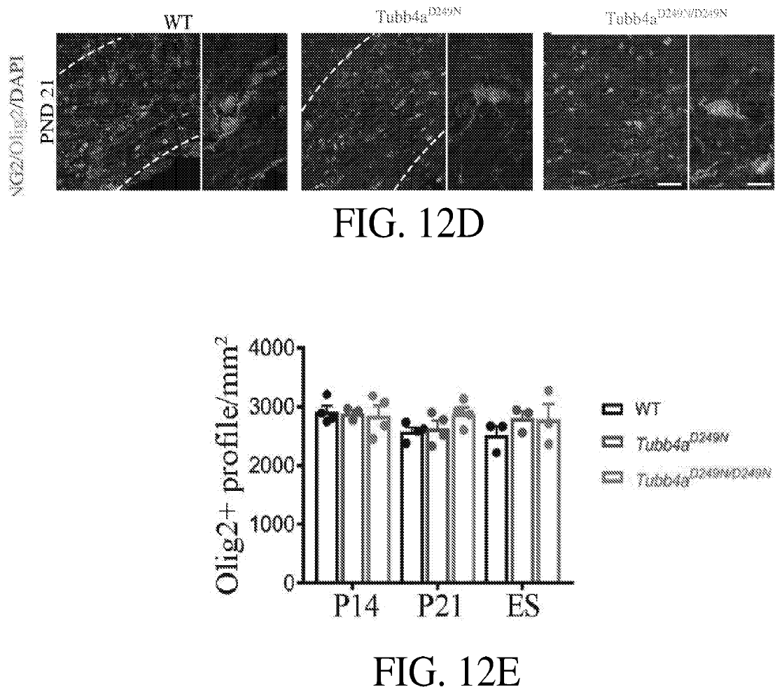

At P14, P21 and end-stage (FIG. 7C-D), number of ASPA positive OLs are greatly reduced in Tubb4a^{D249N/D249N} mice as compared to their WT littermates in corpus callosum (p<0.001, 166±32.34 vs 681±38.45). To evaluate if there is change in number of OPC's, we counted double positive NG2+ Olig2+(a pan OL lineage marker) cells in the corpus callosum. NG2+ Olig2+ counts are unchanged in Tubb4a^{D249N/D249N} mice at P14, P21 (FIG. 12A-B) and end-stage (FIG. 7E-F) Additionally, Olig2+ cell counts are comparable in Tubb4a^{D249N/D249N} mice at P14, P21 and end-stage time-points (FIG. 12C-E) suggesting no change in number of all OL lineage cells.

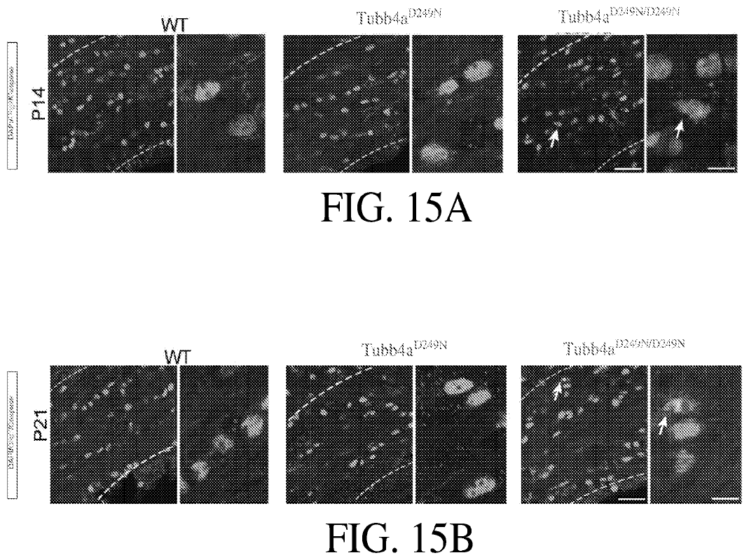

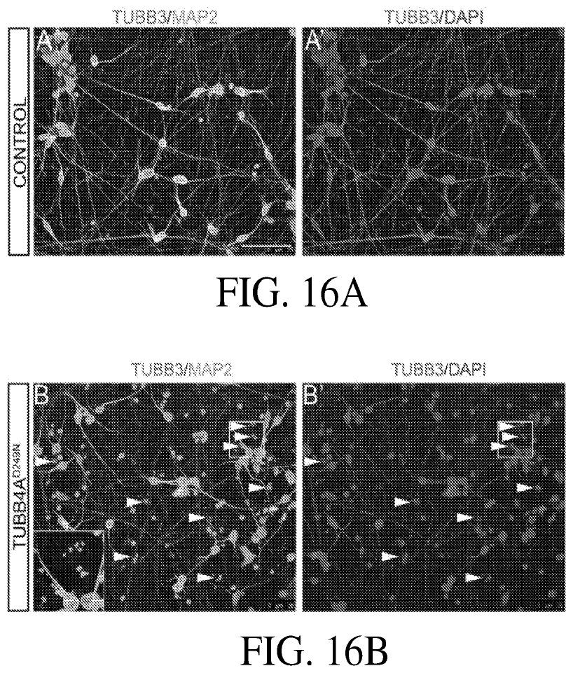

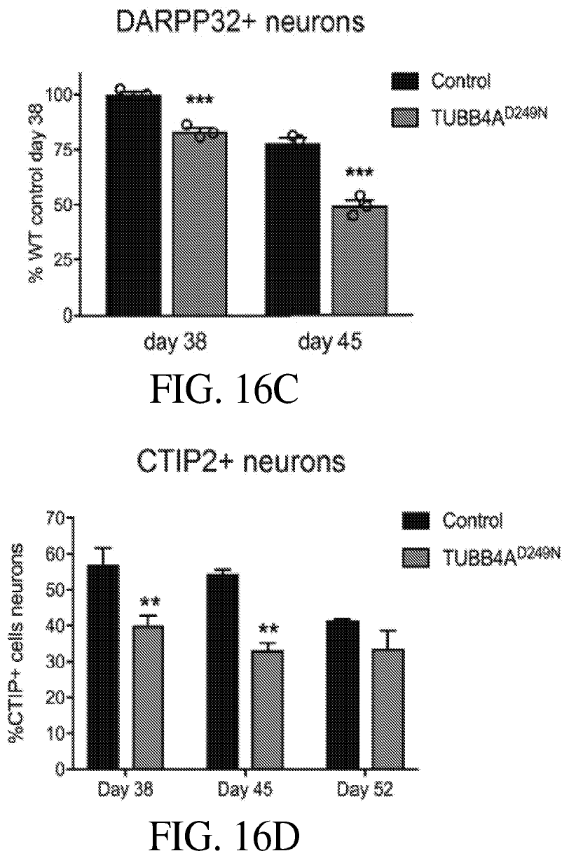

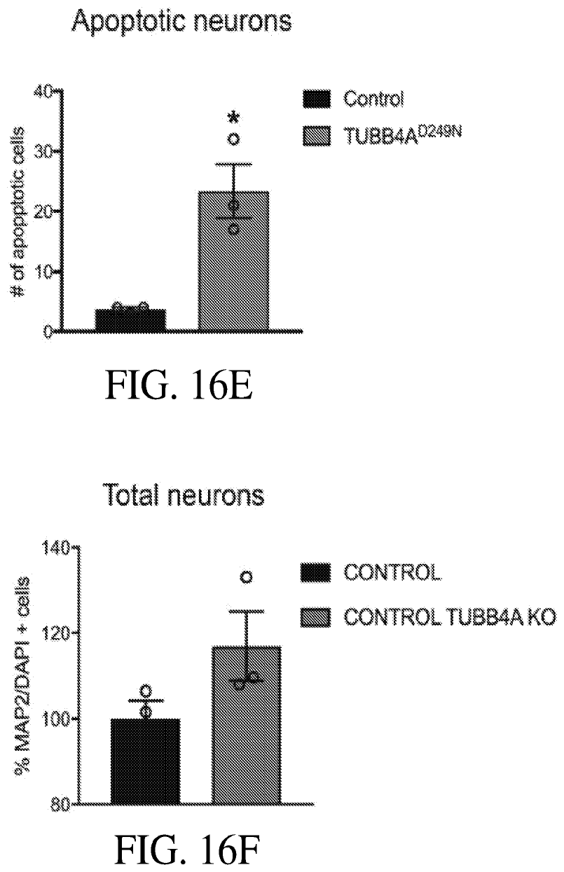

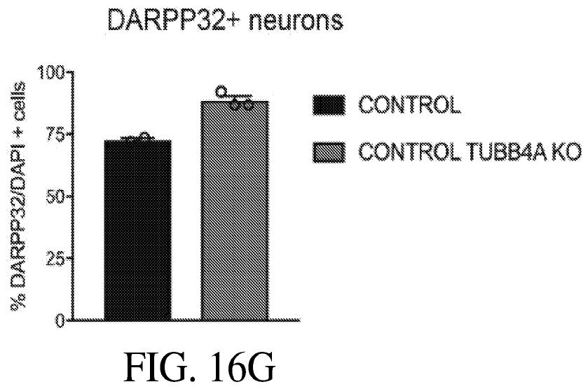

To examine if OLs lineage cells are undergoing cell apoptosis, double immunostaining was performed with caspase, a cell apoptotic marker and Olig2, an OL lineage marker. We found the significant and progressive increase in number of double positive caspase and OL (ASPA) cells in the corpus callosum in Tubb4A^{D249N/D249N} mice at P14, P21 (FIGS. 15A-D) and end-stage (p<0.001, FIG. 7G-H). As OPC's and Olig2 cell counts are spared in Tubb4a^{D249N/D249N} mice, we can speculate that the mutation in Tubb4a most likely causes toxic effects on mature OL resulting in loss of mature OL.

Ultrastructure Analysis Corroborates Myelination Deficits in Tubb4a^{D249N} and Tubb4a^{D249N/D249N} Mice

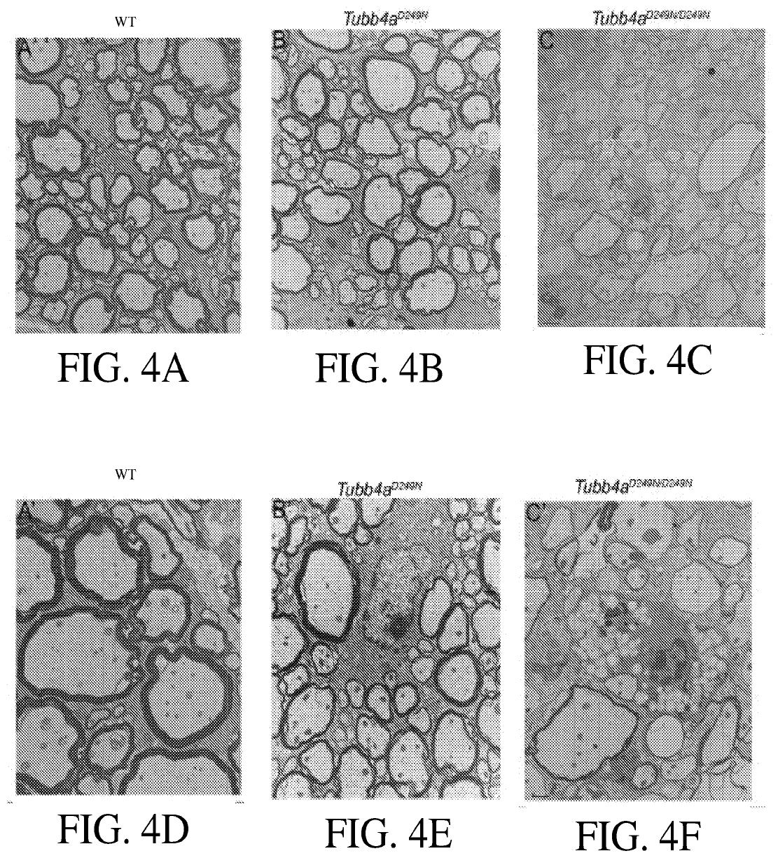

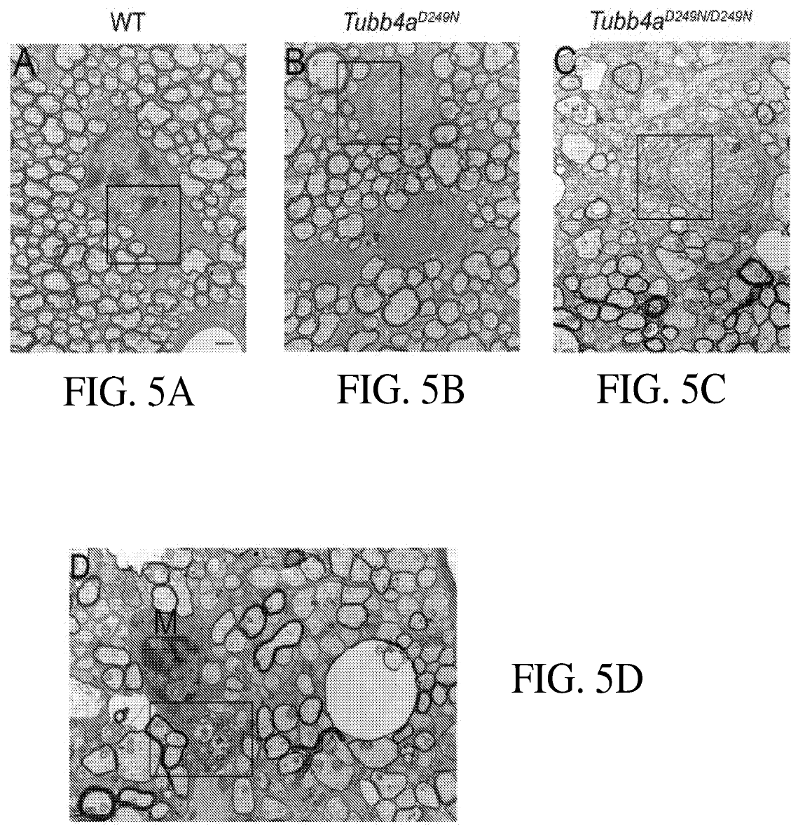

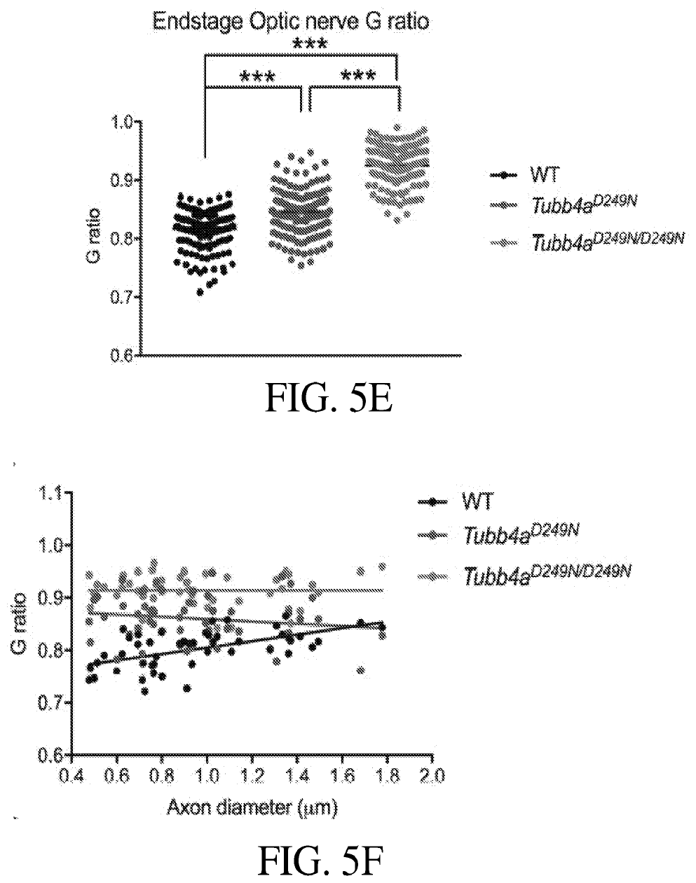

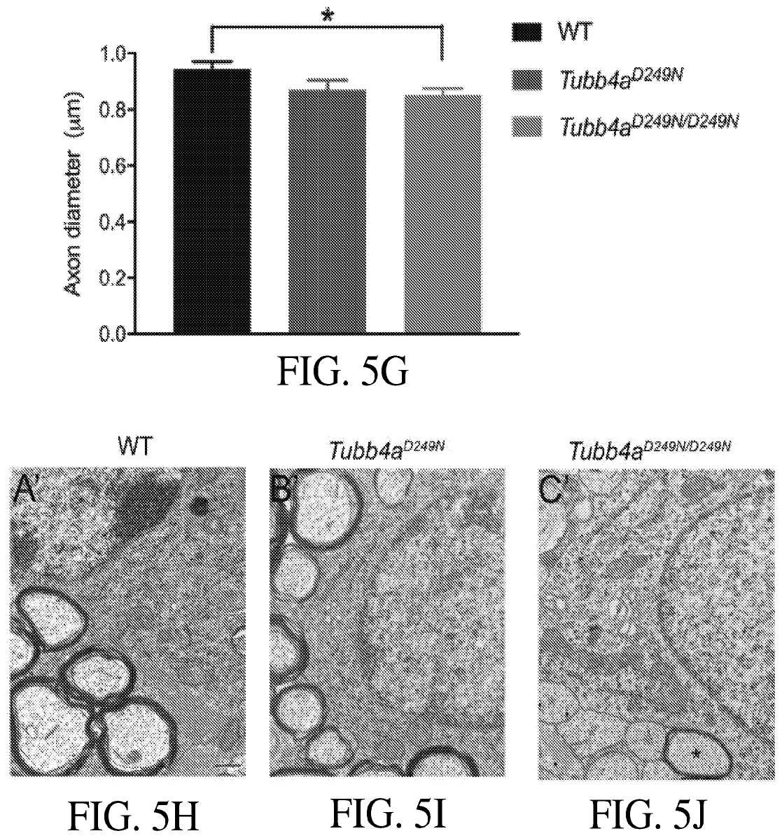

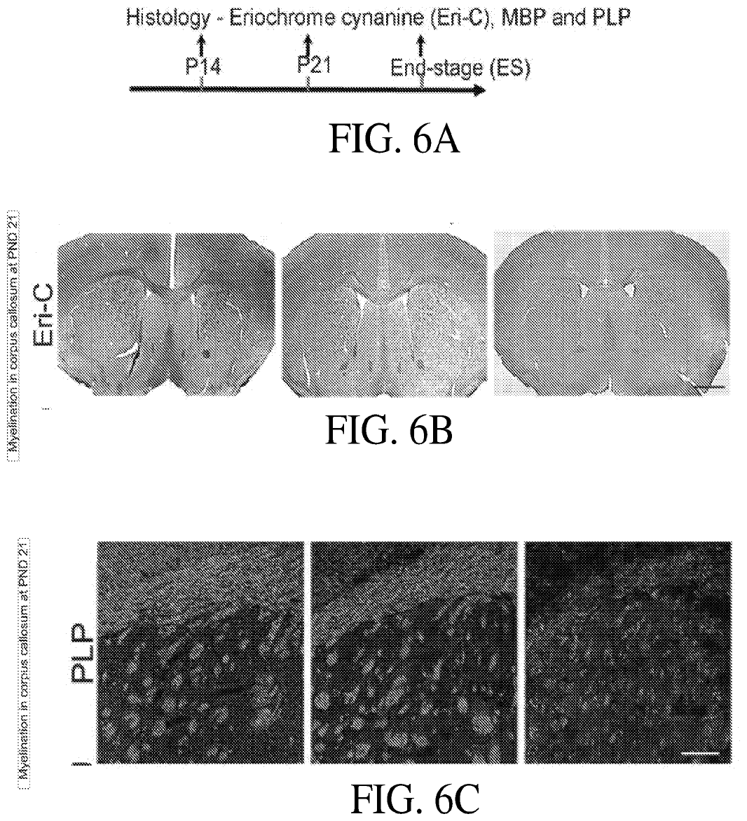

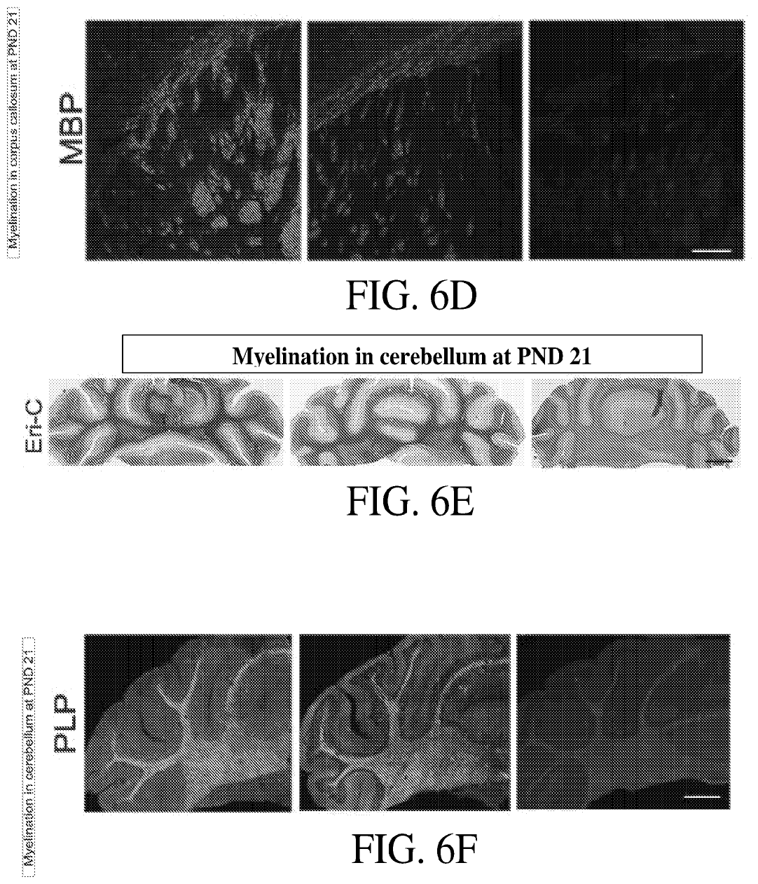

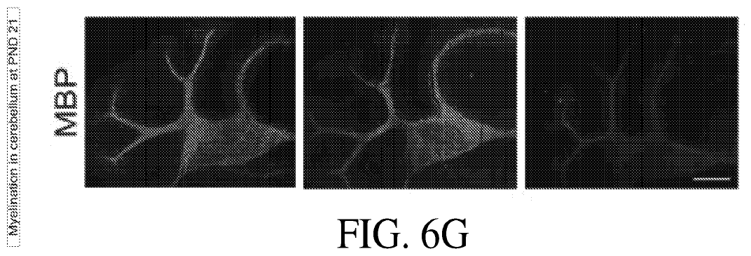

Electron microscope examination of optic nerve sections reveal evidence for unmyelinated and hypomyelinated axons in Tubb4a^{D249N/D249N} mice compared to WT control mice starting at P21 (data not shown) and worsening by the end stage time (FIGS. 5A-3C and 5H-5J) especially noticeable at higher magnification as indicated by the asterisks (FIG. 5J). In addition, vacuolated and degenerating axons (blue asterisks) engulfed by macrophages are observed in Tubb4a^{D249N/D249N} tissue at end stage (FIG. 5D). Interestingly, the g-ratio, which measures myelin thickness of axon is significantly different not only in Tubb4a^{D249N/D249N} mice (p<0.001; 0.914±0.004) but also in Tubb4a^{D249N} optic nerve (p<0.001; 0.859±0.006) compared to WT mice (FIG. 5E, 0.802±0.005). Quantification of myelin thickness measured by plotting g-ratio as a function of axon diameter (FIG. 5F) indicates that myelin sheath development is arrested in Tubb4a^{D249N} and Tubb4a^{D249N/D249N} mice. Further, compared to control mice (0.945±0.024), a significant decrease in axon caliber is noted in Tubb4a^{D249N/D249N} optic nerve tissues (FIG. 5G, p<0.05; 0.87±0.034) indicating is loss of larger axons in Tubb4a^{D249N/D249N} mice at end stage. TEM sections of spinal cord also shows a dramatic loss of myelin in Tubb4a^{D249N/D249N} mice (FIG. 4A-4F) in the ventral white matter with ongoing engulfment of axons by macrophages (FIG. 4F). However, no loss of large motor neurons was examined in the ventral horn of the spinal cord between the different groups (data not shown).

### Tubb4a^{D249N/D249N} Mice Demonstrate Neuronal Loss in Striatum and Cerebellum at End-Stage.

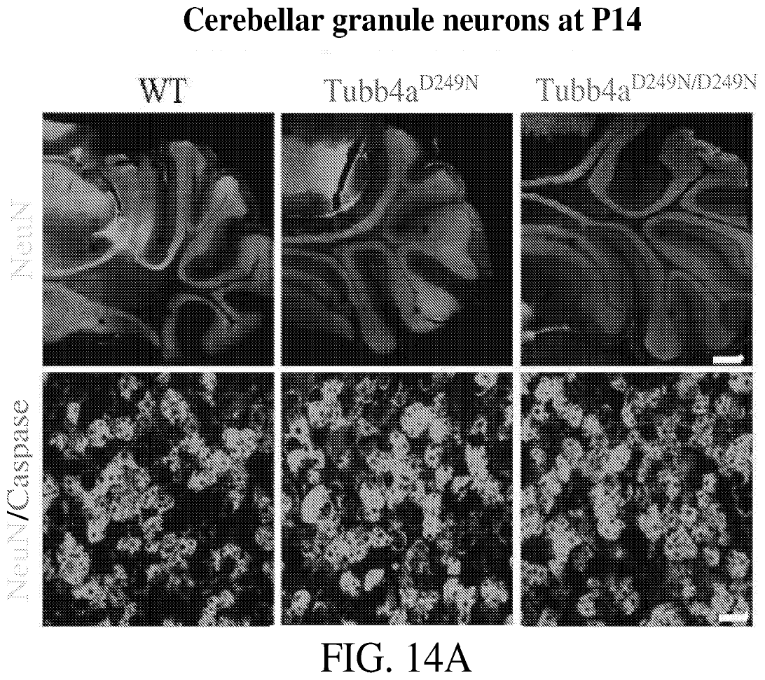

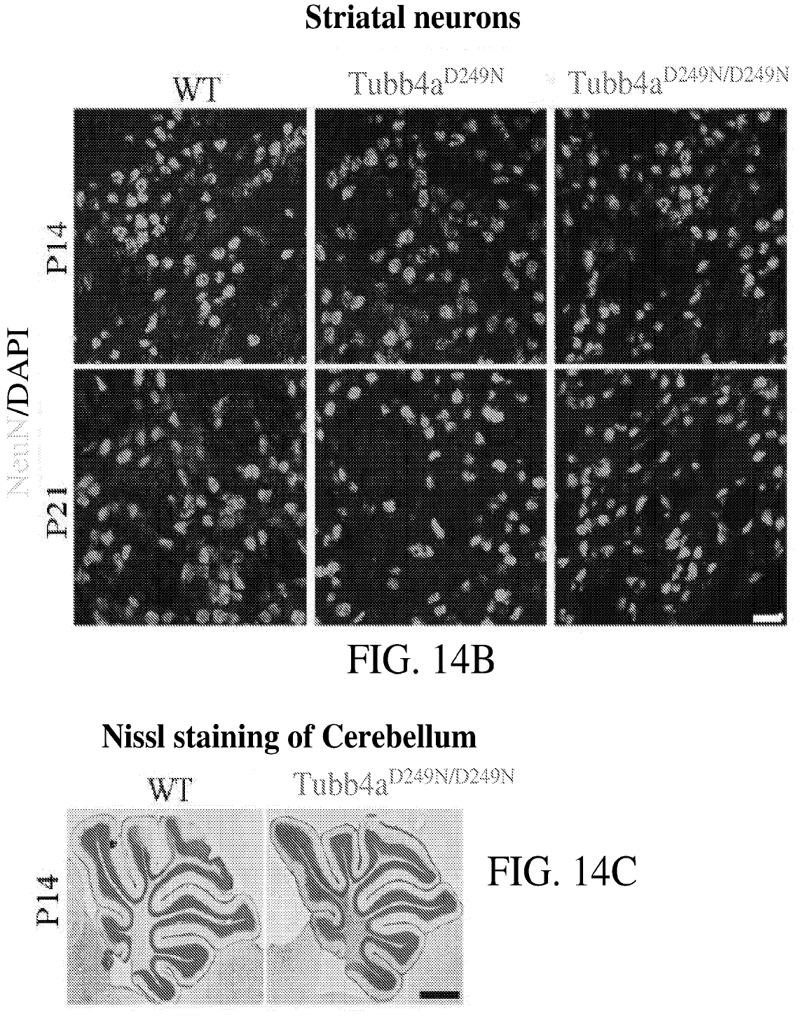

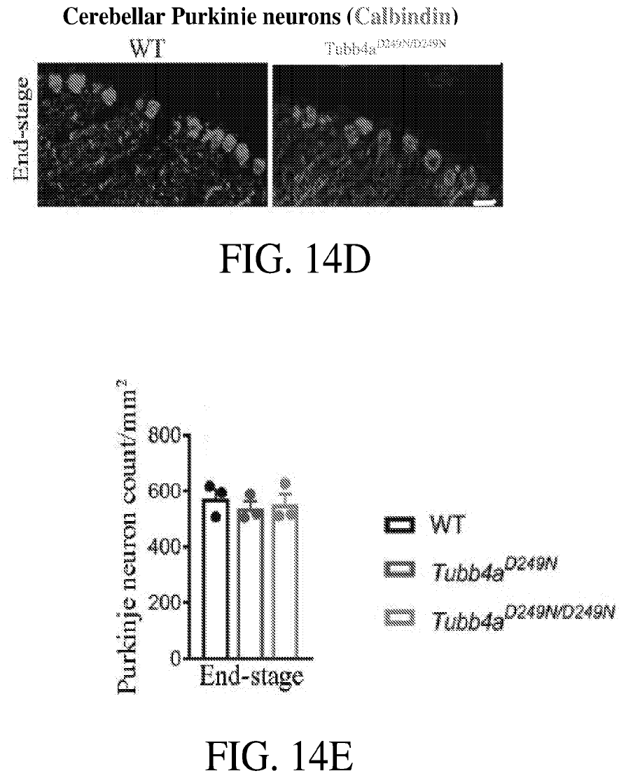

Pathologic specimens from individual affected by H-ABC demonstrate neuronal loss in basal ganglia and cerebellar granular cell layers (4, 10). Tubb4a^{D249N} and Tubb4a^{D249N/D249N} mice show signs of neuronal loss in striatum and cerebellum by immunostaining with NeuN and Nissl stain at P14 (FIG. 14A), P21 and end-stage (FIG. 9C).

At P14 and P21 time points (FIG. 14D), NeuN counts in the striatum for Tubb4a^{D249N} and Tubb4a^{D249N/D249N} are comparable to that of WT controls, however, Tubb4a^{D249N/D249N} mice demonstrate significant striatal neuronal loss (p<0.01, FIG. 9J; 183±8.44 vs 246±28.51) at end-stages. Nissl staining on the cerebellar sections of Tubb4a^{D249N/D249N} mice reveal a severe progressive loss of the granular neuron layer from P21 to end-stage and a remarkable decrease in cerebellar volume (FIG. 9C). Tubb4a^{D249N/D249N} mice display comparable granular neuronal counts at P14 compared to WT mice (FIG. 14B), however, a dramatic and progressive granular neuron loss is observed at P21 (212±6.71 vs 312±4.30) and end-stage (p<0.001, FIG. 9D-E; 57±4.7 vs 262±14.85). In addition, a significant increase in number of Caspase 3 positive cells co-localized with NeuN occurs at P21 (11±3.99 vs 0.3±0.16) and the end-stage (13.5±0.38 vs 0.75±0.38), suggesting cell apoptosis (p<0.001, FIG. 9F-G). Further, to evaluate if there is loss of other neuronal populations in the cerebellum, we evaluated Purkinje neurons by calbindin immunostaining, but Tubb4a^{D249N/D249N} and WT mice (FIGS. 14C and 14D) were similar.

Cell-Autonomous Effects of Tubb4a^{D249N} and Tubb4a^{D249N/D249N} Mutation in Oligodendrocytes

We studied the cell autonomous effect of Tubb4a mutation in OLs using in vitro cultures derived from WT control, Tubb4a^{D249N} and Tubb4a^{D249N/D249N} mice. O4+ (pre-myelinating marker) OPCs were isolated from these mice and then differentiated towards an OL fate. The cells were examined for PLP as a marker for mature OL co-localized with Olig2, a pan OL lineage marker. A significant decrease in the number of mature PLP+ OLs is examined (FIG. 11A-C) in Tubb4a^{D249N} mice (p<0.05, 72.12%±8.99) and Tubb4a^{D249N/D249N} mice (p<0.01, 55.23%±4.97) compared to mature OLs derived from WT mice (FIG. 11E). However, the total number of Olig2+ cells are similar in all the groups (FIG. 11D), resulting in a significant reduction in the proportion of mature OLs from the total cells committed to the OL lineage (PLP+/Olig2 cells, FIG. 11F) in Tubb4a^{D249N} (p<0.05, 55.34%±6.83) and Tubb4a^{D249N/D249N} mice (p<0.05, 56.04%±5.39) compared to WT mice (p<0.05, 90.5%±14.18). These results overall reflect similar changes as seen in vivo in the mouse tissues and support a cell autonomous contribution of Tubb4a^{D249N} mutation in the development of OL lineage cells.

### Cell-Autonomous Effects of Tubb4a^{D249N/D249N} Mutation in Neurons

We also examined the cell autonomous effect of Tubb4a mutation in cortical neurons using in vitro cultures derived from WT control, Tubb4a^{D249N} and Tubb4a^{D249N/D249N} mice. We analyzed the number of neurons labeled with Tuj1 and MAP2 stains at one-week post-plating for survival analysis and observed that Tubb4a^{D249N/D249N} neurons display a significant decrease in survival relative to WT neurons (FIGS. 11G, 6H, 6I, p<0.01; 69.53%±4.8). Tubb4a^{D249N} neurons do not show any difference in neuronal survival compared to WT neurons. Next, it was assessed if neuronal health and morphology is altered due to impact of the tubulin mutation on axonal outgrowth and dendritic branching. The axon length in neurons from Tubb4a^{D249N} mice is shorter compared to WT neurons (FIG. 11J, 155.8±24.42 μm versus 187.5±23.29 m) and significantly shorter in Tubb4a^{D249N/D249N} neurons than WT neurite length (p<0.05, 117.7±10.18 m). Similarly, we investigated dendritic branching in neurons (FIG. 11K) and observed the total dendritic length in Tubb4a^{D249N/D249N} neurons is significantly shorter compared to WT neurons (p<0.001, 27.43±1.53 μm versus 41.26±4.01 m) with no significant change in in Tubb4a^{D249N} neurons (31.31±3.81 m). These morphological studies indicate that H-ABC associated Tubb4a^{D249N/D249N} affects neuronal structure and formation.

Tubb4a^{D249N} and Tubb4a^{D249N/D249N} Leads to Unstable Microtubule Dynamics in Neurons

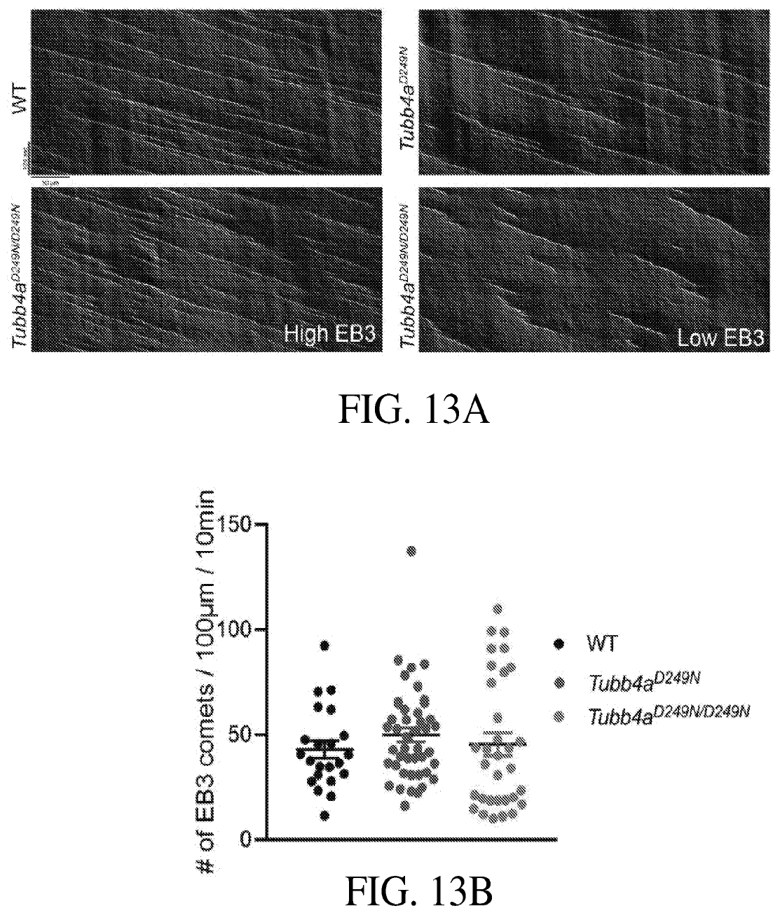

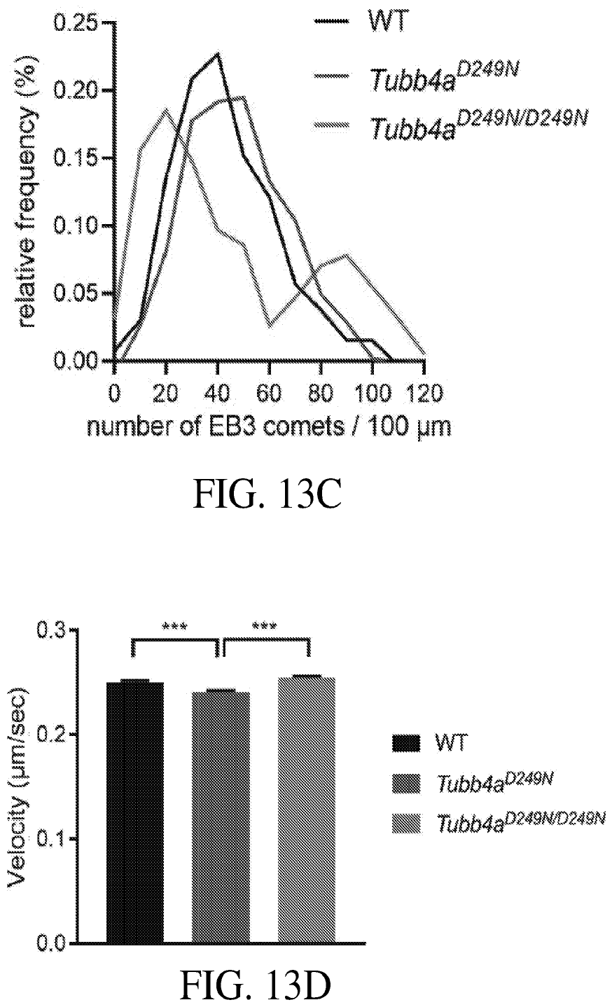

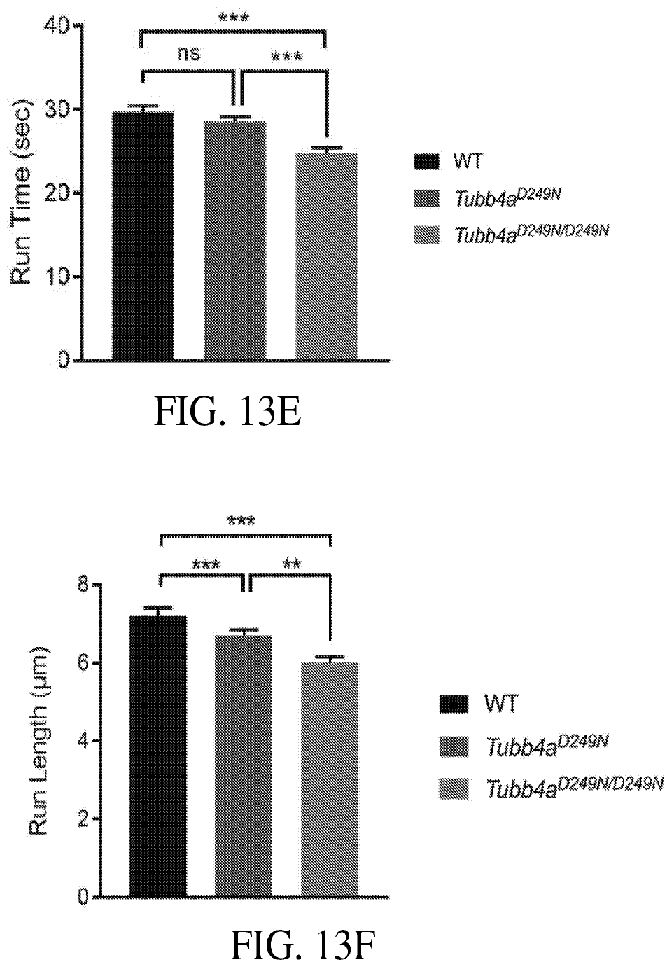

In addition to morphological studies, we conducted functional studies to asses if Tubb4a mutation affects microtubules (MT) dynamics in cortical neurons derived from WT, Tubb4a^{D249N} and Tubb4a^{D249N/D249N} mice. The neurons were transfected with the MT plus end binding protein EB3-mCherry to image the growing end of MTs at 1-week post-plating. Kymographs were generated from time lapse videos to assess EB3 comets in distal axons (FIG. 13A). While the number of EB3 comets are not significantly different in neurons from WT, Tubb4a^{D249N} and Tubb4a^{D249N/D249N} mice (FIG. 13B), interestingly the Tubb4a^{D249N/D249N} neurons display distinct populations with a high and a low expressing number of EB3 comets (FIG. 13C). The overall velocity of EB3 comets in neurons is fairly similar between the different groups (FIG. 13D; WT—0.25±0.049 μm/s, Tubb4a^{D249N}—0.241±0.055 μm/s, Tubb4a^{D249N/D249N}—0.254±0.066 μm/s) indicating similar rate of polymerization.

The average run time for these EB3 comets (FIG. 13E) in Tubb4a^{D249N/D249N} neurons is significantly shorter (p<0.001, 24.86±0.59 secs) compared to WT neurons (29.67±0.79 secs) and Tubb4a^{D249N} neurons (28.55±0.58 secs). Similarly, the average run length of EB3 comets in Tubb4a^{D249N/D249N} neurons (6.0±0.146 μm) and Tubb4aD249N neurons (6.7±0.14 μm) is significantly shorter than WT neurons (7.20±0.19 μm, p<0.001, FIG. 13F). Thus, the MT dynamics are drastically and significantly altered in Tubb4a^{D249N/D249N} neurons confirming a functional cell autonomous effect in neurons due to presence of mutation in Tubb4a gene.

### Discussion:

The mouse model described herein recapitulates the behavioral phenotypic features of H-ABC and exhibits deficits in motor function and gait. We have further validated the histologic phenotype of this model which includes the hallmark pathology of H-ABC including developmental loss of myelin, severe cerebellar atrophy and striatal neuronal loss.

Since TUBB4A mutations were associated with H1-ABC in 2013 (24), numerous other mutations in the TUBB4A gene have been identified (10, 20). The p.Asp249Asn (D249N) variant has been closely tied to the classic features of H-ABC, with a broader phenotype associated with other mutations. Unfortunately, to date, there are no therapeutic approaches available. The taeip rat model has been reported to harbor a Tubb4a mutation (homozygous p.Ala302Thr). however, the model lacks cerebellar and striatal atrophy (13, 25). In this study, we sought to fully model H-ABC by developing a Crispr-Cas9 transgenic model of the classical mutation (Tubb4a^{D249N}) Notably, this approach can be adapted to any of the nucleic acids encoding mutated Tubb4A listed in Table 1. Homozygous mice, as in the taeip rat, are necessary to show an early onset phenotype, however, heterozygous mutations as are present in humans, also show later onset disease. One potential explanation for species difference is dosage sensitivity, resulting in a dissimilar penetrance of phenotypes. This has been reported for the taiep rat model (13); in addition, there are several other reported genes such as GATA3 (26), TBX1 (27), GLI3 (28) where, heterozygous mutations are present in humans but homozygous mice have similar phenotypic expression to the human disease.

The Tubb4a^{D249N/D249N} mice show tremulous behavior starting from ˜P9 and exhibit deficits in motor-developmental skills and gait consistent with cerebellar ataxia and tremor as seen in H-ABC affected individuals. Over time, there is a severe decline in their motor function consistent with the onset of dystonia. Tubb4a^{D249N/D249N} mice display significant decrease in weight and survival at ˜P37 presumably as they are not able to feed themselves due to severe dystonia and spastic ataxia. Heterozygous Tubb4a^{D249N} mice show no early evidence of the severe behavioral and neuropathological phenotype seen in their homozygous counterparts, but have myelin loss at one year with no discernible behavioral phenotype.

In neuropathology, Tubb4a^{D249N/D249N} mice demonstrate diminished and progressive developmental loss of myelin, consistent with a mixed hypomyelination and dysmyelination over time, which may contribute to the early onset tremor as seen in Shiverer (29), Shimild (30) and Jimpy mice (31). Tubb4a^{D249N/D249N} mice show severe oligodendrocyte (OL) loss at P14. Myelin loss is likely attributable to OLs death as evidenced by decreased numbers of OLs based on histology. Additionally, based on the high expression of Tubb4a in OLs (11) and caspase staining, we propose that mutation in Tubb4a contributes to OL death. OPC counts appear to be preserved in Tubb4a^{D249N/D249N} mice suggesting that the mutation in Tubb4a may also affect differentiation of OLs from OPC's. Together, both of these mechanisms contribute to the combined hypomyelination and dysmyelination seen in this model. Tubb4a^{D249N/D249N} mice also show evidence of severe loss of cerebellar granular neurons at P21 and significant striatal neuron degeneration after ˜P37. This is consistent with reported pathology in individuals affected by H-ABC (9, 32). However, Purkinje neurons appear grossly intact. Drastic cerebellar changes overtime may underlie the progressive gait abnormalities, ataxia and motor dysfunction seen in both this mouse model and affected individuals. The somewhat milder defects in striatal neurons may be related to the variable expression of Tubb4a, where Tubb4a expression is relatively higher in cerebellum than striatum (2). As increased numbers of TUBB4A mutations have been reported (9, 33, 34), it is becoming recognized that mutation specific cellular effects, with independent involvement of the striatum, myelinating cells and cerebellum may be responsible for the wide phenotypic variability seen in this condition (4, 9).

Tubb4a^{D249N/D249N} mice provide the first model to fully enable molecular dissection of the relevant cellular subtypes affected in H-ABC. The cellular effects observed in neurons and OLs here could be occurring as independent contributions or these could be an additive non-cell autonomous effect. Examining the vulnerabilities of each cell population will be important in developing effective treatment options for individuals with H-ABC.

To dissect this, we studied the cell autonomous effects of neurons and OLs using a reductionist cell culture model. OPCs isolated from Tubb4a^{D249N} and Tubb4a^{D249N/D249N} mice differentiated less efficiently into OLs as opposed to WT control mice, confirming a cell autonomous effect of Tubb4a^{D249N} in OLs, also reflected in the hypomyelination and dysmyelination observed in vivo. As the total number of Olig2 labeled cells are similar but the number of mature OLs are decreased in Tubb4a^{D249N} and Tubb4a^{D249N/D249N} mice, this indicates deficits in differentiation of OPCs towards a mature OL fate due to Tubb4a mutations. Further delineation of the non-cell autonomous effects of Tubb4a mutations will require full genetic exploration using conditional transgenic mouse models.

Some evidence for microtubule (MT) dysfunction and associated OL maturation due to TUBB4A mutation comes from studies conducted in the taeip rat model. The taeip rat display an accumulation of microtubules in OLs along with perinuclear localization of RNA for PLP, MAG and MBP myelin genes and which was further attributed to increased activity of the motor protein dynein in OLs for MBP trafficking (35). These key myelin proteins need to be transported along MT from the OL cell body to the periphery for myelin synthesis. Given the complexity of OL processes and myelin sheath development, it can be foreseen that inefficient delivery of cargo along MTs contributes to the decreased maturation and complexity of Tubb4a^{D249N/D249N} OLs.

To dissect the cell autonomous effects of neurons, we similarly studied cortical neurons in culture from Tubb4a^{D249N} and Tubb4a^{D249N/D249N} mice, demonstrating decreased survival and stunted axonal and dendritic branching. Functional studies reveal shorter distance and time of polymerization in MT plus end, indicative of unstable MT dynamics in neurons harboring Tubb4a^{D249N} and Tubb4a^{D249N/D249N} mutations. MTs are integral to neuronal development and function contributing to neuronal structure, polarity, growth cone dynamics, intracellular transport (36) and mutant Tubb4a protein potentially affects these key functions. A number of mutations in α-tubulin and β-tubulin are attributed to a spectrum of neurological disorders characterized by deficits in neuronal migration, differentiation and axon guidance (37-39). Similar to our studies, Tubb3 mutations in mouse cortical neurons and yeast displayed altered MT dynamics and disrupted interaction of MT with kinesin motors (40). Studies in Parkinson's disease (41) and ALS (42) indicated that altered MT dynamics impair axonal transport crucial for active transport and it is feasible that Tubb4a^{D249N/D249N} results in inefficient MT dynamics hampering the transport of cargo required for axon elongation and dendritic branching. While these studies are conducted in cortical neurons from Tubb4a^{D249N/D249N} mice, similar or more dramatic effect is expected in striatal or cerebellar granular neurons.

Specific residues on the MT surface regulate a number of protein interactions and changes in these residues affect their function leading to a range of neurological disorders (39). MT dynamics can be altered due to post-translational modifications (PTM) such as tyrosination, acetylation, polyamination, glutamylation, glycylation and glutathyonilation (43) that confer stability to MTs. Since the location of p.Asp249Asn mutation in the functional domain of TUBB4A is thought to affect the stability of assembled microtubules (4), it is feasible that changes in PTM could be a contributing factor to Tubb4a^{D249N} mediated MT abnormalities. PTM can further alter the binding of motor proteins such as kinesin, dynein and MT-associated proteins (MAPs) such as Tau and doublecortin crucial for organelle and molecular transport of cargo (36). However, the exact mechanism through which Tubb4a mutation impacts microtubules function remains unknown and needs to be explored.

TUBB4A heterozygous mutations cause a spectrum of brain malformations, suggesting that Tubb4a might have a critical role in neuronal and glial function. However, the Tubb4a knock out (KO) mouse model suggests that Tubb4a might be redundant for the brain function. Tubb4a KO mice with LacZ expression (44) are available at the KOMP repository with partial phenotypic data on the world wide web at .mousephenotype.org/data/genes/MGI:10784#section-associations. Homozygous Tubb4a KO mice show normal embryonic development and grow fine with the normal weights as relative to WTs. The phenotypic lac Z expression data shows that the nervous system appears normal with no discernible cerebellar neuronal loss. The present data suggest that TUBB4A might not be essential for brain development and function, implicating a toxic gain of function effect of TUBB4A mutations in neurons and oligodendrocytes. Further, our previous in vitro cellular work (45) and a recent study in induced pluripotent stem cell-derived neurons (46) reported that the mutation in D249N causes alteration in polymerization rate of tubulins, thus, supporting dominant toxic gain of function effects in the H-ABC disease pathogenesis.

H-ABC is a devastating, progressive and disabling pediatric disorder for which there are no therapeutic strategies available. Tubb4a^{D249/D249NN} mice are the first to develop the molecular, behavioral and neurodegenerative features of classical H-ABC disease. Mice show both neuronal and oligodendroglial defects. Taken together, these data support a model in which altered microtubules are critical drivers of disease pathogenesis. These mice provide a key tool that can be used to tease out the molecular mechanisms of this complex disease involving neurons and glia and test the efficacy of therapeutic strategies.

### REFERENCES

1. Simons C, Wolf N I, McNeil N, Caldovic L, Devaney J M, Takanohashi A, Crawford J, Ru K, Grimmond S M, Miller D, et al. A de novo mutation in the beta-tubulin gene TUBB4A results in the leukoencephalopathy hypomyelination with atrophy of the basal ganglia and cerebellum. Am J Hum Genet. 2013; 92(5):767-73.
2. Hersheson J, Mencacci N E, Davis M, MacDonald N, Trabzuni D, Ryten M, Pittman A, Paudel R, Kara E, Fawcett K, et al. Mutations in the autoregulatory domain of beta-tubulin 4a cause hereditary dystonia. Ann Neurol. 2013; 73(4):546-53.
3. van der Knaap M S, Linnankivi T, Paetau A, Feigenbaum A, Wakusawa K, Haginoya K, Kohler W, Henneke M, Dinopoulos A, Grattan-Smith P, et al. Hypomyelination with atrophy of the basal ganglia and cerebellum: follow-up and pathology. Neurology. 2007; 69(2):166-71.
4. Curiel J, Rodriguez Bey G, Takanohashi A, Bugiani M, Fu X, Wolf N I, Nmezi B, Schiffmann R, Bugaighis M, Pierson T, et al. TUBB4A mutations result in specific neuronal and oligodendrocytic defects that closely match clinically distinct phenotypes. Hum Mol Genet. 2017; 26(22):4506-18.
5. Blumkin L, Halevy A, Ben-Ami-Raichman D, Dahari D, Haviv A, Sarit C, Lev D, van der Knaap M S, Lerman-Sagie T, and Leshinsky-Silver E. Expansion of the spectrum of TUBB4A-related disorders: a new phenotype associated with a novel mutation in the TUBB4A gene. Neurogenetics. 2014; 15(2):107-13.
6. Ferreira C, Poretti A, Cohen J, Hamosh A, and Naidu S. Novel TUBB4A mutations and expansion of the neuroimaging phenotype of hypomyelination with atrophy of the basal ganglia and cerebellum (H-ABC). Am J Med Genet A. 2014; 164A(7):1802-7.
7. Purnell S M, Bleyl S B, and Bonkowsky J L. Clinical exome sequencing identifies a novel TUBB4A mutation in a child with static hypomyelinating leukodystrophy. Pediatr Neurol. 2014; 50(6):608-11.
8. Miyatake S, Osaka H, Shiina M, Sasaki M, Takanashi J, Haginoya K, Wada T, Morimoto M, Ando N, Ikuta Y, et al. Expanding the phenotypic spectrum of TUBB4A-associated hypomyelinating leukoencephalopathies. Neurology. 2014; 82(24):2230-7.
9. Pizzino A, Pierson T M, Guo Y, Helman G, Fortini S, Guerrero K, Saitta S, Murphy J L, Padiath Q, Xie Y, et al. TUBB4A de novo mutations cause isolated hypomyelination. Neurology. 2014; 83(10):898-902.
10. Nahhas N C A, Hamilton A, Curiel J, Simons C, van der Knaap M S and Vanderver A. In: Adam M P A H, Pagon R A ed. GeneReviews® [Internet]. Seattle (Wash.): University of Washington, Seattle; 2016.
11. Zhang Y, Chen K, Sloan S A, Bennett M L, Scholze A R, O'Keeffe S, Phatnani H P, Guarnieri P, Caneda C, Ruderisch N, et al. An RNA-sequencing transcriptome and splicing database of glia, neurons, and vascular cells of the cerebral cortex. J Neurosci. 2014; 34(36):11929-47.
12. Duncan I D, Bugiani M, Radcliff A B, Moran J J, Lopez-Anido C, Duong P, August B K, Wolf N I, van der Knaap M S, and Svaren J. A mutation in the Tubb4a gene leads to microtubule accumulation with hypomyelination and demyelination. Ann Neurol. 2017; 81(5):690-702.
13. Li F Y, Song J, and Duncan I D. Mapping of taiep rat phenotype to rat Chromosome 9. Mamm Genome. 2003; 14(10):703-5.
14. Feather-Schussler D N, and Ferguson T S. A Battery of Motor Tests in a Neonatal Mouse Model of Cerebral Palsy. J Vis Exp. 2016117).
15. Shiotsuki H, Yoshimi K, Shimo Y, Funayama M, Takamatsu Y, Ikeda K, Takahashi R, Kitazawa S, and Hattori N. A rotarod test for evaluation of motor skill learning. J Neurosci Methods. 2010; 189(2):180-5.
16. Sahinkaya F R, Milich L M, and McTigue D M. Changes in NG2 cells and oligodendrocytes in a new model of intraspinal hemorrhage. Exp Neurol. 2014; 255(113-26.
17. Lancaster E, Li J, Hanania T, Liem R, Scheideler M A, and Scherer S S. Myelinated axons fail to develop properly in a genetically authentic mouse model of Charcot-Marie-Tooth disease type 2E. Exp Neurol. 2018; 308(13-25.
18. Guedes-Dias P, Nirschl J J, Abreu N, Tokito M K, Janke C, Magiera M M, and Holzbaur E L F. Kinesin-3 Responds to Local Microtubule Dynamics to Target Synaptic Cargo Delivery to the Presynapse. Curr Biol. 2019; 29(2):268-82 e8.
19. Mangeol P, Prevo B, and Peterman E J. KymographClear and KymographDirect: two tools for the automated quantitative analysis of molecular and cellular dynamics using kymographs. Mol Biol Cell. 2016; 27(12):1948-57.
20. Hamilton E M, Polder E, Vanderver A, Naidu S, Schiffmann R, Fisher K, Raguz A B, Blumkin L, Group H A R, van Berkel C G, et al. Hypomyelination with atrophy of the basal ganglia and cerebellum: further delineation of the phenotype and genotype-phenotype correlation. Brain. 2014; 137(Pt 7):1921-30.
21. Wenzel D G, and Lal H. The relative reliability of the escape reaction and righting-reflex sleeping times in the mouse. J Am Pharm Assoc Am Pharm Assoc. 1959; 48(2):90-1.
22. Rahn E J, Iannitti T, Donahue R R, and Taylor B K. Sex differences in a mouse model of multiple sclerosis: neuropathic pain behavior in females but not males and protection from neurological deficits during proestrus. Biol Sex Differ. 2014; 5(1):4.
23. Baumann N, and Pham-Dinh D. Biology of oligodendrocyte and myelin in the mammalian central nervous system. Physiol Rev. 2001; 81(2):871-927.
24. Simons C, Wolf N I, McNeil N, Caldovic L, Devaney J M, Takanohashi A, Crawford J, Ru K, Grimmond S M, Miller D, et al. A de novo mutation in the beta-tubulin gene TUBB4A results in the leukoencephalopathy hypomyelination with atrophy of the basal ganglia and cerebellum. American journal of human genetics. 2013; 92(5):767-73.
25. Duncan I D, Bugiani M, Radcliff A B, Moran J J, Lopez-Anido C, Duong P, August B K, Wolf N I, van der Knaap M S, and Svaren J. A mutation in the Tubb4a gene leads to microtubule accumulation with hypomyelination and demyelination. Annals of neurology. 2017; 81(5):690-702.
26. Lim K C, Lakshmanan G, Crawford S E, Gu Y, Grosveld F, and Engel J D. Gata3 loss leads to embryonic lethality due to noradrenaline deficiency of the sympathetic nervous system. Nat Genet. 2000; 25(2):209-12.
27. Jerome L A, and Papaioannou V E. DiGeorge syndrome phenotype in mice mutant for the T-box gene, Tbx1. Nat Genet. 2001; 27(3):286-91.
28. Bose J, Grotewold L, and Ruther U. Pallister-Hall syndrome phenotype in mice mutant for Gli3. Hum Mol Genet. 2002; 11(9):1129-35.
29. Biddle F, March E, and Miller J. Research news. Mouse News Lett. 1973; 48(24).
30. Readhead C, and Hood L. The dysmyelinating mouse mutations shiverer (shi) and myelin deficient (shimld). Behav Genet. 1990; 20(2):213-34.
31. Nave K A, Lai C, Bloom F E, and Milner R J. Jimpy mutant mouse: a 74-base deletion in the mRNA for myelin proteolipid protein and evidence for a primary defect in RNA splicing. Proc Natl Acad Sci USA. 1986; 83(23):9264-8.
32. van der Knaap M S, Naidu S, Pouwels P J, Bonavita S, van Coster R, Lagae L, Sperner J, Surtees R, Schiffmann R, and Valk J. New syndrome characterized by hypomyelination with atrophy of the basal ganglia and cerebellum. AJNR Am J Neuroradiol. 2002; 23(9):1466-74.
33. Lu Y, Ondo Y, Shimojima K, Osaka H, and Yamamoto T. A novel TUBB4A mutation G96R identified in a patient with hypomyelinating leukodystrophy onset beyond adolescence. Human Genome Variation. 2017; 4(17035.
34. Tonduti D, Aiello C, Renaldo F, Dorboz I, Saaman S, Rodriguez D, Fettah H, Elmaleh M, Biancheri R, Barresi S, et al. TUBB4A-related hypomyelinating leukodystrophy: New insights from a series of 12 patients. European journal of paediatric neurology: EJPN: official journal of the European Paediatric Neurology Society. 2016; 20(2):323-30.
35. Song J, Carson J H, Barbarese E, Li F Y, and Duncan I D. RNA transport in oligodendrocytes from the taiep mutant rat. Mol Cell Neurosci. 2003; 24(4):926-38.
36. Clark J A, Yeaman E J, Blizzard C A, Chuckowree J A, and Dickson T C. A Case for Microtubule Vulnerability in Amyotrophic Lateral Sclerosis: Altered Dynamics During Disease. Front Cell Neurosci. 2016; 10(204.
37. Keays D A, Tian G, Poirier K, Huang G J, Siebold C, Cleak J, Oliver P L, Fray M, Harvey R J, Molnar Z, et al. Mutations in alpha-tubulin cause abnormal neuronal migration in mice and lissencephaly in humans. Cell. 2007; 128(1):45-57.
38. Tischfield M A, Cederquist G Y, Gupta M L, Jr., and Engle E C. Phenotypic spectrum of the tubulin-related disorders and functional implications of disease-causing mutations. Curr Opin Genet Dev. 2011; 21(3):286-94.
39. Chakraborti S, Natarajan K, Curiel J, Janke C, and Liu J. The emerging role of the tubulin code: From the tubulin molecule to neuronal function and disease. Cytoskeleton (Hoboken). 2016; 73(10):521-50.
40. Tischfield M A, Baris H N, Wu C, Rudolph G, Van Maldergem L, He W, Chan W M, Andrews C, Demer J L, Robertson R L, et al. Human TUBB3 mutations perturb microtubule dynamics, kinesin interactions, and axon guidance. Cell. 2010; 140(1):74-87.
41. Cartelli D, Ronchi C, Maggioni M G, Rodighiero S, Giavini E, and Cappelletti G.

Microtubule dysfunction precedes transport impairment and mitochondria damage in MPP+-induced neurodegeneration. J Neurochem. 2010; 115(1):247-58.
42. Fanara P, Banerjee J, Hueck R V, Harper M R, Awada M, Turner H, Husted K H, Brandt R, and Hellerstein M K. Stabilization of hyperdynamic microtubules is neuroprotective in amyotrophic lateral sclerosis. J Biol Chem. 2007; 282(32):23465-72.
43. Janke C. The tubulin code: molecular components, readout mechanisms, and functions. J Cell Biol. 2014; 206(4):461-72.
44. Skarnes W C, Rosen B, West A P, Koutsourakis M, Bushell W, Iyer V, Mujica A O, Thomas M, Harrow J, Cox T, et al. A conditional knockout resource for the genome-wide study of mouse gene function. Nature. 2011; 474(7351):337-42.
45. Curiel J, Rodriguez Bey G, Takanohashi A, Bugiani M, Fu X, Wolf N I, Nmezi B, Schiffmann R, Bugaighis M, Pierson T, et al. TUBB4A mutations result in specific neuronal and oligodendrocytic defects that closely match clinically distinct phenotypes. Human molecular genetics. 2017; 26(22):4506-18.
46. Vulinovic F, Krajka V, Hausrat T J, Seibler P, Alvarez-Fischer D, Madoev H, Park J S, Kumar K R, Sue C M, Lohmann K, et al. Motor protein binding and mitochondrial transport are altered by pathogenic TUBB4A variants. Human mutation. 2018; 39(12):1901-15.
47. Truett G E, Heeger P, Mynatt R L, Truett A A, Walker J A, and Warman M L. Preparation of PCR-quality mouse genomic DNA with hot sodium hydroxide and tris (HotSHOT). Biotechniques. 2000; 29(1):52, 4.
48. Lee Y W, Mirocha C J, Shroeder D J, and Walser M M. TDP-1, a toxic component causing tibial dyschondroplasia in broiler chickens, and trichothecenes from Fusarium roseum ‘Graminearum’. Appl Environ Microbiol. 1985; 50(1):102-7.

The following materials and methods are provided to facilitate the practice of Example II.

### Methods for iPS Culture:

Peripheral blood monocytes (PBMCs) isolated from individuals with TUBB4A mutations and unaffected individuals (controls) were reprogrammed into induced pluripotent stem cells (iPSCs). All the iPSC lines were confirmed for markers of pluripotency using flowcytometry and DNA finger printing to confirm genetic integrity of iPSC lines (Maguire et. al 2019). The iPSC clones exhibited normal karyotype throughout serial passaging and mycoplasm testing conducted was negative for all the lines.

Neuronal Differentiation of iPSCs:

Neural inductions towards a striatal medium spiny neuron (MSN) fate was conducted using a dual SMAD inhibition protocol that is previously published (Telezhkin 2016). Briefly, the iPSCs were grown in Essential 8 media with TGF-β and bFGF until they reached 70% confluency. The cells were washed with PBS and the neural induction SLI medium containing 10 μM SB431542, 1.5 μM LDN 193189, 1.5 μM IWR1 in Neurobasal medium without retinoic acid (RA). On day 4, the confluent cultures were passaged onto fresh Matrigel coated plates with a split ratio of 1:2. On day 8, cultures were passaged 1:2 and cultured in L1 medium containing 200 nM LDN193189, 1.5 μM IWR1 without RA in Neurobasal medium and the medium was changed daily. D16 iPSC derived neural progenitor cells (NPCS) were either used directly for neuronal differentiation or frozen for later differentiations. Flow cytometry was conducted on NPCs to look for early markers of differentiation such as SOX2, Pax6, FOXG1 and Nestin.

For neuronal differentiation, NPCs were dissociated using accutase and plated at 100,000 cells per well in a 24 well plate coated with Matrigel and 100 μg/ml poly-L-lysine (PLL). The d16 NPCs were plated in SCM1 medium for the first 7 days containing Advanced DMEMF12, 2 μM PD0332991, 10 μM DAPT, 0.6 mM CaCl₂), 200 μM ascorbic acid, 10 μM forskolin, 3 μM CHIR99021 and 300 μM GABA.

After day 8 plating of NPCs (or day 23 total), NPCs were cultured in SCM2 medium containing 1:1 Advanced DMEM/F-12: Neurobasal A, with RA, 2 μM PD0332991, 3 μM CHIR99021, 0.3 mM CaCl₂), 200 μM ascorbic acid, 10 ng/ml BDNF. The medium was changed every third day until they reached day 38, after which the cells were ready for experiment.

### Neuronal Cell Survival and Analysis:

Neurons grown on coverslips were fixed with 4% PFA for 20 mins and stained for Tuj1 (neuronal marker), MAP2 (dendritic marker) and specific markers for MSNs such as DARPP32, CTIP2, GABA and FOXP1. The cells were fixed at different time points post maturation (day 38, day 45, day 52) to conduct survival analysis and neuropathology by staining and imaging of coverslips with the Leica microscope. The data was analyzed in a blinded fashion and graphpad prism was used to conduct the statistical analysis. All the analysis was done using either one way or two-way ANOVA followed by Tukey post-hoc test. *p<0.05, **p<0.01, ***p<0.01.

### Example II

### TUBB4A Human iPS Cells

As discussed in the previous example, hypomyelinating atrophy of basal ganglia and cerebellum (H-ABC) is a rare leukodystrophy which our group has identified to be caused by sporadic de novo heterozygous mutations in the TUBB4A gene (Simons et al. 2013). Monoallelic mutations in TUBB4A may result in a spectrum of neurologic disorders ranging from an early onset encephalopathy to an adult-onset Dystonia type 4 (whispering dysphonia). H-ABC affected individuals are within this spectrum, presenting in the toddler years, typically with dystonia (Hersheson et al. 2013), progressive gait impairment, speech and cognitive deficits. They are further delineated from other individuals with mutations in TUBB4A by characteristic neuroimaging features: hypomyelination and atrophy of the caudate and putamen along with cerebellar atrophy (van der Knaap et al. 2007). On pathologic specimens, dorsal striatal areas and the granular layer of the cerebellum exhibit neuronal loss with axonal swelling and diffuse paucity of myelin (Curiel et al. 2017; Simons et al. 2013). Individuals with H-ABC represent about 65% of published TUBB4A mutations and are disproportionately likely to be affected by a single common mutation, p. Asp249Asn (referred to hereafter as D249N).

In the present example, we describe reprogrammed induced pluripotent stem cell (iPSC) lines from peripheral blood monocytes (PBMCs) isolated from individuals with H-ABC and other TUBB4A mutations at the CHOP stem cell core (See Table 1). In context of H-ABC, we have specifically reprogramed 3 lines of TUBB4A^{D249N} lines to differentiate them towards a striatal medium spiny neuron fate and examine their pathology. Data obtained to date indicates decreased survival (FIGS. 16A-D, **p<0.01, *** p<0.001) and clear neuropathology (FIG. 16E, *p<0.05) in the medium spiny neurons differentiated from TUBB4A^{D249N} iPSCs (red bar) compared to neurons derived from control patients (black bar). To test if TUBB4A associated pathology is through loss or gain of function, we deleted TUBB4A using CRISPR in a control patient iPSC line to test if the knock out (TUBB4A KO) lines are developmentally normal and differentiate efficiently into striatal neurons. In fact, we observed the differentiation of control versus control TUBB4A KO iPSCs are comparable (FIGS. 16F, G) indicating TUBB4A does not have a developmental role in generation of striatal neurons.

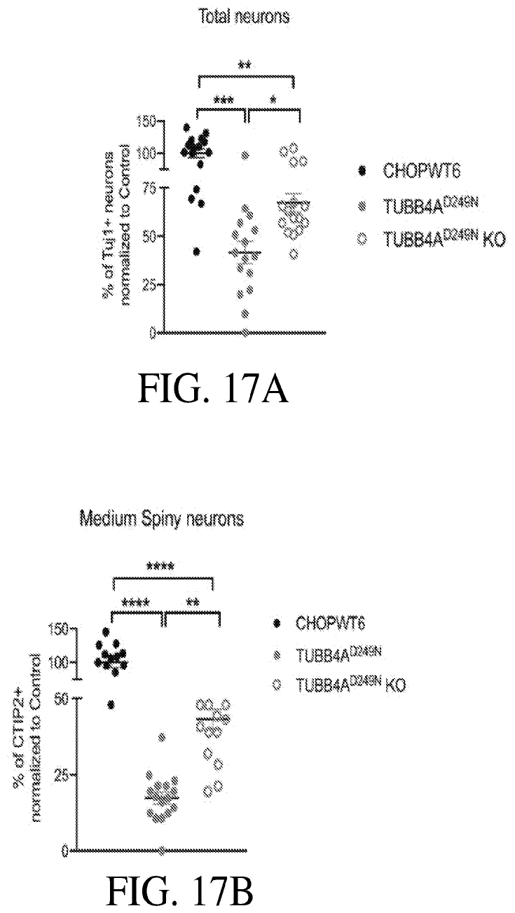

Since TUBB4A deletion did not result in loss of function, we next tested if deletion of TUBB4A in iPSCs from TUBB4A^{D249N} patients could lead to rescue of pathology and neuronal death (as seen in FIG. 16) by generating TUBB4AKO in the patient iPSCs (TUBB4A^{D249N}KG). We further differentiated the TUBB4A^{D249N} KO iPSCs towards a neuronal fate and observed there was a significant rescue in the total number of neurons (FIG. 17; *p<0.05, 67.10±4.76 versus 41.55±5.82) and also CTIP2+ MSNs (FIG. 16B;**p<0.01, 43.14±3.38 versus 17.38±2.01) in the TUBB4A^{D249N} KO compared to TUBB4A^{D249N} neurons.

These findings provide proof-of-principal for the treatment of H-ABC and the related TUBB4A-associated leukodystrophy with measures to down-regulate expression of mutant or increase wild-type TUBB4A. Therapeutic approaches employing this approach may include anti-sense oligonucleotides, RNA silencing approaches or competition against mutant TUBB4A by overexpression of wild type TUBB4A.

### Example III

### Antisense Molecules for Down Modulation of Target TUBB4A Genes

The present inventors have developed a series of antisense oligonucleotides which are effective to down modulate overall levels of TUBB4A gene expression. The approach described below enables down modulation of wild type and mutant TUBB4-A expression in a target cell of interest.

Eleven ASOs were synthesized by Integrated DNA Technologies. We performed in vitro screening of these ASOs to identify the best ASO design. Mouse Oli-neu cells were electroporated with 1 μM, 5 μM and 10 μM of ASO concentrations at 150 V in 100 μL media with 100,000 cells/well on the NEPA21 electroporation system (NEPA GENE, USA). Following electroporation, cells were transferred to a poly-L-ornithine coated plate and placed in an incubator. Forty-eight hours post-treatment, cells were washed with PBS and then RNA extraction was performed using PureLink™ RNA Mini Kit (ThermoFisher Scientific, Cat: 12183018A) according to manufacturer's instructions. After treatment with DNAase (Invitrogen), 200 ng of RNA was used for cDNA with SuperScript™ IV First-Strand Synthesis System (ThermoFisher Scientific, Cat: 18091200). The mRNA expression levels of Tubb4a, and an endogenous housekeeping gene encoding Splicing factor, arginine/serine-rich 9 (sfrs9) as a reference, were quantified using real-time PCR analysis (Tagman chemistry) on an Applied Biosystems Quanta Flex 7 (ThermoFisher Scientific, USA). The results were analyzed using the ΔΔCT method.

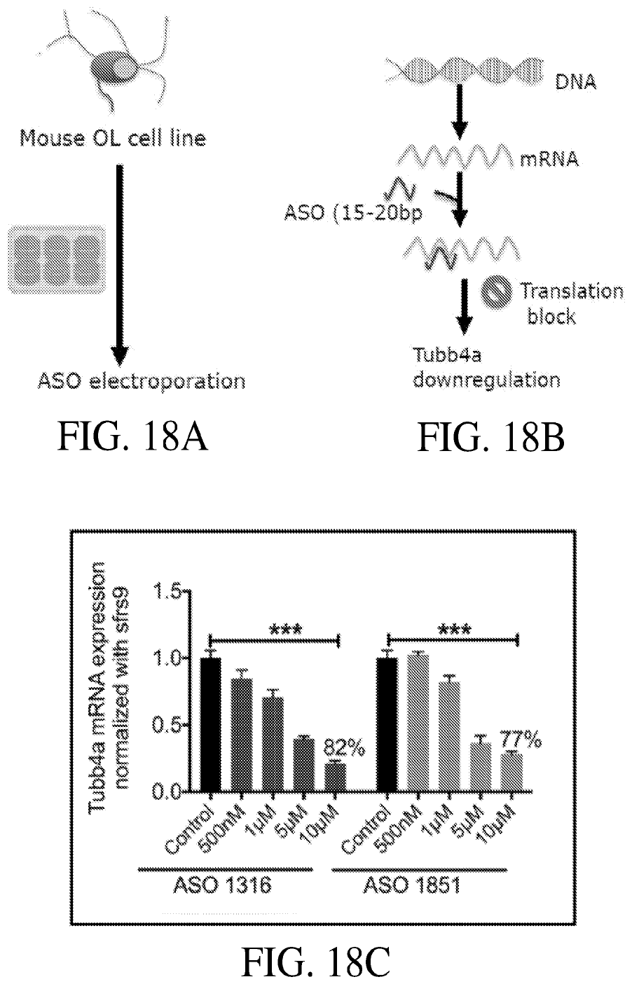

Anti-sense oligonucleotides (ASO) sequences: After electroporation, following ASO sequences showed maximal downregulation of Tubb4a at 10 μM. See FIGS. 18 and. 21A.

(SEQ ID NO: 1)
ASO 1316: Sequence: 5′+A*+C*+A*T*A*C*G*G*C*T*G*
T*C*+T*+T*+G 3′
(SEQ ID NO: 2)
ASO 1851: Sequence: 5′+G*+A*+T*C*T*A*A*G*A*A*G*
G*T*+G*+G*+A 3′
*Melting Tm is not assessed using Mg2+ or dNTP
concentration
+LNA modification

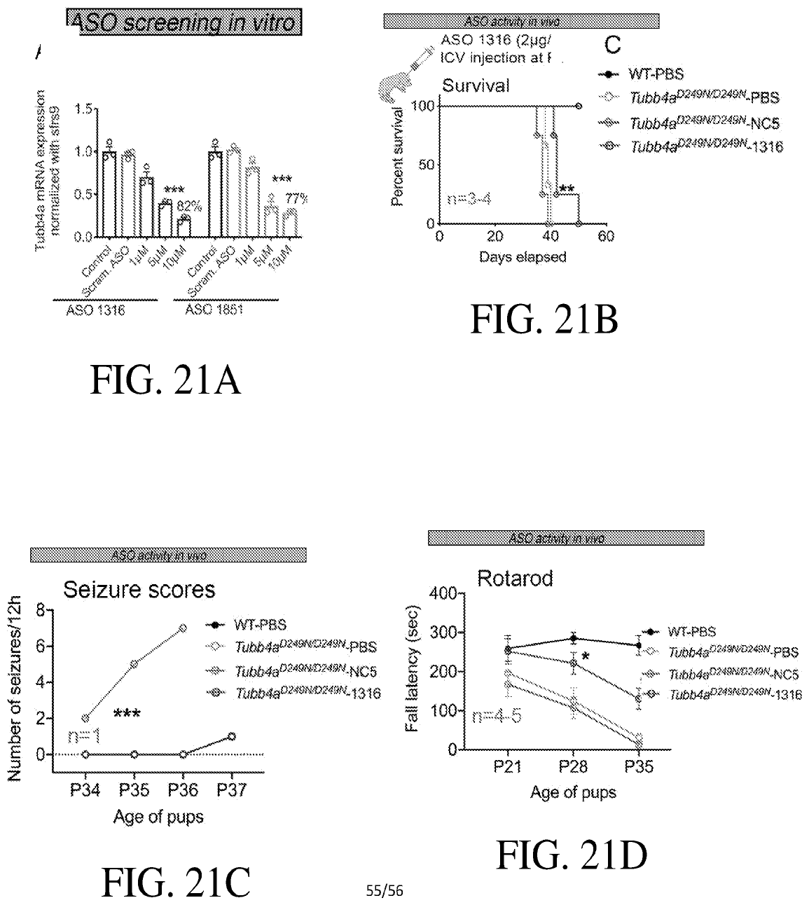

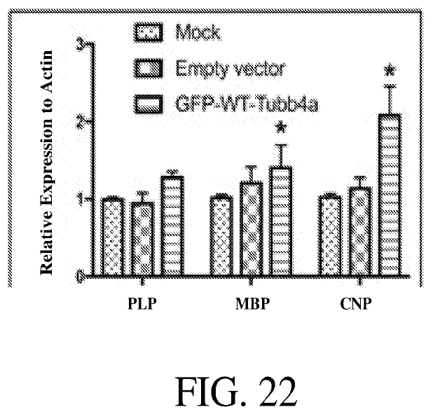

Each of the sequences listed above may optionally include one or more modified backbone linkages and/or a modified sugar. These ASOs are viable therapeutic targets for downregulation of Tubb4A. (FIG. 21) These ASO are shown to effectively downregulate Tubb4a at 0.5 μM, 1 μM, 2 μM, 5 μM, 10 μM, and 25 μM with minimal toxicity. (FIG. 21A) In fact, when treated with an ASO subjects are shown to have increased survival (FIG. 21B), reduced seizures, (FIG. 21C) and significantly improved motor function (FIG. 21D).

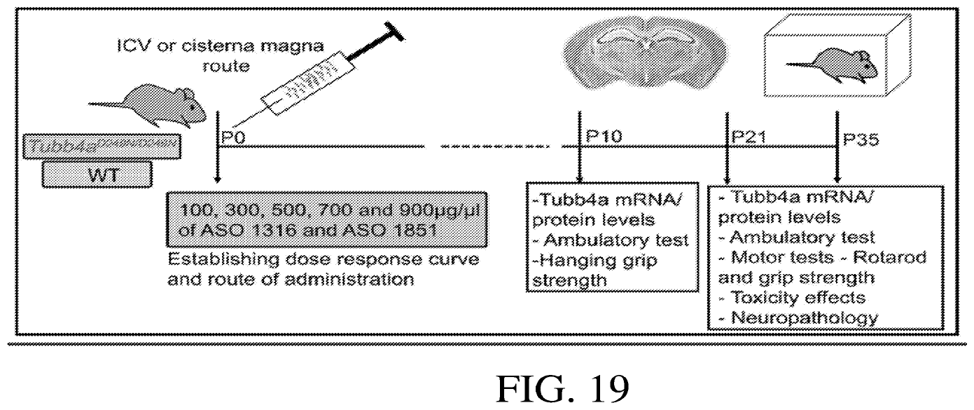

FIG. 19 shows a schematic of in vivo whole animal treatment using the antisense oligonucleotides of the invention.

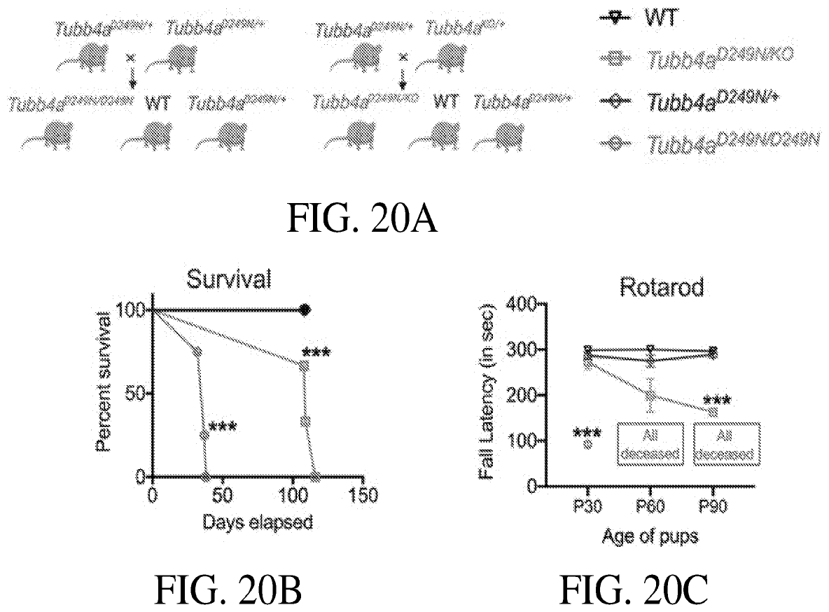

FIG. 20 shows the therapeutic efficacy of downregulating TUBB4A in an established Tubb4a^{D249N/D249N} mouse model by crossing it with the Tubb4a knock out (KO) mice, which are viable and appear normal. The resulting Tubb4a^{D249N}/KO mice display improved motor function, increased and extended survival (FIG. 20B) and improved motor function (FIG. 20C).

### Example IV

### Overexpression of WT TUBB4A

The information herein above can be applied to rescue the phenotypes associated with tublin mutations by overexpressing of wild-type turbulin.

α and β-tubulins dimerize and cross-dimerize with different tubulin isoforms. While tubulin mutations can cause developmental brain defects, overexpression of wild-type (WT) tubulin out-competes mutant β-tubulin and rescues the associated phenotypes in Drosophila and C. elegans.

In a preferred embodiment we show that over-expressing WT TUBB4A increases expression of myelin genes in an OL-cell line (FIG. 22). Thus, over-expression of WT TUBB4A using an expression vector, (e.g., an Adeno-associated virus (AAV)) will overcome the toxicity of gain-of-function TUBB4A mutations and rescue phenotype in the Tubb4a^{D249N/D249N} mouse. AAV delivery is only one means to deliver a nucleic acid of interest to a cell. Several additional approaches and reagents necessary for delivery are described above.

WT tubulin overexpression can rescue mutant tubulin models. Hence, altering the stoichiometry of tubulins, by increasing WT versus mutated TUBB4A in affected cells, would arrest the neurodegeneration in H-ABC. In a preferred embodiment, proprietary capsids will effectively target our cells of interest, in primate models. In an alternative embodiment, novel viral vectors will target SN, CGC and/or OL to overexpress WT TUBB4A and rescue the phenotype in vitro. Targeting these vectors would improve therapeutic approaches targeted to involved cell types (OL, striatal neurons and cerebellar granule neurons).

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A transgenic mouse having a genome comprising: a promoter, effective to drive expression in a mouse cell, operably linked to a nucleic acid encoding a human, mutant Tubb4-a protein, wherein the human mutated Tubb4-protein is expressed in neurons and/or oligodendrocytes of the mouse and produces a leukodystrophy phenotype, including one or more symptoms selected from motor dysfunction, abnormal gait, ataxia, and decreased survival.

2. The transgenic mouse of claim 1, wherein said Tubb4-A protein is encoded by a nucleic acid listed in Table 1.

3. The transgenic mouse according to claim 1, wherein Tubb4-A protein is a Tubb4a^{D249N} variant.

4. (canceled)

5. A method of identifying a candidate compound for the treatment of leukodystrophy, the method comprising:
contacting the transgenic mouse of claim 2 or a cell, tissue, or organ from neurons of the transgenic mouse of claim 2, with a test compound;
measuring levels of a physical parameter associated with leukodystrophy in the animal, cell, tissue, or organ in the presence and absence of the test compound; and identifying a test compound that alters one or more of these parameters as a candidate compound, said parameters including one or more of a reduction in oligodendrocyte number, hypomyelination, cerebellar granular neuronal loss, striatal neuron loss, dysmyelination, myelination delay, abnormal gait, ataxia, and reduced neuronal survival.

6. (canceled)

7. A method of identifying a candidate therapeutic compound for the treatment of hypomyelination and atrophy of basal ganglia (H-ABC), the method comprising: exposing the transgenic mouse of claim 2, which is a mouse model of H-ABC, to a test compound; measuring one or more parameters of leukodystrophy in the mouse in the presence and absence of the test compound; and identifying a test compound that improves the one or more parameters as a candidate therapeutic compound, wherein said parameters are selected from one or more of reduction in oligodendrocyte number, hypomyelination, cerebellar granular neuronal loss, striatal neuron loss, dysmyelination, myelination delay, abnormal gait, ataxia, and reduced neuronal survival.

8. (canceled)

9. A method of identifying a candidate therapeutic compound for the treatment of hypomyelination and atrophy of basal ganglia (H-ABC), the method comprising:
a) obtaining PBMCs from
i) a subject harboring a TUBB-4A mutation and
ii) from control subjects which lack any TUBB4-A mutation;
b) reprogramming monocytes from step a) to generate induced pluripotent stem cells (iPSCs);
c) directing differentiation of iPSCs towards a striatal spiny neuron fate, said cells expressing one or more markers selected from DARPP32, CTIP2, GABA and FoxP1;
d) contacting said cells with said compound and assessing whether said compound alters a parameter associated with a leukodystrophy phenotype relative to control cells which lack said mutation, wherein said parameter is selected from one or more of reduced cell survival, altered spiny neuron marker expression, and altered cellular morphology or signaling.

10. The method of claim 9, wherein cells are differentiated by applying a dual SMAD inhibition protocol and express DARPP32, CTIP2, GABA and FoxP1 upon differentiation.

11. (canceled)

12. (canceled)

13. The method of claim 9, wherein said TUBB-4A mutation is listed in Table 1.

14. (canceled)

15. (canceled)

16. (canceled)

17. A method for the treatment or prevention of hypomyelination and atrophy of basal ganglia (H-ABC) leukodystrophy comprising administration of an effective amount a compound identified by the method of claim 5, which down modulates expression of both wild-type and mutated TUBB4-A, thereby ameliorating symptoms of H-ABC, wherein said compound is selected from short hairpin RNA (shRNA), short interfering RNA (siRNA), antisense RNA, antisense DNA, chimeric Antisense DNA/RNA, microRNA, and ribozymes that are sufficiently complementary to either a gene or an mRNA encoding TUBB4A.

18. (canceled)

19. The method of claim 17, wherein said compound is selected from siRNA, antisense RNA or an antisense nucleic acid.

20. (canceled)

21. The method of claim 19, wherein said antisense nucleic acid is selected from SEQ ID NO: 1 and SEQ ID NO: 2.

22. A pharmaceutical composition comprising an antisense nucleic acid which down modulates TUBB4A gene expression in a pharmaceutically acceptable carrier, said antisense nucleic acid being selected from SEQ ID NO: 1 or SEQ ID NO:2 for use in the method of claim 21.

23. (canceled)

24. (canceled)

25. The pharmaceutical composition of claim 22, wherein said antisense oligonucleotide is complexed to a nanoparticle or is present in a liposome.

26. (canceled)

27. A pharmaceutical composition comprising a nucleic acid encoding TUBB4A wild type protein for increasing expression of TUBB4A in a cell of interest, in a pharmaceutically acceptable carrier, said nucleic acid optionally being codon optimized.

28. (canceled)

29. The pharmaceutical composition of claim 27, wherein said nucleic acid encodes the amino acid sequence provided in UniProt, accession no. P04350-TBB4A_human, or a nucleic acid encoding a functional fragment thereof wherein said nucleic acid present in an expression or viral vector, said expression or viral vector being complexed to nanoparticle or present in a liposome.

30. (canceled)

31. (canceled)

32. (canceled)

33. A method for genome editing of a TUBB4A encoding nucleic acid, the method comprising:
(a) administering to a subject a vector, wherein the subject is a human, the vector comprising nucleic acid components of a CRISPR-mediated base editor 3 (BE3) system and a guide RNA (gRNA), the gRNA targeting a mutation in a TUBB4A gene; and
(b) introducing a modified codon in the therapeutic gene by base editing the therapeutic gene, wherein the base editing is performed by the vector wherein the modified codon is introduced at a mutation listed in Table 1.

34. The method of claim 33, wherein the base editing occurs prior to disease onset, wherein the disease is a phenotype resulting from the mutation in the TUBB4A gene.

35. (canceled)

36. A method for the treatment or prevention of hypomyelination and atrophy of basal ganglia (H-ABC) leukodystrophy in a subject harboring a mutated TUBB4A gene, comprising administration of an effective amount the composition of claim 29 which increases expression of wild type TUBB4-A protein, thereby ameliorating symptoms of H-ABC.

37. (canceled)

38. An siRNA for use in the method of claim 17 which downmodulates expression of a nucleic acid expressing Tubb4a.

39. (canceled)

40. A kit for practicing the method of claim 9.
